(19) United States
(12) Patent Application Publication
(10) Pub. No.: US 2002/0081299 A1
(43) Pub. Date: Jun. 27, 2002

(54) Hair cell disorders

(76) Inventor: Wei-Qiang Gao, Foster City, CA (US)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/849,868

(22) Filed: May 4, 2001

Related U.S. Application Data
(60) Provisional application No. 60/107,522, filed on Nov. 7, 1998.

(51) Int. Cl. A61K 39/395; A61K 38/17

(52) U.S. Cl. 424/145.1; 424/93.7; 514/12

(57) ABSTRACT

Ligands which bind to the HER2 and/or HER3 receptors are useful as inner-ear-supporting cell growth factors to enhance proliferation-mediated generation of new hair cells.

### BACKGROUND OF THE INVENTION

1. Field of the Invention

This application relates to inducing, promoting, or enhancing the growth, proliferation, repair, generation, or regeneration of inner ear tissue, particularly inner ear epithelial hair cells and supporting cells. More particularly, this application relates to potently stimulating supporting cell proliferation and enhancing proliferation-mediated generation of new hair cells. In addition, this application provides methods, compositions and devices for prophylactic and therapeutic treatment of inner ear disorders and conditions, particularly sensorineural hearing and balance impairments. This invention relates to the use of HER2 ligands, in particular heregulin polypeptides, as inner-ear-supporting cell growth factors.

2. Description of Background

Hearing and balance impairments are serious handicaps which affect millions of people. Hearing impairments can be attributed to a wide variety of causes, including infections, mechanical injury, loud sounds, aging, and chemical-induced ototoxicity that damages neurons and/or hair cells of the peripheral auditory system. The peripheral auditory system consists of auditory receptors, hair cells in the organ of Corti, and primary auditory neurons, the spiral ganglion neurons in the cochlea. Spiral ganglion neurons (“SGN”) are primary afferent auditory neurons that deliver signals from the peripheral auditory receptors, the hair cells in the organ of Corti, to the brain through the cochlear nerve. The eighth nerve connects the primary auditory neurons in the spiral ganglia to the brain stem. The eight nerve also connects vestibular ganglion neurons (“VGN”), which are primary afferent sensory neurons responsible for balance and which deliver signals from the utricle, saccule and ampullae of the inner ear to the brain, to the brainstem. Destruction of primary afferent neurons in the spiral ganglia and hair cells has been attributed as a major cause of hearing impairments. Damage to the peripheral auditory system is responsible for a majority of hearing deficits (Dublin, 1976; Rybak, 1986; Lim, 1986; Pryor, 1994).

Hearing loss or impairment is a common occurrence for mammals. Impairment anywhere along the auditory pathway from the external auditory canal to the central nervous system may result in hearing loss or balance impairment. Auditory apparatus can be divided into the external and middle ear, inner ear and auditory nerve and central auditory pathways. While having some variations from species to species, the general characterization is common for all mammals. Auditory stimuli are mechanically transmitted through the external auditory canal, tympanic membrane, and ossicular chain to the inner ear. The middle ear and mastoid process are normally filled with air. Disorders of the external and middle ear usually produce a conductive hearing loss by interfering with this mechanical transmission. Common causes of a conductive hearing loss include obstruction of the external auditory canal, as can be caused by aural atresia or cerumen; thickening or perforation of the tympanic membrane, as can be caused by trauma or infection; fixation or resorption of the components of the ossicular chain; and obstruction of the Eustachian tube, resulting in a fluid-filled middle-ear space. Auditory information is transduced from a mechanical signal to a neurally conducted electrical impulse by the action of neuro-epithelial cells (hair cells) and SGN in the inner ear. All central fibers of SGN form synapses in the cochlear nucleus of the pontine brain stem. The auditory projections from the cochlear nucleus are bilateral, with major nuclei located in the inferior colliculus, medial geniculate body of the thalamus, and auditory cortex of the temporal lobe. The number of neurons involved in hearing increases dramatically from the cochlea to the auditory brain stem and the auditory cortex. All auditory information is transduced by a limited number of hair cells, which are the sensory receptors of the inner ear, of which the so-called inner hair cells, numbering a comparative few, are critically important, since they form synapses with approximately 90 percent of the primary auditory neurons. By comparison, at the level of the cochlear nucleus, the number of neural elements involved is measured in the hundreds of thousands. Thus, damage to a relatively few cells in the auditory periphery can lead to substantial hearing loss or balance impairment. Hence, many causes of sensorineural loss can be ascribed to lesions in the inner ear. This hearing loss and balance impairment can be progressive. In addition, the hearing becomes significantly less acute because of changes in the anatomy of the ear as the animal ages.

During embryogenesis, the vestibular ganglion, spiral ganglion, and the otic vesicle are derived from the same neurogenic ectoderm, the otic placode. The vestibular and auditory systems thus share many characteristics including peripheral neuronal innervations of hair cells and central projections to the brainstem nuclei. Both of these systems are sensitive to ototoxins that include therapeutic drugs, antineoplastic agents, contaminants in foods or medicines, and environmental and industrial pollutants. Ototoxic drugs include the widely used chemotherapeutic agent cisplatin and its analogs (Fleischman et al., 1975; Stadnicki et al., 1975; Nakai et al., 1982; Berggren et al., 1990; Dublin, 1976; Hood and Berlin, 1986), commonly used aminoglycoside antibiotics, e.g. gentamicin, for the treatment of infections caused by Gram-negative bacteria, (Sera et al., 1987; Hinojosa and Lerner, 1987; Bareggi et al., 1990), quinine and its analogs, salicylate and its analogs, and loop-diuretics.

The toxic effects of these drugs on auditory cells and spiral ganglion neurons are often the limiting factor for their therapeutic usefulness. For example, antibacterial aminoglycosides such as gentamicins, streptomycins, kanamycins, tobramycins, and the like are known to have serious toxicity, particularly ototoxicity and nephrotoxicity, which reduce the usefulness of such antimicrobial agents (see Goodman and Gilman's The Pharmacological Basis of Therapeutics, 6th ed., A. Goodman Gilman et al., eds; Macmillan Publishing Co., Inc., New York, pp. 1169-71 (1980) or most recent edition). Aminoglycoside antibiotics are generally utilized as broad spectrum antimicrobials effective against, for example, gram-positive, gram-negative and acid-fast bacteria. Susceptible microorganisms include Escherichia spp., Hemophilus spp., Listeria spp., Pseudomonas spp., Nocardia spp., Yersinia spp., Klebsiella spp., Enterobacter spp., Salmonella spp., Staphylococcus spp., Streptococcus spp., Mycobacteria spp., Shigella spp., and Serratia spp. Nonetheless, the aminoglycosides are used primarily to treat infections caused by gram-negative bacteria and, for instance, in combination with penicillins for the synergistic effects. As implied by the generic name for the family, all the aminoglycoside antibiotics contain aminosugars in glycosidic linkage. Otitis media is a term used to describe infections of the middle ear, which infections are very common, particularly in children. Typically antibiotics are systemically administered for infections of the middle ear, e.g., in a responsive or prophylactic manner. Systemic administration of antibiotics to combat middle ear infection generally results in a prolonged lag time to achieve therapeutic levels in the middle ear, and requires high initial doses in order to achieve such levels. These drawbacks complicate the ability to obtain therapeutic levels and may preclude the use of some antibiotics altogether. Systemic administration is most often effective when the infection has reached advanced stages, but at this point permanent damage may already have been done to the middle and inner ear structure. Clearly, ototoxicity is a dose-limiting side-effect of antibiotic administration. For example, nearly 75% of patients given 2 grams of streptomycin daily for 60 to 120 days displayed some vestibular impairment, whereas at 1 gram per day, the incidence decreased to 25% (U.S. Pat. No. 5,059,591). Auditory impairment was observed: from 4 to 15% of patients receiving 1 gram per day for greater than 1 week develop measurable hearing loss, which slowly becomes worse and can lead to complete permanent deafness if treatment continues.

Ototoxicity is also a serious dose-limiting side-effect for cisplatin, a platinum coordination complex, that has proven effective on a variety of human cancers including testicular, ovarian, bladder, and head and neck cancer. Cisplatin damages auditory and vestibular systems (Fleischman et al., 1975; Stadnicki et al., 1975; Nakai et al., 1982; Carenza et al., 1986; Sera et al., 1987; Bareggi et al., 1990). Salicylates, such as aspirin, are the most commonly used therapeutic drugs for their anti-inflammatory, analgesic, anti-pyretic and anti-thrombotic effects. Unfortunately, they have ototoxic side effects. They often lead to tinnitus (“ringing in the ears”) and temporary hearing loss (Myers and Bernstein, 1965). However, if the drug is used at high doses for a prolonged time, the hearing impairment can become persistent and irreversible, as reported clinically (Jarvis, 1966).

Accordingly, there exists a need for means to prevent, reduce or treat the incidence and/or severity of inner ear disorders and hearing impairments involving inner ear tissue, particularly inner ear hair cells, and optionally, the associated auditory nerves. Of particular interest are those conditions arising as an unwanted side-effect of ototoxic therapeutic drugs including cisplatin and its analogs, aminoglycoside antibiotics, salicylate and its analogs, or loop diuretics. In addition, there exits a need for methods which will allow higher and thus more effective dosing with these ototoxicity-inducing pharmaceutical drugs, while concomitantly preventing or reducing ototoxic effects caused by these drugs. What is needed is a method that provides a safe, effective, and prolonged means for prophylactic or curative treatment of hearing impairments related to inner ear tissue damage, loss, or degeneration, particularly ototoxin-induced, and particularly involving inner ear hair cells. The present invention provides compositions and methods to achieve these goals and others as well.

A need continues to exist for a method of treatment hearing and balance impairments and other conditions associated with damage to hair cells or their supporting cells.

### SUMMARY OF THE INVENTION

In general an object of the invention is to provide a method of inducing, promoting, or enhancing the growth, proliferation, repair, or regeneration of inner ear tissue, particularly inner ear hair cells and their supporting cells for the purpose of promoting repair and healing of inner tissue damage or injury.

Accordingly, one object of this invention is to provide a method of treating inner ear disorders and conditions in patients, primarily human patients, in need of such treatment. A further object is to provide a method of inducing inner-ear-supporting cell growth, generation, and development, which leads to generation of new hair cells.

In one aspect of this invention, it has now been discovered that these objects and the broader objective of treating conditions associated with hair cell or inner-ear-supporting cell damage and injury are achieved by administering to a patient in need of such treatment an effective amount of a heregulin ligand, preferably a polypeptide or fragment thereof. These heregulin polypeptides, include HRG-α, HRG-β1, HRG-β2, HRG-β3 and other heregulin polypeptides which cross-react with antibodies directed against these family members and/or which are substantially homologous as defined below and includes heregulin variants such as N-terminal and C-terminal fragments thereof. A preferred heregulin is the ligand disclosed in FIG. 1A-1D and further designated HRG-α. Other preferred heregulins are the ligands disclosed in FIG. 2A-2E, and designated HRG-β1; disclosed in FIG. 3A-3E designated HRG-β2; and disclosed in FIG. 4A-4C designated HRG-β3.

In another aspect, the invention provides a method in which heregulin agonist antibodies are administered to achieve the objects of the invention. In this embodiment, HER2/HER3 or fragments thereof (which also may be synthesized by in vitro methods) are fused (by recombinant expression or an in vitro peptidyl bond) to an immunogenic polypeptide and this fusion polypeptide, in turn, is used to raise antibodies against a HER2/HER3 epitope. Agonist antibodies are recovered from the serum of immunized animals. Alternatively, monoclonal antibodies are prepared from in vito cells or in vivo immunized animals in conventional fashion. If desired, the agonist antibodies may be obtained by phage display selection from a phage library of antibodies or antibody fragments. Preferred antibodies identified by routine screening will bind to the receptor, but will not substantially cross-react with any other known ligands such as EGF, and will activate the HER receptors HER2 or HER3, preferably Her2. In addition, antibodies may be selected that are capable of binding specifically to individual family members of heregulin family, e.g. HRG-α, HRG-β1, HRG-β2, HRG-β3, and which are agonists thereof.

In general, the invention is a method of regenerating and/or repairing hair cell or inner-ear-supporting cell injury by stimulating growth and proliferation of inner-ear-supporting cells to enhance generation of new hair cells. The hair cells may be injured by many types of insults, for example, injury due to surgical incision or resection, chemical or smoke inhalation or aspiration, chemical or biochemical ulceration, cell damage due to viral or bacterial infection, etc The inner-ear-supporting cells which may be affected by the method of the invention include any inner-ear-supporting cell which expresses HER2 or HER3, preferably Her3. The method of the invention stimulates growth and proliferation of the inner-ear-supporting cells leading to generation of new hair cells to repair and re-establish the sensorineural contacts in the inner ear to allow the affected tissues to develop normal physiological functions more quickly.

Accordingly, one embodiment of the invention is a method of inducing inner-ear-supporting cell growth by contacting a inner-ear-supporting cell which expresses HER2 receptor with an effective amount of a HER2 activating ligand.

A further embodiment is a method of treating inner ear hair cell injury, caused by ototoxins or acoustic assault for example, by administering to a patient in need thereof an effective amount of a HER2 activating ligand.

### BRIEF DESCRIPTION OF THE DRAWINGS

FIG. 1A-1D show the deduced amino acid sequence (SEQ ID NO:1) for the cDNA sequence (SEQ ID NO:2) contained in a clone obtained according to U.S. Pat. No. 5,367,060. The initiating methionine (Met) of HRG-α is at position 45.

FIG. 2A-2E show the deduced amino acid sequence (SEQ ID NO:3) and cDNA sequence (SEQ ID NO:4) of a potential coding sequence of a clone obtained according to U.S. Pat. No. 5,367,060 for HRG-β1. The initiating Met is at M31.

FIG. 3A-3E show the deduced amino acid sequence (SEQ ID NO:5) and cDNA sequence (SEQ ID NO:6) of a nucleotide sequence of a clone obtained according to U.S. Pat. No. 5,367,060 for HRG-β2.

FIG. 4A-4C show the deduced amino acid sequence (SEQ ID NO:7) and cDNA sequence (SEQ ID NO:8) of a nucleotide sequence of a clone obtained according to U.S. Pat. No. 5,367,060 for HRG-β3.

FIG. 5A-5D show the deduced amino acid sequence (SEQ ID NO:9) and cDNA sequence (SEQ ID NO:10) of a nucleotide sequence of a clone obtained according to U.S. Pat. No. 5,367,060 for HRG-β2-like protein.

FIG. 6A-6C show a comparison of the amino acid homologies of several known heregulins α, β1, β2, β2-like and β3 in descending order and illustrates the amino acid insertions, deletions, and substitutions that characterize these forms of heregulin (SEQ ID NOS: 1, 3, 5, 9, and 7).

FIG. 7A-7C show the deduced amino acid sequence (SEQ ID NO:11) and cDNA sequence (SEQ ID NO:12) of -HRG. The hydrophobic region is underlined. The EGF-like domain is shaded, cysteine residues in the EGF-like domain are circled. N-linked glycosylation sites are marked above the nucleic acid sequence with a ( ).

FIG. 8 shows the cDNA sequence (SEQ ID NO:13) and amino acid sequence (SEQ ID NO:14) of SMDF. An EGF-like domain and the apolar and uncharged domains (i.e. “apolar I” consisting of residues from about 48-62 and “apolar II” consisting of residues from about 76-100) are underlined. Cysteines in the EGF-like domain and in the “cysteine knot” in the unique N-terminal domain (“NTD-cys knot”) are boxed. The stop codon is denoted by the letter “O”.

FIG. 9 shows the effect of various polypeptide growth factors on the proliferaition of cells in the rat utricular sheet hair cell layer, as indicated by the number of BrdU positive cells per sheet compared to control.

FIG. 10 shows the dose-dependent proliferation effect of heregulin on cells in the rat utricular sheet hair cell layer, as indicated by the number of BrdU positive cells per sheet compared to control.

FIGS. 11A-D show autoradiography of tritiated-thymidine labeled cells in supporting cell and hair cell layer in gentamicin-treated utricles in response to heregulin treatment. FIGS. A-D are views from similarly treated organotypic rat utricular whole mounts.

FIG. 12 shows the cell count of tritiated-thymidine labeled cells in supporting cell and hair cell layer in gentamicin-treated utricles (shown in FIGS. 11A-D) in response to heregulin treatment compared to control.

FIG. 13 shows the RNA concentration of heregulin and the recepters Her2, Her3 and Her4 in RAN isolated from the inner ear sensory epithelium layer.

FIG. 14 shows localization of Her2, a heregulin receptor, in the inner ear sensory epithelium, as indicated by immunostaining the P0 cochlea and adult utricle with labeled monoclonal antibody to Her2.

### DETAILED DESCRIPTION OF THE PREFERRED EMBODIMENTS

Heregulin ligands, in particular polypeptides and agonist antibodies thereof, have affinity for and stimulate the HER2, and less preferably HER3, receptors or combinations thereof in autophosphorylation. Included within the definition of heregulin ligands, in addition to HRG-α, HRG-β1, HRG-β2, HRG-β3 and HRG-β2-like, are other polypeptides binding to the HER2 receptor, which bear substantial amino acid sequence homology to HRG-α or HRG-β1. Such additional polypeptides fall within the definition of heregulin as a family of polypeptide ligands that bind to the HER2 receptors.

Heregulin polypeptides bind with varying affinities to the HER2 receptors. It is also known that heterodimerization of HER2 with HER3 occurs with subsequent receptor cross-phosphorylation as described above. In the present invention, inner-ear-supporting cell growth and/or proliferation is induced when a heregulin protein interacts and binds with an individual receptor molecule or a receptor dimer such that receptor phosphorylation is induced. Binding and activation of HER2 or Her3 or combinations thereof, therefore, is meant to include activation of any form of the receptor necessary for receptor activation and biologic function including monomeric receptor and dimeric receptor forms. Dimeric receptor forms may be referred to below, for example, as HER2/HER3.

The HER (ErbB) family belongs to the subclass I receptor tyrosine kinase superfamily and consists of three distinct receptors, HER2, HER3, and HER4. A ligand for this ErbB family is the protein heregulin (HRG), a multidomain containing protein with at least 15 distinct isoforms.

Transduction of signals that regulate cell growth and differentiation is regulated in part by phosphorylation of various cellular proteins. Protein tyrosine kinases are enzymes that catalyze this process. Receptor protein tyrosine kinases are believed to direct cellular grovth via ligand-stimulated tyrosine phosphorylation of intracellular substrates. Growth factor receptor protein tyrosine kinases of the class I subfamily include the 170 kDa epidermal growth factor receptor (EGFR) encoded by the erbB1 gene. erbB1 has been causally implicated in human malignancy. In particular, increased expression of this gene has been observed in more aggressive carcinomas of the breast, bladder, lung and stomach.

The second member of the class I subfamily, p185^{neu}, was originally identified as the product of the transforming gene from neuroblastomas of chemically treated rats. The neu gene (also called erbB2 and HER2) encodes a 185 kDa receptor protein tyrosine kinase. Amplification and/or overexpression of the human HER2 gene correlates with a poor prognosis in breast and ovarian cancers (Slamon et al., Science 235:177-182 (1987); and Slamon et al., Science 244:707-712 (1989)). Overexpression of HER2 has been correlated with other carcinomas including carcinomas of the stomach, endometrium, salivary gland, lung, kidney, colon and bladder. Accordingly, Slamon et al. in U.S. Pat. No. 4,968,603 describe and claim various diagnostic assays for determining HER2 gene amplification or expression in tumor cells. Slamon et al. discovered that the presence of multiple gene copies of HER2 oncogene in tumor cells indicates that the disease is more likely to spread beyond the primary tumor site, and that the disease may therefore require more aggressive treatment than might otherwise be indicated by other diagnostic factors. Slamon et al. conclude that the HER2 gene amplification test, together with the determination of lymph node status, provides greatly improved prognostic utility.

A further related gene, called erbB3 or HER3, has also been described. See U.S. Pat. No. 5,183,884; Kraus et al., Proc. Natl. Acad Sci. USA 86:9193-9197 (1989); EP Pat Appin No 444,961A1; and Kraus et al., Proc. Natl. Acad. Sci. USA 90:2900-2904 (1993). Kraus et al. (1989) discovered that markedly elevated levels of erbB3 mRNA were present in certain human mammary tumor cell lines indicating that erbB3, like erbB1 and erbB2, may play a role in human malignancies. Also, Kraus et al. (1993) showed that EGF-dependent activation of the ErbB3 catalytic domain of a chimeric EGFR/ErbB3 receptor resulted in a proliferative response in transfected NIH-3T3 cells. This is now believed to be the result of endogenous ErbB1 or ErbB2 in NIH-3T3. Furthermore, these researchers demonstrated that some human mammary tumor cell lines display a significant elevation of steady-state ErbB3 tyrosine phosphorylation further indicating that this receptor may play a role in human malignancies. The role of erbB3 in cancer has been explored by others. It has been found to be overexpressed in breast (Lemoine et al., Br. J. Cancer 66:1116-1121 (1992)), gastrointestinal (Poller et al., J. Pathol. 168:275-280 (1992), Rajkumer et al., J. Pathol. 170:271-278 (1993), and Sanidas et al., Int. J. Cancer 54:935-940 (1993)), and pancreatic cancers (Lemoine et al., J. Pathol. 168:269-273 (1992), and Friess et al., Clinical Cancer Research 1:1413-1420 (1995)).

The class I subfamily of growth factor receptor protein tyrosine kinases has been further extended to include the HER4/Erb4 receptor. See EP Pat Appin No 599,274; Plowman et al., Proc. Nati. Acad. Sci. USA 90:1746-1750 (1993); and Plowman et al., Nature 366:473-475 (1993). Plowman et al. found that increased HER4 expression closely correlated with certain carcinomas of epithelial origin, including breast adenocarcinomas. Diagnostic methods for detection of human neoplastic conditions (especially breast cancers) which evaluate HER4 expression are described in EP Pat Appin No. 599,274.

The quest for the activator of the HER2 oncogene has lead to the discovery of a family of heregulin polypeptides. These proteins appear to result from alternate splicing of a single gene which was mapped to the short arm of human chromosome 8 by Orr-Urtreger et al., Proc. Natf. Acad. Sci. USA 90:1867-1871(1993). See also Lee and Wood, Genomics, 16:790-791 (1993).

Holmes et al. isolated and cioned a family of polypeptide activators for the HER2 receptor which they called heregulin-α (HRG-α), heregulin-β1 (HRG-β1), heregulin-β2 (HRG-β2), heregulin-β2-like (HRG-β2-like), and heregulin-β3 (HRG-β3). See Holmes et al., Science 256:1205-1210 (1992); WO 92/20798; and U.S. Pat. No. 5,367,060. The 45 kDa polypeptide, HRG-α, was purified from the conditioned medium of the MDA-MB-231 human breast cancer cell line. These researchers demonstrated the ability of the purified heregulin polypeptides to activate tyrosine phosphorylation of the HER2 receptor in MCF7 breast tumor cells. Furthermore, the mitogenic activity of the heregulin polypeptides on SK-BR-3 cells (which express high levels of the HER2 receptor) was illustrated. Like other growth factors which belong to the EGF family, soluble HRG polypeptides appear to be derived from a membrane bound precursor (called pro-HRG) which is proteolytically processed to release the 45 kDa soluble form. These pro-HRGs lack a N-terminal signal peptide.

While heregulins are substantially identical in the first 213 amino acid residues, they are classified into two major types, α and β, based on two variant EGF-like domains which differ in their C-terminal portions. Nevertheless, these EGF-like domains are identical in the spacing of six cysteine residues contained therein. Based on an amino acid sequence comparison, Holmes et al. found that between the first and sixth cysteines in the EGF-like domain, HRGs were 45% similar to heparin-binding EGF-like growth factor (HB-EGF), 35% identical to amphiregulin (AR), 32% identical to TGF-α, and 27% identical to EGF. The 44 kDa neu differentiation factor (NDF), which is the rat equivalent of human HRG, was first described by Peles et al., Cell, 69:205-216 (1992); and Wen et al., Cell, 69:559-572 (1992). Like the HRG polypeptides, NDF has an immunoglobulin (Ig) homology domain followed by an EGF-like domain and lacks a N-terminal signal peptide. Subsequently, Wen et al., Mol. Cell. Biol., 14(3):1909-1919 (1994) carried out “exhaustive cloning” to extend the family of NDFs. This work revealed six distinct fibroblastic pro-NDFs. Adopting the nomenclature of Holmes et al., the NDFs are classified as either α or β polypeptides based on the sequences of the EGF-like domains. Isoforms 1 to 4 are characterized on the basis of the variable just a membrane stretch (between the EGF-like domain and transmembrane domain). Also, isoforms a, b and c are described which have variable length cytoplasmic domains. These researchers conclude that different NDF isoforms are generated by alternative splicing and perform distinct tissue-specific functions. See also EP 505 148; WO 93/22424; and WO 94/28133 concerning NDF.

Falls et al., Cell, 72:801-815 (1993) describe another member of the heregulin family which they call acetylcholine receptor inducing activity (ARIA) polypeptide. The chicken-derived ARIA polypeptide stimulates synthesis of muscle acetylcholine receptors. See also WO 94/08007. ARIA is a β-type heregulin and lacks the entire spacer region rich in glycosylation sites between the Ig-like domain and EGF-like domain of HRGα, and HRGβ1-β3.

Marchionni et al., Nature, 362:312-318 (1993) identified several bovine-derived proteins which they call glial growth factors (GGFs). These GGFs share the Ig-like domain and EGF-like domain with the other heregulin proteins described above, but also have an amino-terminal kringle domain. GGFs generally do not have the complete glycosylated spacer region between the Ig-like domain and EGF-like domain. Only one of the GGFs, GGFII, possessed a N-terminal signal peptide. See also WO 92118627; WO 94/00140; WO 94/04560; WO 94/26298; and WO 95/32724 which refer to GGFs and uses thereof.

Ho et al. in J. Biol. Chem. 270(4):14523-14532 (1995) describe another member of the heregulin family called sensory and motor neuron-derived factor (SMDF). This protein has an EGF-like domain characteristic of all other heregulin polypeptides but a distinct N-terminal domain. The major structural difference between SMDF and the other heregulin polypeptides is the lack in SMDF of the Ig-like domain and the “glyco” spacer characteristic of all the other heregulin polypeptides. Another feature of SMDF is the presence of two stretches of hydrophobic amino acids near the N-terminus.

While the heregulin polypeptides were first identified based on their ability to activate the HER2 receptor (see Holmes et al., supra), it was discovered that certain ovarian cells expressing neu and neu-transfected fibroblasts did not bind or crosslink to NDF, nor did they respond to NDF to undergo tyrosine phosphorylation (Peles et al., EMBO J. 12:961-971 (1993)). This indicated another cellular component was necessary for conferring full heregulin responsiveness. Carraway et al. subsequently demonstrated that ¹²⁵I-rHRGβ1₁₇₇₋₂₄₄ bound to NIH-3T3 fibroblasts stably transfected with bovine erbB3 but not to non-transfected parental cells. Accordingly, they conclude that ErbB3 is a receptor for HRG and mediates phosphorylation of intrinsic tyrosine residues as well as phosphorylation of ErbB2 receptor in cells which express both receptors. Carraway et al., J. Biol. Chem. 269(19):14303-14306 (1994). Sliwkowski et al., J. Biol. Chem. 269(20):14661-14665 (1994) found that cells transfected with HER3 alone show low affinities for heregulin, whereas cells transfected with both HER2 and HER3 show higher affinities.

This observation correlates with the “receptor cross-talking” described previously by Kokai et al., Cell 58:287-292 (1989); Stern et al., EMBO J. 7:995-1001 (1988) and King et al., 4:13-18 (1989). These researchers found that binding of EGF to the EGFR resulted in activation of the EGFR kinase domain and cross-phosphorylation of p185^{HER2}. This is believed to be a result of ligand-induced receptor heterodimerization and the concomitant cross-phosphorylation of the receptors within the heterodimer (Wada et al., Cell 61:1339-1347 (1990)).

Plowman and his colleagues have similarly studied p185^{HER4}/p185^{HER2} activation. They expressed p185^{HER2} alone, p185^{HER4} alone, or the two receptors together in human T lymphocytes and demonstrated that heregulin is capable of stimulating tyrosine phosphorylation of p185^{HER4}, but could only stimulate p185^{HER2} phosphorylation in cells expressing both receptors. Plowman et al., Nature 336:473-475 (1993).

The biological role of heregulin has been investigated by several groups. For example, Falls et al., (discussed above) found that ARIA plays a role in myotube differentiation, namely affecting the synthesis and concentration of neurotransmitter receptors in the postsynaptic muscle cells of motor neurons. Corfas and Fischbach demonstrated that ARIA also increases the number of sodium channels in chick muscle. Corfas and Fischbach, J. Neuroscience, 13(5): 2118-2125 (1993). It has also been shown that GGFII is mitogenic for subconfluent quiescent human myoblasts and that differentiation of clonal human myoblasts in the continuous presence of GGFII results in greater numbers of myotubes after six days of differentiation (Sklar et al., J. Cell Biochem., Abst. W462, 18D, 540 (1994)). See also WO 94/26298 published Nov. 24, 1994.

Holmes et al., supra, found that HRG exerted a mitogenic effect on mammary cell lines (such as SK-BR-3 and MCF-7). The mitogenic activity of GGFs on Schwann cells has also been reported. See, e.g., Brockes et al., J. Biol. Chem. 255(18):8374-8377 (1980); Lemke and Brockes, J. Neurosci. 4:75-83 (1984); Brockes et al., J. Neuroscience 4(1):75-83 (1984); Brockes et al., Ann. Neurol. 20(3):317-322 (1986); Brockes, J., Methods in Enzym., 147: 217-225 (1987) and Marchionni et al., supra. Schwann cells constitute important glial cells which provide myelin sheathing around the axons of neurons, thereby forming individual nerve fibers. Thus, it is apparent that Schwann cells play an important role in the development, function and regeneration of peripheral nerves. The implications of this from a therapeutic standpoint have been addressed by Levi et al., J. Neuroscience 14(3):1309-1319 (1994). Levi et al. discuss the potential for construction of a cellular prosthesis comprising human Schwann cells which could be transplanted into areas of damaged spinal cord. Methods for culturing Schwann cells ex vivo have been described. See WO 94/00140 and Li et al., J. Neuroscience 16(6):2012-2019 (1996).

Pinkas-Kramarski et al. found that NDF seems to be expressed in neurons and glial cells in embryonic and adult rat brain and primary cultures of rat brain cells, and suggested that it may act as a survival and maturation factor for astrocytes (Pinkas-Kramarski et al., PNAS, USA 91:9387-9391 (1994)). Meyer and Birchmeier, PNAS, USA 91:1064-1068 (1994) analyzed expression of heregulin during mouse embryogenesis and in the perinatal animal using in situ hybridization and RNase protection experiments. See also Meyer et al., Development 124(18):3575-3586 (1997). These authors conclude that, based on expression of this molecule, heregulin plays a role in vivo as a mesenchymal and neuronal factor. Similarly, Danilenko et al., Abstract 3101, FASEB 8(4-5):A535 (1994); Danilenko et al., Journal of Clinical Investigation 95(2): 842-851 (1995), found that the interaction of NDF and the HER2 receptor is important in directing epidermal migration and differentiation during wound repair.

Ram et al., Journal of Cellular Physiology 163:589-596 (1995) evaluated the mitogenic activity of NDF on the immortalized human mammary epiphelial cell line MCF-10A. Danilenko et al., J. Clin. Invest 95:842-851 (1995) investigated whether NDF would influence epidermal migration in an in vivo model of excisional deep partial-thickness wound repair. It is reported that there were no statistically significant differences in proliferating basal and superbasal keratinocytes in control wounds vs. wounds treated with rhNDF-α₂. Marikovsky et al., Oncogene 10:1403-1411 (1995), studied the proliferative responses of an aneuploid BALB/MK continuous keratinocyte cell line and evaluated the effects of α- and β-isoforms of NDF on epidermal keratinocytes.

The relationship between the structure and function of new proteins can be investigated using any of a variety of available mutational analysis techniques. Examples of such techniques include alanine scanning mutagenesis and phagemid display. Alanine scanning can be used to identify active residues (i.e., residues that have a significant effect on protein function) in a protein or protein domain. For example, Cunningham and Wells used alanine scanning to identify residues in human growth hormone that were important for binding its receptor. Cunningham and Wells, Science 244:1081-1085 (1989). In alanine scanning, a gene encoding the protein or domain to be scanned is inserted into an expression vector, and mutagenesis is carried out to generate a series of vectors that encode proteins or domains in which sequential residues are converted to alanine. The encoded proteins or domain are expressed from these vectors, and the activities of the alanine-substituted variants are then tested to identify those with altered activity. An alteration in activity indicates that the residue at the alanine-substituted position is an active residue.

Phagemid display was developed to allow the screening of a large number of variant polypeptides for a particular binding activity. Smith and Parmley demonstrated that foreign peptides can be “displayed” efficiently on the surface of filamentous phage by inserting short gene fragments into gene III of the fd phage. Smith, Science 228:1315-1317 (1985); Parmley and Smith, Gene 73:305-318 (1985). The gene III coat protein is present in about five copies at one end of the phage particle. The modified phage were termed “fusion phage” because they displayed the foreign peptides fused to the gene III coat protein. As each fusion phage particle displayed approximately five copies of the fusion protein, this mode of phage display was termed “polyvalent display.”

Scott et al. and Cwirla et al. showed that fusion phage libraries could be screened by sequential affinity selections known as “panning.” Scott et al., Science 249:386-390 (1990); Cwirla et al., PNAS USA 87:6378-6382 (1990). However, early efforts to select high affinity fusion phage failed, presumably due to the polyvalence of the phage particles. This problem was solved with the development of a “monovalent” phage display system in which the fusion protein is expressed at a low level from a phagemid and a helper phage provides a large excess of wild-type coat protein. Bass et al., Proteins 8:309-314 (1990); Lowman et al., Biochem. 30:10832-10838 (1991). Monovalent phage display can be used to generate and screen a large number of variant polypeptides to isolate those that bind with high affinity to a target of interest.

I. Definitions

In general, the following words or phrases have the indicated definition when used in the description, examples, and claims.

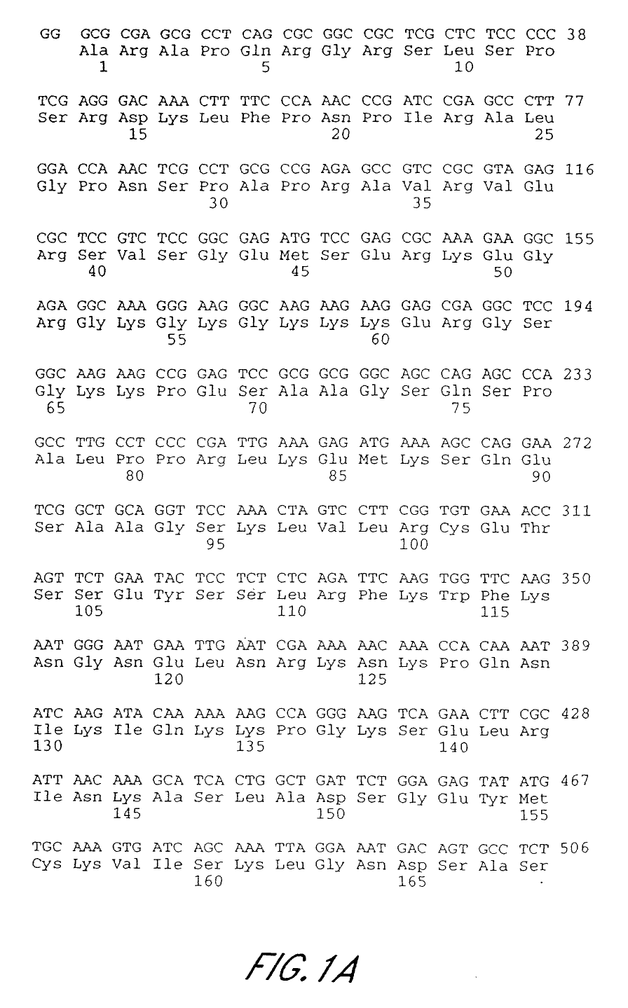

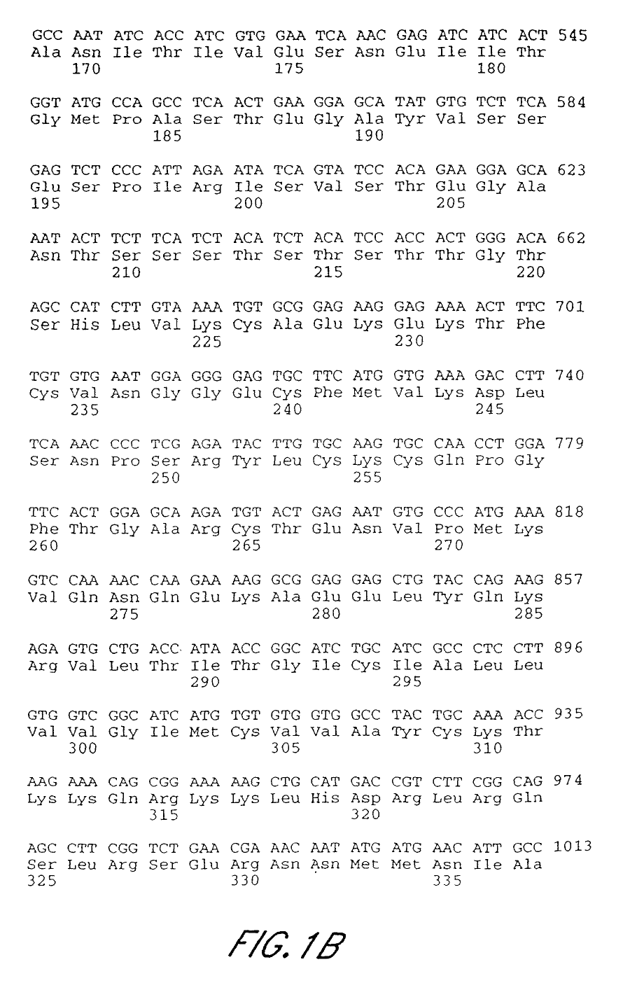

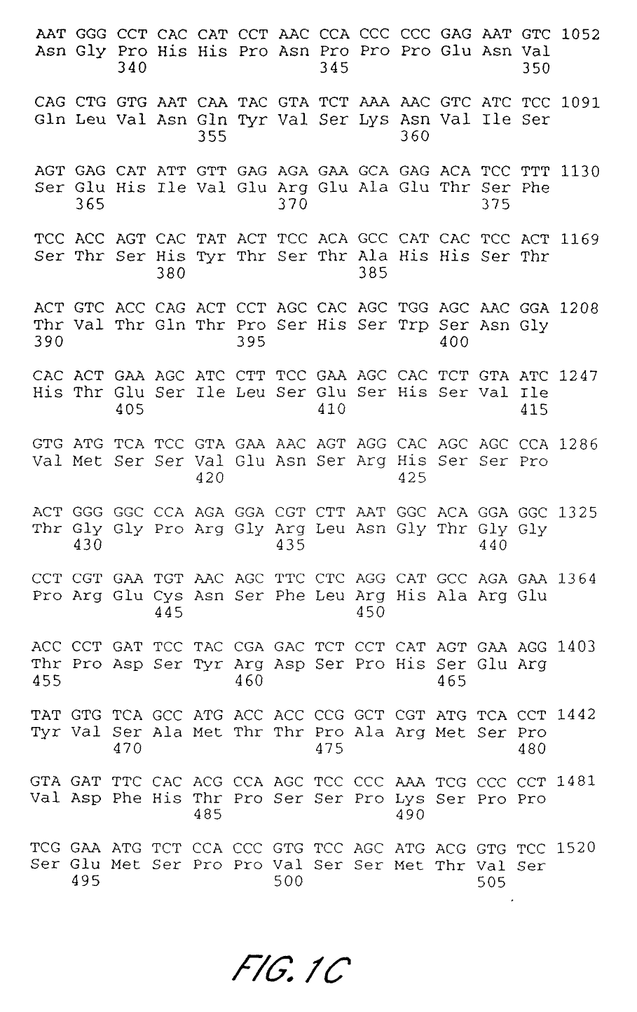

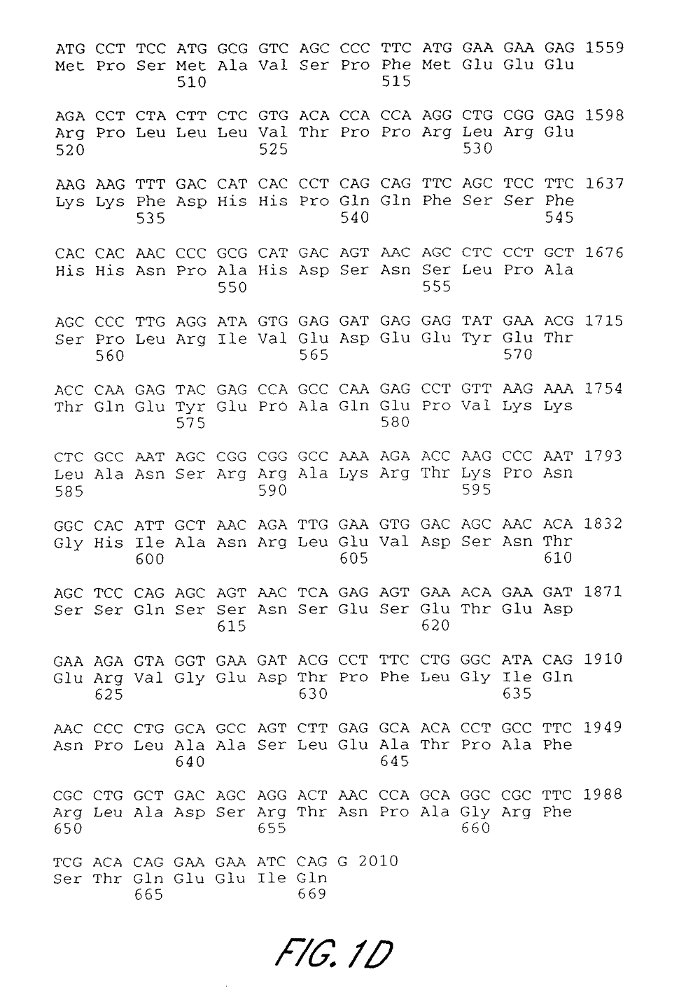

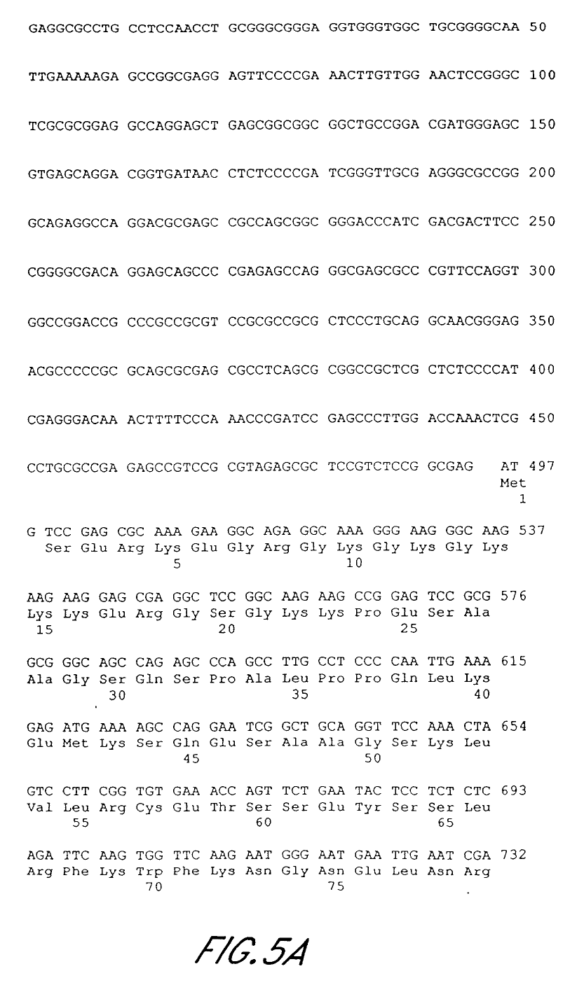

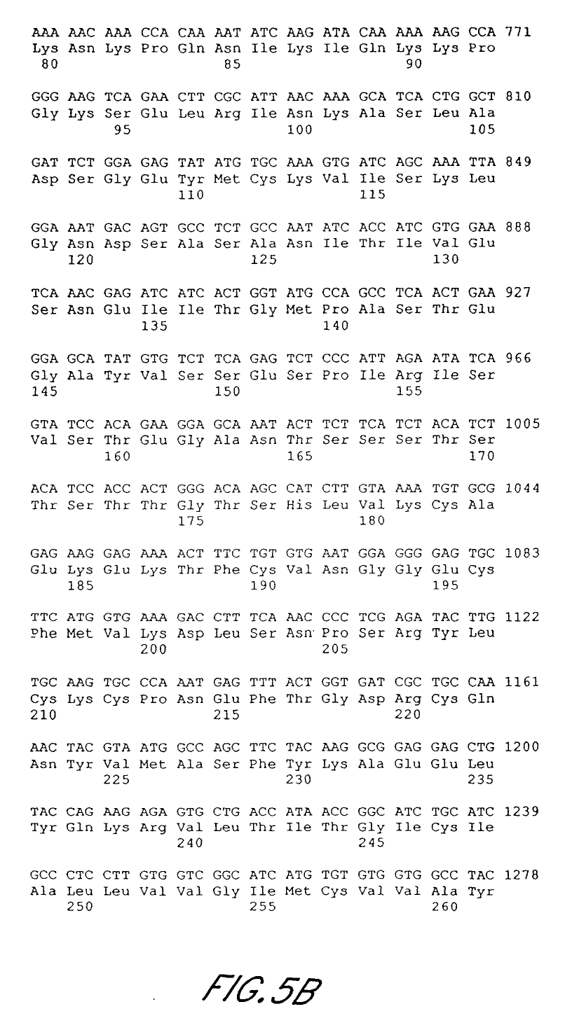

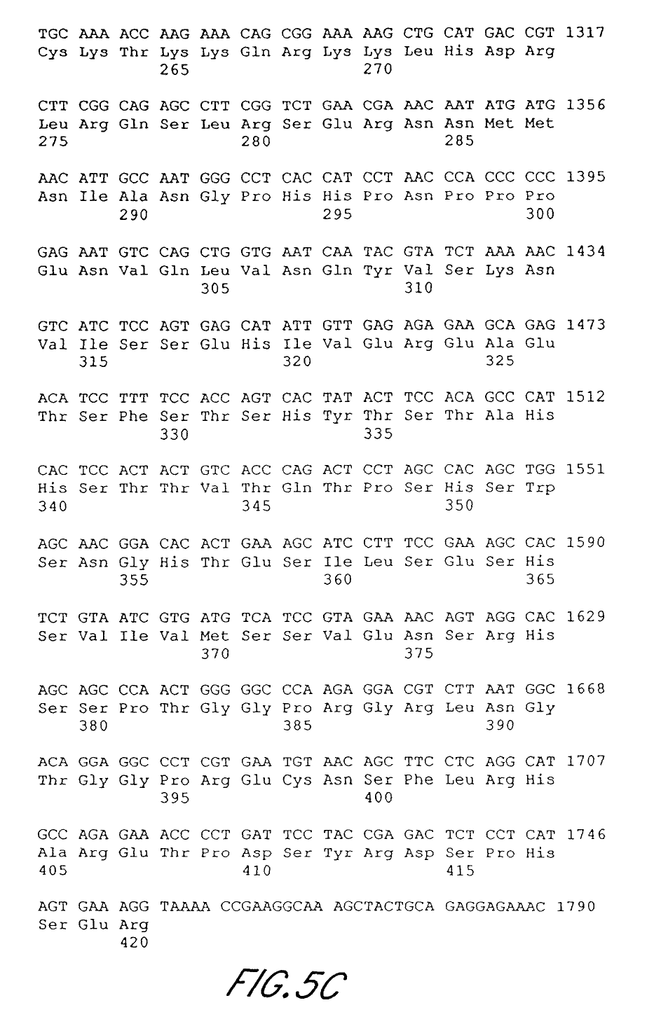

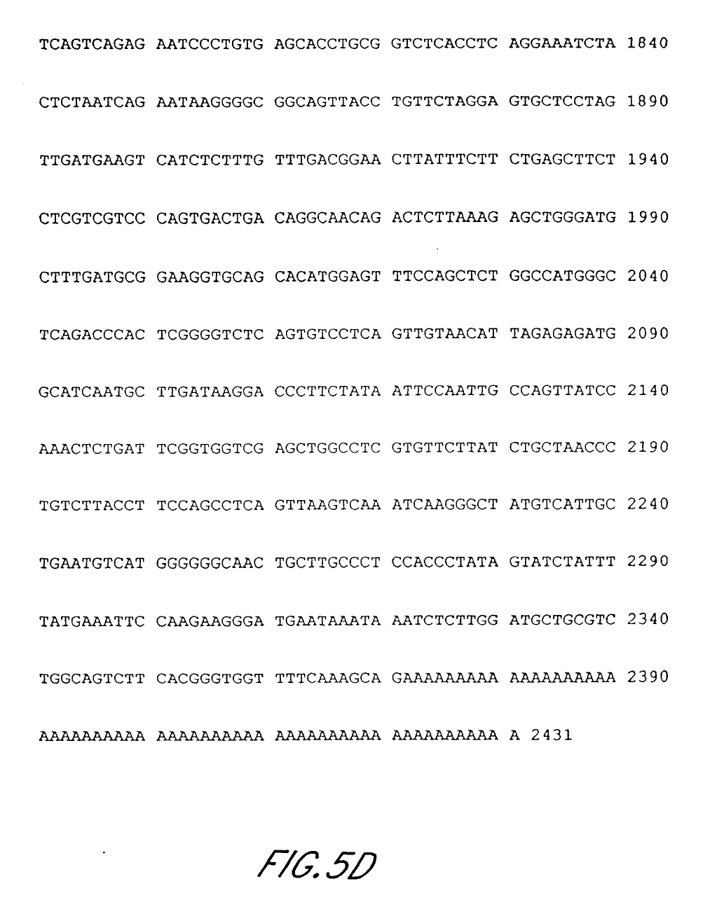

“Heregulin” ligand is defined herein to be any isolated ligand, preferably a polypeptide sequence which possesses a biological property of a naturally occurring heregulin polypeptide that binds and activates Her2. Ligands within the scope of this invention include the heregulin polypeptides discussed in detail herein. Heregulin includes the polypeptides shown in FIGS. 1A-1D, 2A-2E, 3A-3E, 4A-4C, 5A-5D, 6A-6C, and 7A-7C and mammalian analogues thereof. Variants can be prepared by the methods described below, optionally together with alanine scanning and phage display techniques known in the art. Cunningham and Wells, Science 244:1081-85 (1989); Bass et al., Proteins 8:309-14 (1990); Lowman et al., Biochem. 30:10832-38 (1991).

The term a “normal” hair cell or inner-ear-supporting cell means an hair cell or inner-ear-supporting cell which is not transformed, i.e., is non-cancerous and/or non-immortalized. Further, the normal hair cell or inner-ear-supporting cell is preferably not aneuploid. Aneuploidy exists when the nucleus of a cell does not contain an exact multiple of the haploid number of chromosomes, one or more chromosomes being present in greater or lesser number than the rest. Typical properties of transformed cells which fall outside the scope of this invention include the ability to form tumors when implanted into immune-deprived mice (nude mice), the ability to grow in suspension or in semi-solid media such as agar, a loss of contact inhibition allowing piling up of cells into colonies or foci, a loss of dependence on growth factors or serum, cell death if cells are inhibited from growing, and disorganization of actin filaments. Specifically included within the invention are normal cells which will not form tumors in mice, grow attached to plastic or glass (are anchorage dependent), exhibit contact inhibition, require serum-containing hormones and growth factors, remain viable if growth is arrested by lack of serum, and contain well-organized actin filaments. Although the normal inner-ear-supporting cells are preferably not cultured cells, also suitable for the invention are non-transformed, non-immortalized epithelial cells isolated from mammalian tissue. These isolated cells may be cultured for several generations (up to about 10 or even 50 generations) in the presence of a heregulin in order to induce growth and/or proliferation of the isolated inner ear supporting cell sample, that is, to expand the sample. The expanded sample can then be reintroduced into the mammal for the purpose of repopulating the hair cell or inner-ear-supporting cell tissue (re-epithelialization). This is particularly useful for repairing tissue injury or damage.

“Biological property” for the purposes herein means an in vivo biologic or antigenic function or activity that is directly or indirectly performed by an heregulin sequence (whether in its native or denatured conformation), or by any subsequence thereof. Biologic functions include receptor binding, any enzyme activity or enzyme modulatory activity, any carrier binding activity, any hormonal activity, any activity in promoting or inhibiting adhesion of cells to extracellular matrix or cell surface molecules, or any structural role. However, biologic functions do not include antigenic functions, i.e. possession of an epitope or antigenic site that is capable of cross-reacting with antibodies raised against a naturally occurring heregulin polypeptide.

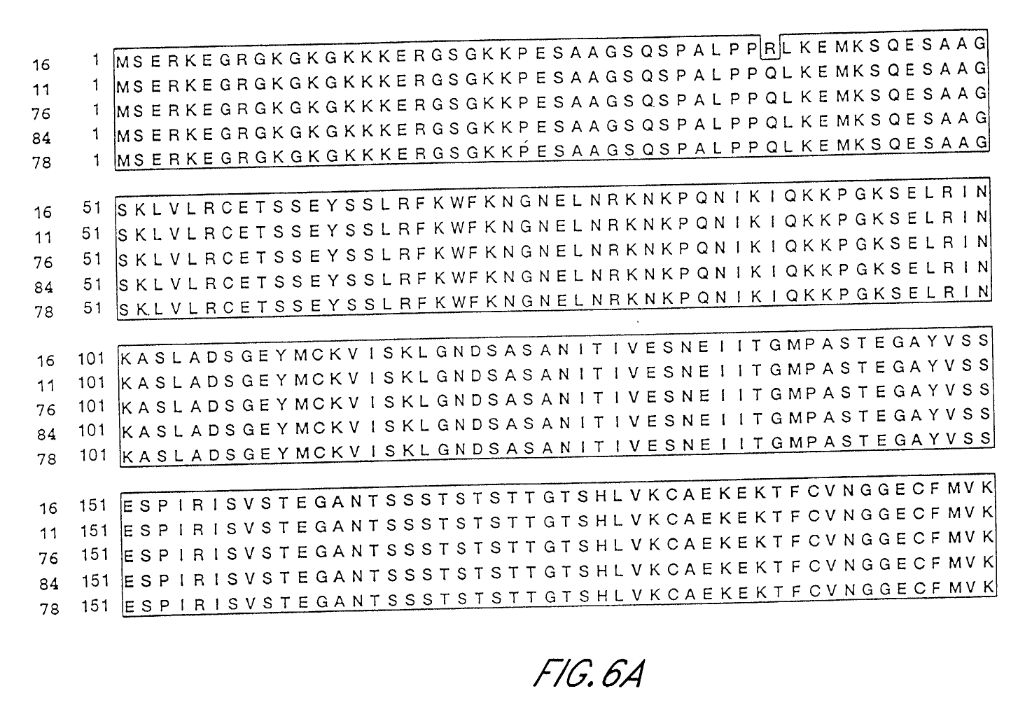

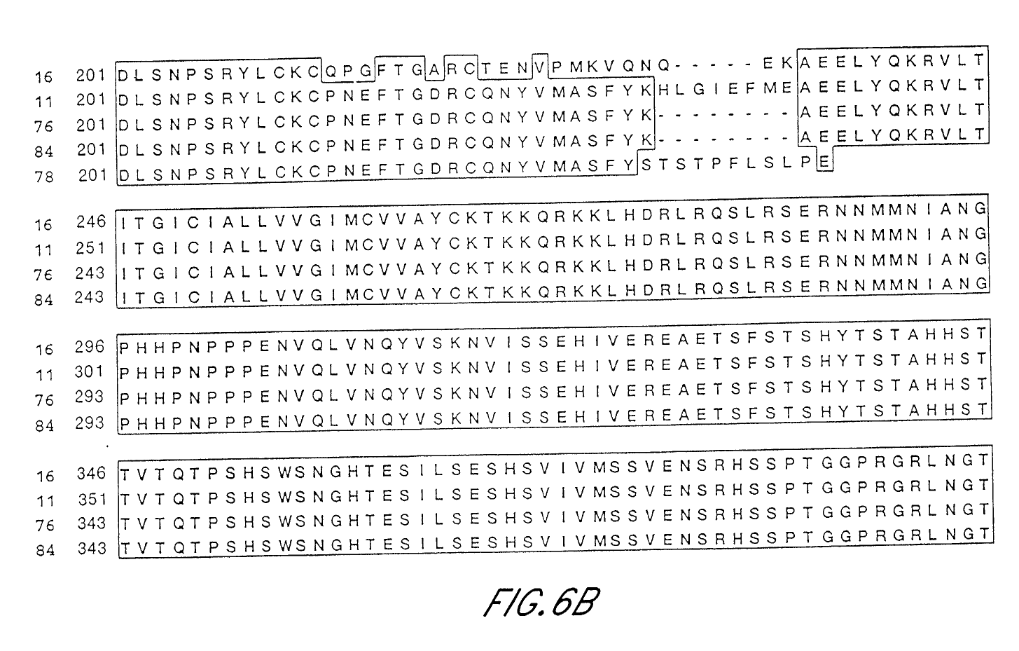

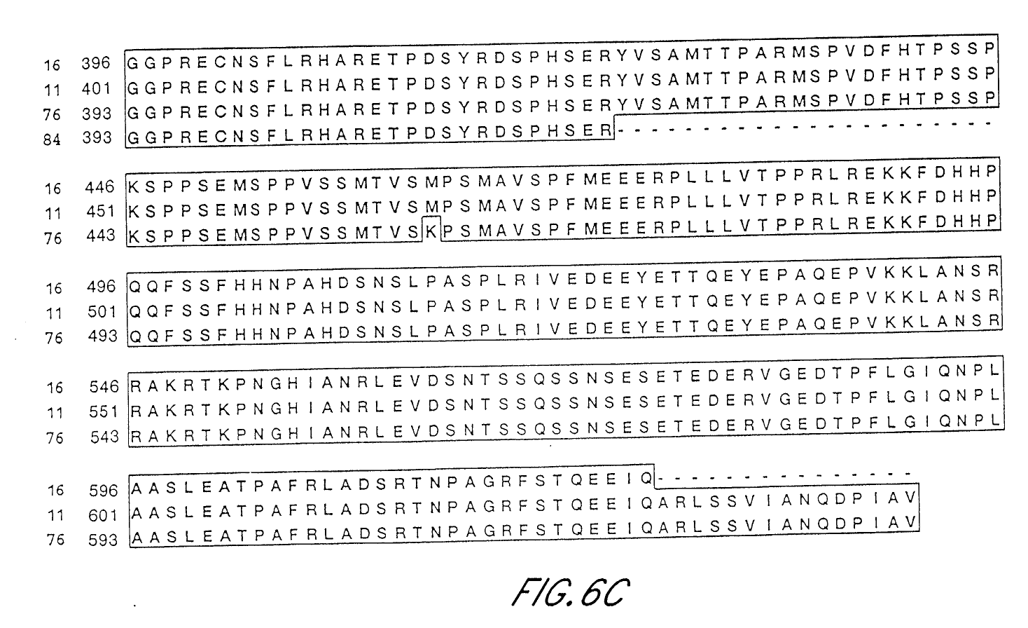

“Biologically active” heregulin is defined herein as a polypeptide sharing a biologic function of an heregulin sequence which may (but need not) in addition possess an antigenic function. A principal known effect or function of heregulin is as a ligand polypeptide having a qualitative biological activity of binding to HER2 resulting in the activation of the receptor tyrosine kinase (an “activating ligand”). One test for activating ligands is the heregulin tyrosine autophosphorylation assay described below. Included within the scope of heregulin as that term is used herein are heregulin having translated mature amino acid sequences of the complete human heregulin as set forth herein; deglycosylated or unglycosylated derivatives of heregulin, amino acid sequence variants of heregulin sequence, and derivatives of heregulin, which are capable of exhibiting a biological property in common with heregulin. While native heregulin is a membrane-bound polypeptide, soluble forms, such as those forms lacking a functional transmembrane domain, are also included within this definition. In particular, included are polypeptide fragments of heregulin sequence which have an N-terminus at any residue from about S216 to about A227, and its C-terminus at any residue from about K268 to about R286, and the homologous sequences shown in FIG. 6A-C, hereinafter referred to collectively for all heregulins as the growth factor domain (GFD).

“Antigenically active” heregulin is defined as a polypeptide that possesses an antigenic function of an heregulin and which may (but need not) in addition possess a biologic function.

In preferred embodiments, antigenically active heregulin is a polypeptide that binds with an affinity of at least about 10⁻⁹ I/mole to an antibody raised against a naturally occurring heregulin sequence. Ordinarily the polypeptide binds with an affinity of at least about 10⁻⁸ I/mole. Most preferably, the antigenically active heregulin is a polypeptide that binds to an antibody raised against one of heregulins in its native conformation. Heregulin in its native conformation generally is heregulin as found in nature which has not been denatured by chaotropic agents, heat or other treatment that substantially modifies the three dimensional structure of heregulin as determined, for example, by migration on nonreducing, nondenaturing sizing gels. Antibody used in this determination may be rabbit polyclonal antibody raised by formulating native heregulin from a non-rabbit species in Freund's complete adjuvant, subcutaneously injecting the formulation, and boosting the immune response by intraperitoneal injection of the formulation until the titer of anti-heregulin antibody plateaus.

Ordinarily, biologically or antigenically active heregulin will have an amino acid sequence having at least 75% amino acid sequence identity with a given heregulin sequence, more preferably at least 80%, even more preferably at least 90%, and most preferably at least 95%. Identity or homology with respect to an heregulin sequence is defined herein as the percentage of amino acid residues in the candidate sequence that are identical with heregulin residues in the heregulin of FIG. 6A-6C, after aligning the sequences and introducing gaps, if necessary, to achieve the maximum percent homology, and not considering any conservative substitutions as part of the sequence identity. None of N-terminal, C-terminal or internal extensions, deletions, or insertions into heregulin sequence shall be construed as affecting homology.

Thus, the biologically active and antigenically active heregulin polypeptides that are the subject of this invention include each entire heregulin sequence; fragments thereof having a consecutive sequence of at least 5, 10, 15, 20, 25, 30 or 40 amino acid residues from heregulin sequence; amino acid sequence variants of heregulin sequence wherein an amino acid residue has been inserted N- or C-terminal to, or within, heregulin sequence or its fragment as defined above; amino acid sequence variants of heregulin sequence or its fragment as defined above has been substituted by another residue. heregulin polypeptides include those containing predetermined mutations by, e.g., site-directed or PCR mutagenesis, and other animal species of heregulin polypeptides such as rabbit, rat, porcine, non-human primate, equine, murine, and ovine heregulin and alleles or other naturally occurring variants of the foregoing and human sequences; derivatives of heregulin or its fragments as defined above wherein heregulin or its fragments have been covalently modified by substitution, chemical, enzymatic, or other appropriate means with a moiety other than a naturally occurring amino acid (for example a detectable moiety such as an enzyme or radioisotope); glycosylation variants of heregulin (insertion of a glycosylation site or deletion of any glycosylation site by deletion, insertion or substitution of appropriate amino acid); and soluble forms of heregulin, such as HRG-GFD or those that lack a functional transmembrane domain.

“Isolated” means a ligand, such as heregulin, which has been identified and separated and/or recovered from a component of its natural environment. Contaminant components of its natural environment are materials which would interfere with diagnostic or therapeutic uses for heregulin, and may include proteins, hormones, and other substances. In preferred embodiments, heregulin will be purified (1) to greater than 95% by weight of protein as determined by the Lowry method or other validated protein determination method, and most preferably more than 99% by weight, (2) to a degree sufficient to obtain at least 15 residues of N-terminal or internal amino acid sequence by use of the best commercially available amino acid sequenator marketed on the filing date hereof, or (3) to homogeneity by SDS-PAGE using Coomassie blue or, preferably, silver stain. Isolated heregulin includes heregulin in situ within recombinant cells since at least one component of heregulin natural environment will not be present. Isolated heregulin includes heregulin from one species in a recombinant cell culture of another species since heregulin in such circumstances will be devoid of source polypeptides. Ordinarily, however, isolated heregulin will be prepared by at least one purification step.

In accordance with this invention, heregulin nucleic acid is RNA or DNA containing greater than ten bases that encodes a biologically or antigenically active heregulin, is complementary to nucleic acid sequence encoding such heregulin, or hybridizes to nucleic acid sequence encoding such heregulin and remains stably bound to it under stringent conditions.

Preferably, heregulin nucleic acid encodes a polypeptide sharing at least 75% sequence identity, more preferably at least 80%, still more preferably at least 85%, even more preferably at 90%, and most preferably 95%, with an heregulin sequence. Preferably, the heregulin nucleic acid that hybridizes contains at least 20, more preferably at least about 40, and most preferably at least about 90 bases.

Isolated heregulin nucleic acid includes a nucleic acid that is identified and separated from at least one containment nucleic acid with which it is ordinarily associated in the natural source of heregulin nucleic acid. Isolated heregulin nucleic acid thus is present in other than in the form or setting in which it is found in nature. However, isolated heregulin encoding nucleic acid includes heregulin nucleic acid in ordinarily heregulin-expressing cells where the nucleic acid is in a chromosomal location different from that of natural cells or is otherwise flanked by a different DNA sequence than that found in nature. Nucleic acid encoding heregulin may be used in specific hybridization assays, particularly those portions of heregulin encoding sequence that do not hybridize with other known DNA sequences. “Stringent conditions” are those that (1) employ low ionic strength and high temperature for washing, for example, 0.015 M NACl/0.0015 M sodium citrate/0/1% NaDodSO₄ at 50° C.; (2) employ during hybridization a denaturing agent such as formamide, for example, 50% (vol/vol) formamide with 0.1% bovine serum albumin, 0.1% Ficoll, 0.1% polyvinylpyrrolidone, 50 mM sodium phosphate buffer at pH 6.5 with 750 mM NaCI, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 g/ml), 0.1% SDS, and 10% dextran sulfate at 42° C., with washes at 42° C. in 0.2×SSC and 0.1% SDS.

The term “control sequences” refers to DNA sequences necessary for the expression of an operably linked coding sequence in a particular host organism. The control sequences that are suitable for prokaryotes, for example, include a promoter, optionally an operator sequence, a ribosome binding site, and possibly, other as yet poorly understood sequences. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a secretory leader, contiguous and in reading phase. However enhancers do not have to be contiguous. Linking is accomplished by ligation at convenient restriction sites. If such sites do not exist, then synthetic oligonucleotide adaptors or linkers are used in accord with conventional practice.

An “exogenous” element is defined herein to mean nucleic acid sequence that is foreign to the cell, or homologous to the cell but in a position within the host cell nucleic acid in which the element is ordinarily not found.

As used herein, the expressions “cell”, “cell line”, and “cell culture” are used interchangeably, and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. It will be clear from the context where distinct designations are intended.

“Plasmids” are designated by a lower case “p” preceded and/or followed by capital letters and/or numbers. The starting plasmids herein are commercially available, are publicly available on an unrestricted basis, or can be constructed from such available plasmids in accord with published procedures. In addition, other equivalent plasmids are known in the art and will be apparent to the ordinary artisan.

“Restriction enzyme digestion” of DNA refers to catalytic cleavage of the DNA with an enzyme that acts only at certain locations in the DNA. Such enzymes are called restriction endonucleases, and the sites for which each is specific is called a restriction site. The various restriction enzymes used herein are commercially available and their reaction conditions, cofactors, and other requirements as established by the enzyme suppliers are used. Restriction enzymes commonly are designated by abbreviations composed of a capital letter followed by other letters representing the microorganism from which each restriction enzyme originally was obtained, and then a number designating the particular enzyme. In general, about 1 mg of plasmid or DNA fragment is used with about 1-2 units of enzyme in about 20 ml of buffer solution. Appropriate buffers and substrate amounts for particular restriction enzymes are specified by the manufacturer. Incubation of about 1 hour at 37° C. is ordinarily used, but may vary in accordance with the supplier's instructions. After incubation, protein or polypeptide is removed by extraction with phenol and chloroform, and the digested nucleic acid is recovered from the aqueous fraction by precipitation with ethanol. Digestion with a restriction enzyme may be followed with bacterial alkaline phosphatase hydrolysis of the terminal 5′ phosphates to prevent the two restriction cleaved ends of a DNA fragment from “circularizing” or forming a closed loop that would impede insertion of another DNA fragment at the restriction site. Unless otherwise stated, digestion of plasmids is not followed by 5′ terminal dephosphorylation. Procedures and reagents for dephosphorylation are conventional as described in sections 1.56-1.61 of Sambrook et al., (Molecular Cloning: A Laboratory Manual New York: Cold Spring Harbor Laboratory Press, 1989).

“Recovery” or “isolation” of a given fragment of DNA from a restriction digest means separation of the digest on polyacrylamide or agarose gel by electrophoresis, identification of the fragment of interest by comparison of its mobility versus that of marker DNA fragments of known molecular weight, removal of the gel section containing the desired fragment, and separation of the gel from DNA. This procedure is known generally. For example, see Lawn et al., Nucleic Acids Res. 9:6103-6114 (1981), and Goeddel et al., Nucleic Adds Res. 8:4057 1980).

“Northern analysis” is a method used to identify RNA sequences that hybridize to a known probe such as an oligonucleotide, DNA fragment, cDNA or fragment thereof, or RNA fragment. The probe is labeled with a radioisotope such as ³²P, or by biotinylation, or with an enzyme. The RNA to be analyzed is usually electrophoretically separated on an agarose or polyacrylamide gel, transferred to nitrocellulose, nylon, or other suitable membrane, and hybridized with the probe, using standard techniques well known in the art such as those described in sections 7.39-7.52 of Sambrook et al., supra.

“Ligation” refers to the process of forming phosphodiester bonds between two nucleic acid fragments. To ligate the DNA fragments together, the ends of the DNA fragments must be compatible with each other. In some cases, the ends will be directly compatible after endonuclease digestion. However, it may be necessary to first convert the staggered ends commonly produced after endonuclease digestion to blunt ends to make them compatible for ligation. To blunt the ends, the DNA is treated in a suitable buffer for at least 15 minutes at 15° C. with about 10 units of the Klenow fragment of DNA polymerase I or T4 DNA polymerase in the presence of the four deoxyribonucleotide triphosphates. The DNA is then purified by phenol-chloroform extraction and ethanol precipitation. The DNA fragments that are to be ligated together are put in solution in about equimolar amounts. The solution will also contain ATP, ligase buffer, and a ligase such as T4 DNA ligase at about 10 units per 0.5 mg of DNA. If the DNA is to be ligated into a vector, the vector is first linearized by digestion with the appropriate restriction endonuclease(s). The linearized fragment is then treated with bacterial alkaline phosphatase, or calf intestinal phosphatase to prevent self-ligation during the ligation step.

“Preparation” of DNA from cells means isolating the plasmid DNA from a culture of the host cells. Commonly used methods for DNA preparation are the large and small-scale plasmid preparations described in sections 1.25-1.33 of Sambrook et al., supra. After preparation of the DNA, it can be purified by methods well known in the art such as that described in section 1.40 of Sambrook et al., supra.

“Oligonucleotides” are short-length, single- or double-stranded polydeoxynucleotides that are chemically synthesized by known methods (such as phosphotriester, phosphite, or phosphoramidite chemistry, using solid phase techniques such as described in EP 266,032, published May 4, 1988, or via deoxynucleoside H-phosphonate intermediates as described by Froehler et al., Nucl. Acids Res. 14:5399-5407, 1986. They are then purified on polyacrylamide gels.

The technique of “polymerase chain reaction,” or “PCR,” as used herein generally refers to a procedure wherein minute amounts of a specific piece of nucleic acid, RNA and/or DNA, are amplified as described in U.S. Pat. No. 4,683,195, issued Jul. 28, 1987. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al., Cold Spring Harbor Symp. Quant. Biol. 51: 263 (1987); Erlich, ed., PCR Technology, (Stockton Press, NY, 1989). As used herein, PCR is considered to be one, but not the only, example of a nucleic acid polymerase reaction method for amplifying a nucleic acid test sample, comprising the use of a known nucleic acid (DNA or RNA) as a primer, and utilizes a nucleic acid polymerase to amplify or generate a specific piece of nucleic acid or to amplify or generate a specific piece of nucleic acid which is complementary to a particular nucleic acid.

The “heregulin tyrosine autophosphorylation assay” to detect the presence or bioactivity of heregulin ligands can be used to monitor the purification of a ligand for the HER2 receptors. This assay is based on the assumption that a specific ligand for the receptor will stimulate autophosphorylation of the receptor, in analogy with EGF and its stimulation of EGF receptor autophosphorylation. See Sadich et al., Anal. Biochem. 235:207-214 (1996). MDA-MB453 cells or MCF7 cells which contain high levels of p185^{HER2} receptors but negligible levels of human EGF receptors, were obtained from the American Type Culture Collection, Rockville, Md. (ATCC No HTB-131) and maintained in tissue culture with 10% fetal calf serum in DMEM/Hams F12 (1:1) media. For assay, the cells were trypsinized and plated at 150,000 cells/well in 24 well dishes (Costar). After incubation with serum containing media overnight, the cells were placed in serum free media for 2-18 hours before assay. Test samples of 100 uL aliquots were added to each well. The cells were incubated for 5-30 minutes (typically 30 min) at 37° C. and the media removed. The cells in each well were treated with 100 uL SDS gel denaturing buffer (SEPROSOL, Enpotech, Inc.) and the plates heated at 100° C. for 5 minutes to dissolve the cells and denature the proteins. Aliquots from each well were electrophoresed on 5-20% gradient SDS gels (NOVEX, Encinitas, Calif.) according to the manufacturer's directions. After the dye front reached the bottom of the gel, the electrophoresis was terminated and a sheet of PVDF membrane (PROBLOTT, ABI) was placed on the gel and the proteins transferred from the gel to the membrane in a blotting chamber (BioRad) at 200 mAmps for 30-60 min. After blotting, the membranes were incubated with TRIS buffered saline containing 0.1% TWEEN 20 detergent buffer with 5% BSA for 2-18 hrs to block nonspecific binding, and then treated with a mouse anti-phosphotyrosine antibody (Upstate Biological Inc., N.Y.). Subsequently, the membrane blots were treated with goat anti-mouse antibody conjugated to alkaline phosphatase. The gels were developed using the PROTOBLOT System from Promega. After drying the membranes, the density of the bands corresponding to p185^{HER2} in each sample lane was quantitated with a Hewlett Packard SCANJET Plus Scanner attached to a Macintosh computer. The number of receptors per cell in the MDA-MB453 cells is such that under these experimental conditions the p185^{HER2} receptor protein is the major protein which is labeled.

“Protein microsequencing” was accomplished based upon the following procedures. Proteins from the final HPLC step were either sequenced directly by automated Edman degradation with a model 470A Applied Biosystems gas phase sequencer equipped with a 120A PTH amino acid analyzer or sequenced after digestion with various chemicals or enzymes. PTH amino acids were integrated using the CHROMPERFECT data system (Justice Innovations, Palo Alto, Calif.). Sequence interpretation was performed on a VAX 11/785 Digital Equipment Corporation computer as described (Henzel et al., J. Chromatography 404:41-52 (1987)). In some cases, aliquots of the HPLC fractions were electrophoresed on 5-20% SDS polyacrylamide gels, electrotransferred to a PVDF membrane (PROBLOTT, ABI, Foster City, Calif.) and stained with Coomassie Brilliant Blue (Matsudaira, P., J. Biol. Chem. 262:10035-10038, 1987). The specific protein was excised from the blot for N terminal sequencing. To determine internal protein sequences, HPLC fractions were dried under vacuum (SPEEDVAC), resuspended in appropriate buffers, and digested with cyanogen bromide, the lysine-specific enzyme Lys-C (Wako Chemicals, Richmond, Va.) or Asp-N (Boehringer Mannheim, Indianapolis, Ind.). After digestion, the resultant peptides were sequenced as a mixture or were resolved by HPLC on a C4 column developed with a propanol gradient in 0.1% TFA before sequencing as described above.

“Antibodies” (Abs) and “immunoglobulins” (Igs) are glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lymph system and at increased levels by myelomas.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Fv” is the minimum antibody fragment which contains a complete antigen-recognition and -binding site. This region consists of a dimer of one heavy chain and one light chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Also included are Fab fragments having the addition of a few residues at the carboxyl terminus of the heavy chain CH1 domain including one or more cysteine(s) from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2. The subunit structures and three-dimensional configurations of different classes of immunoglobulins are well known.

The term “antibody” herein is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

“Antibody fragments” comprise a portion of a full length antibody, generally the antigen binding or variable domain thereof. Examples of antibody fragments include Fab, Fab′, F(ab′)₂, and Fv fragments; diabodies; linear antibodies; single-chain antibody molecules; and multispecific antibodies formed from antibody fragments.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature 352:624-628 (1991) and Marks et al., J. Mol. Biol. 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

“Humanized” forms of non-human (e.g., murine) antibodies are chimeric antibodies which contain minimal sequence derived from non-human immunoglobulin. For the most part, humanized antibodies are human immunoglobulins (recipient antibody) in which hypervariable region residues of the recipient are replaced by hypervariable region residues from a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Reichmann et al., Nature 332:323-329 (1988); and Presta, Curr. Op. Struct. Biol. 2:593-596 (1992).

“Single-chain Fv” or “sFv” antibody fragments comprise the V_{H} and V_{L} domains of antibody, wherein these domains are present in a single polypeptide chain. Generally, the Fv polypeptide further comprises a polypeptide linker between the V_{H} and V_{L} domains which enables the sFv to form the desired structure for antigen binding. For a review of sFv see Pluckthun in The Pharmacology of Monoclonal Antibodies, vol. 113, Rosenburg and Moore eds. Springer-Verlag, New York, pp. 269-315 (1994).

The term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA 90:6444-6448 (1993).

The expression “linear antibodies” when used throughout this application refers to the antibodies described in Zapata et al. Protein Eng. 8(10):1057-1062 (1995). Briefly, these antibodies comprise a pair of tandem Fd segments (V_{H}-C_{H}1-V_{H}-C_{H}1) which form a pair of antigen binding regions. Linear antibodies can be bispecific or monospecific.

II. Use and Preparation of Heregulin Sequences

H. Preparation of Heregulin Sequences, Including Variants

The system to be employed in preparing heregulin sequence will depend upon the particular heregulin sequence selected. If the sequence is sufficiently small heregulin may be prepared by in vitro polypeptide synthetic methods. Most commonly, however, heregulin will be prepared in recombinant cell culture using the host-vector systems described below. Suitable heregulin includes any biologically active and antigenetically active heregulin.

In general, mammalian host cells will be employed, and such hosts may or may not contain post-translational systems for processing heregulin preprosequences in the normal fashion. If the host cells contain such systems then it will be possible to recover natural subdomain fragments from the cultures. If not, then the proper processing can be accomplished by transforming the hosts with the required enzyme(s) or by supplying them in an in vitro method. However, it is not necessary to transform cells with the complete prepro or structural genes for a selected heregulin when it is desired to only produce fragments of heregulin sequences. For example, a start codon is ligated to the 5′ end of DNA encoding an HRG-GFD, this DNA is used to transform host cells and the product expressed directly as the Met N-terminal form (if desired, the extraneous Met may be removed in vitro or by endogenous N-terminal demethionylases). Alternatively, HRG-GFD is expressed as a fusion with a signal sequence recognized by the host cell, which will process and secrete the mature HRG-GFD as is further described below. Amino acid sequence variants of native HRG-GFD sequences are produced in the same way.

Heregulin sequences located between the first N-terminal mature residue and the first N-terminal residue of HRG-GFD sequence, termed HRG-NTD, may function at least in part as an unconventional signal sequence or as a normally circulating carrier/precursor for HRG-GFD having unique biological activity. HRG-NTD is produced in the same fashion as the full length molecule but from expression of DNA in which a stop codon is located at the C-terminus of HRG-NTD. In addition, heregulin variants are expressed from DNA encoding protein in which both the GFD and NTD domains are in their proper orientation but which contain an amino acid insertion, deletion or substitution at the GFD-NTD cleavage site (located within the sequence VKC) which inhibits or prevents proteolytic cleavage of the NTD-GFD joining site in vivo, and wherein a stop codon is positioned at the 3′ end of the GFD-encoding sequence. In an example of this group of variants (termed HRG-NTDXGFD), (1) the lysine residue found in the NTD-GFD joining sequence VKC is deleted or (preferably) substituted by another residue other than arginyl such as histidyl, alanyl, threonyl or seryl and (2) a stop codon is introduced in the sequence RCT or RCQ in place of cysteinyl, or threonyl (for HRG-α) or glutaminyl (for HRG-β).

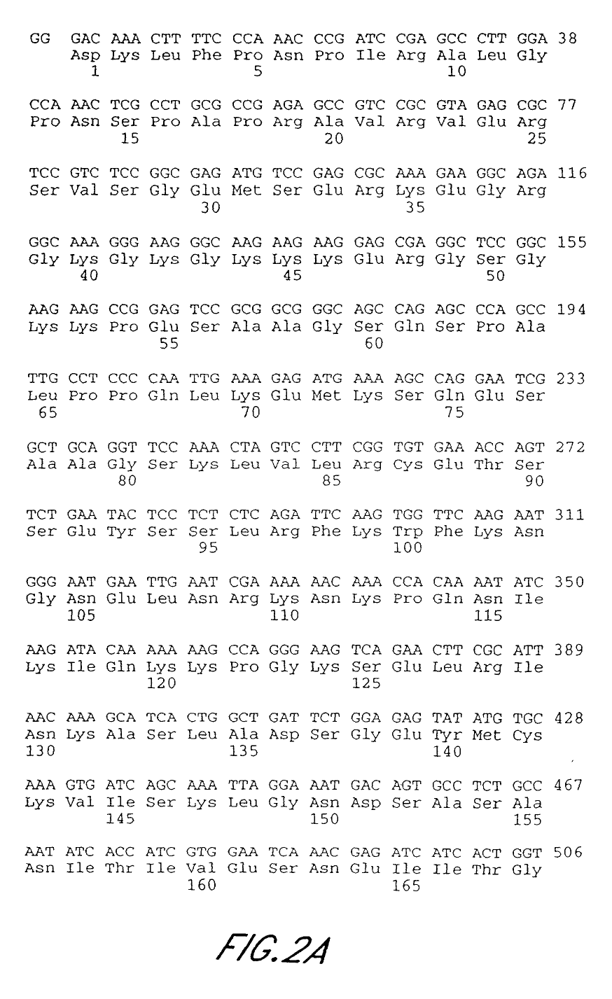

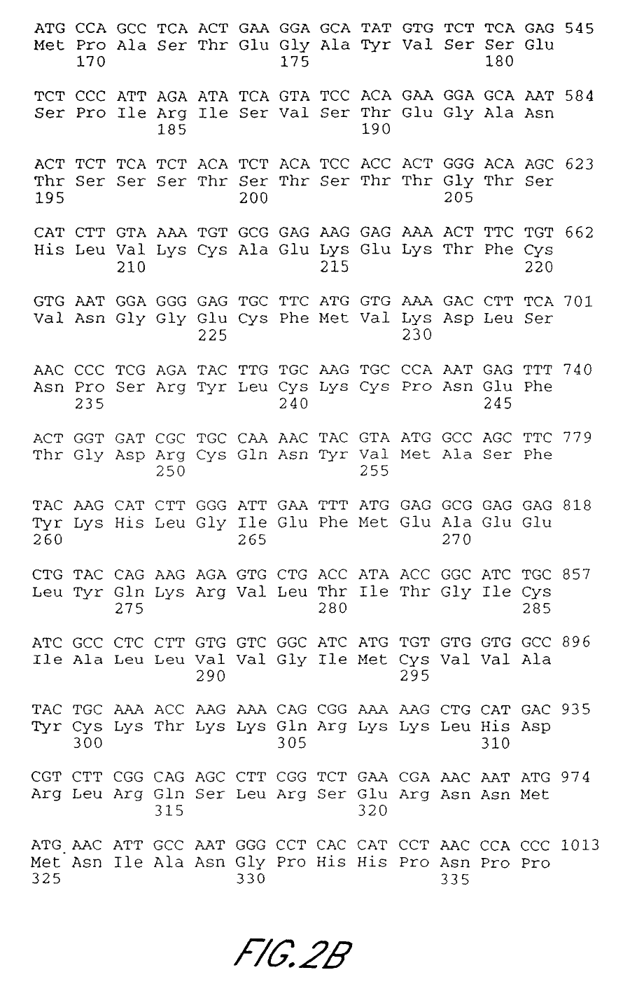

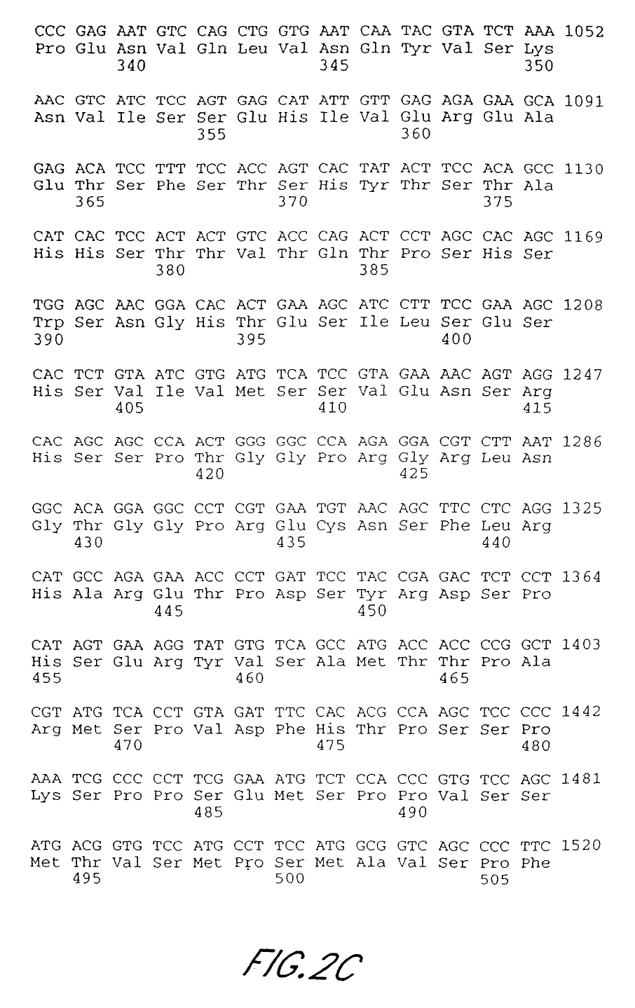

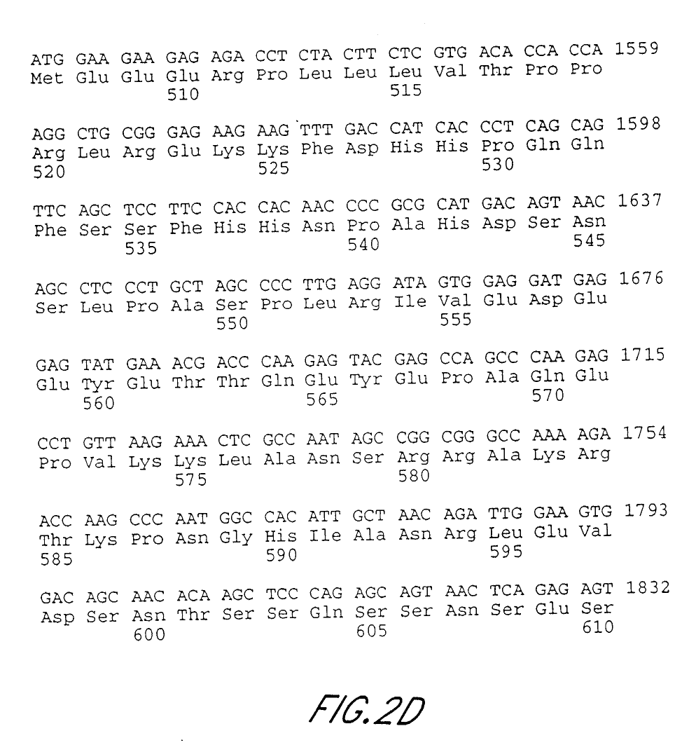

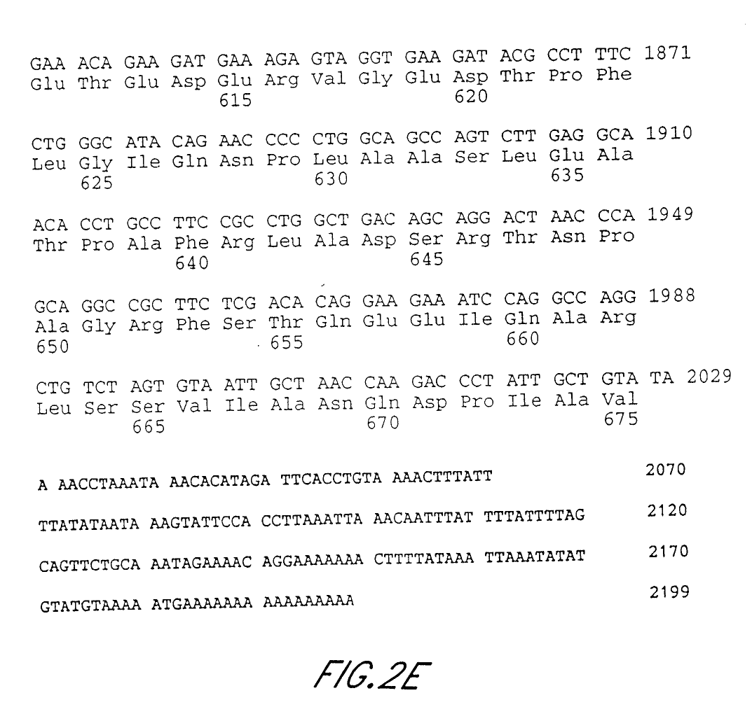

A preferred HRG-αligand with binding affinity to p185^{HER2} comprises amino acids 226-265 of FIG. 1A-D. This HRG-α ligand further may comprise up to an additional 1-20 amino acids preceding amino acid 226 and 1-20 amino acids following amino acid 265. A preferred HRG-β ligand with binding affinity to p185^{HER2} comprises amino acids 226-265 of FIG. 2A-E. This HRG-β ligand may comprise up to an additional 1-20 amino acids preceding amino acid 226 and 1-20 amino acids following amino acid 265.

As noted above, other heregulin sequences to be prepared in accordance with this invention are those of the GFD. These are synthesized in vitro or are produced in recombinant cell culture. These are produced most inexpensively in yeast or E.coli by secretion under the control of a heregulin-heterologous signal as described infra, although preparation in mammalian cells is also contemplated using a mammalian protein signal such as that of tPA, UK or a secreted viral protein. The GFD can be the sequence of a native heregulin or may be a variant thereof as described below. GFD sequences include those in which one or more residues from a member of the EGF family are substituted into or onto the GFD sequence.

An additional heregulin is one which contains the GFD and the sequence between the C-terminus of GFD and the N-terminus of the transmembrane domain (the later being termed the C-terminal cleavage domain or CTC). In this variant (HRG-GFD-CTC) the DNA start codon is present at the 5′ end of heregulin-heterologous signal sequence or adjacent the 5′ end of the GFD-encoding region, and a stop codon is found in place of one of the first about 1 to 3 extra-cellular domain (ECD) residues or first about 1-2 transmembrane region residues. In addition, in some HRG-GFD-CTC variants the codons are modified in the GFD-CTC proteolysis site by substitution, insertion or deletion. The GFD-CTC proteolysis site is the domain that contains the GFD C-terminal residue and about 5 residues N- and 5 residues C-terminal from this residue. It is known that Met-227 terminal and Val-229 terminal HRG-α-GFD are biologically active. The C-terminus for HRG-α-GFD may be Met-227, Lys-228, Val-229, Gln-230, Asn-231 or Gln-232, and for HRG-β-GFD may be Met-226, Ala-227, Ser-228, Phe-229, Trp-230, or Lys231/Ser231. The native C-terminus is determined readily by C-terminal sequencing, although it is not critical that HRG-GFD have the native terminus so long as the GFD sequence possesses the desired activity. In some embodiments of HRG-GFD-CTC variants, the amino acid change(s) in the CTC are screened for their ability to resist proteolysis in vitro and inhibit the protease responsible for generation of HRG-GFD.

HRG-ECD variants are made by providing a stop codon at the same location as for HRG-GFD-CTC variants. HRG-ECD may comprise any one or more of the variants described above in connection with its subfragments, e.g. the GFD-CTC variants containing CTC-proteolysis site modifications.

If it is desired to prepare the longer heregulin polypeptides and the 5′ or 3′ ends of the given heregulin are not described herein, it may be necessary to prepare nucleic acids in which the missing domains are supplied by homologous regions from more complete heregulin nucleic acids. Alternatively, the missing domains can be obtained by probing libraries using the DNAs disclosed in the Figures or fragments thereof.

A. Isolation of DNA Encoding Heregulin

The DNA encoding heregulin may be obtained from any cDNA library prepared from tissue believed to possess heregulin mRNA and to express it at a detectable level. HRG-α gene thus may be obtained from a genomic library. Similar procedures may be used for the isolation of other heregulin, such as HRG-β1, HRG-β2, or HRG-β3 encoding genes.

Libraries are screened with probes designed to identify the gene of interest or the protein encoded by it. For cDNA expression libraries, suitable probes include monoclonal or polyclonal antibodies that recognize and specifically bind to HRG-α; oligonucleotides of about 20-80 bases in length that encode known or suspected portions of HRG-α cDNA from the same or different species; and/or complementary or homologous cDNAs or fragments thereof that encode the same or a similar gene. Appropriate probes for screening genomic DNA libraries include, but are not limited to, oligonucleotides; cDNAs or fragments thereof that encode the same or a similar gene; and/or homologous genomic DNAs or fragments thereof. Screening the cDNA or genomic library with the selected probe may be conducted using standard procedures as described in chapters 10-12 of Sambrook et al., supra.

An alternative means to isolate the gene encoding HRG-α is to use polymerase chain reaction (PCR) methodology as described in section 14 of Sambrook et al., supra. This method requires the use of oligonucleotide probes that will hybridize to HRG-α. Strategies for selection of oligonucleotides are described below.

Another alternative method for obtaining the gene of interest is to chemically synthesize it using one of the methods described in Engels et al. (Agnew. Chem. Int. Ed. Engl., 28: 716-734,1989), specifically incorporated by reference. These methods include triester, phosphite, phosphoramidite and H-Phosphonate methods, PCR and other autoprimer methods, and oligonucleotide syntheses on solid supports. These methods may be used if the entire nucleic acid sequence of the gene is known, or the sequence of the nucleic acid complementary to the coding strand is available, or alternatively, if the target amino acid sequence is known, one may infer potential nucleic acid sequences using known and preferred coding residues for each amino acid residue.

A preferred method of practicing this invention is to use carefully selected oligonucleotide sequences to screen cDNA libraries from various tissues, preferably human breast, colon, salivary gland, placental, fetal, brain, and carcinoma cell lines. Other biological sources of DNA encoding an heregulin-like ligand include other mammals and birds. Among the preferred mammals are members of the following orders: bovine, ovine, equine, murine, and rodentia.

The oligonucleotide sequences selected as probes should be of sufficient length and sufficiently unambiguous that false positives are minimized. The actual nucleotide sequence(s) may, for example, be based on conserved or highly homologous nucleotide sequences or regions of HRG-α. The oligonucleotides may be degenerate at one or more positions. The use of degenerate oligonucleotides may be of particular importance where a library is screened from a species in which preferential codon usage in that species is not known. The oligonucleotide must be labeled such that it can be detected upon hybridization to DNA in the library being screened. The preferred method of labeling is to use ³²P-labeled ATP with polynucleotide kinase, as is well known in the art, to radiolabel the oligonucleotide. However, other methods may be used to label the oligonucleotide, including, but not limited to, biotinylation or enzyme labeling.

Of particular interest is HRG-α nucleic acid that encodes a full-length polypeptide. In some preferred embodiments, the nucleic acid sequence includes the native HRG-α signal sequence. Nucleic acid having all the protein coding sequence is obtained by screening selected cDNA or genomic libraries, and, if necessary, using conventional primer extension procedures as described in section 7.79 of Sambrook et al., supra, to detect precursors and processing intermediates of mRNA that may not have been reverse-transcribed into cDNA.

HRG-α encoding DNA of FIGS. 1A-1D may be used to isolate DNA encoding the analogous ligand from other animal species via hybridization employing the methods discussed above. The preferred animals are mammals, particularly bovine, ovine, equine, feline, canine and rodentia, and more specifically rats, mice and rabbits.

The EGF-like domain fragment HRG-β1 177-244 was amplified from vector pHL89 (which is described in Holmes et al., Science 256:1205-1210 (1992)) by PCR with primers having Nsil/Xbal-containing overhangs. The fragment was inserted into phagemid display vector pam-g3 by restriction digest-ligation at the same sites to generate construct pHRG2-g3 (177-244). pam-g3 was a derivative of phGHam-g3, which was designed for phage display of human growth hormone (hGH) and was described in Lowman et al., Biochemistry 30:10832-10838 (1991). pam-g3 was produced by removing the hGH gene present in phGHam-g3 and replacing this gene with a stuffer fragment, which provides space for cleavage at the restriction sites used for cloning. The HRG-β1 fragment was attached to residue 247 of pIII.

The HRG-β1 EGF-like domain expressed from the above-described construct is designated by removing the “p” and the “-g3” that appear in the name of the construct. Thus, the HRG-β1 EGF-like domain expressed from the pHRG2-g3 construct is designated “HRG2.” The domain was displayed monovalently on phage as a pIII fusion protein, as described by Bass et al., Proteins 8:309-314 (1990). Similarly, variants HRG-β1₁₄₇₋₂₂₇, HRG-β1₁₄₇₋₂₄₄, and HRG-β1₁₇₇₋₂₂₇ were prepared and express described above.

B. Amino Acid Sequence Variants of Heregulin

Amino acid sequence variants of heregulin are prepared by introducing appropriate nucleotide changes into heregulin DNA, or by in vitro synthesis of the desired heregulin polypeptide. Such variants include, for example, deletions from, or insertions or substitutions of, residues within the amino acid sequence shown for human heregulin sequences. Any combination of deletion, insertion, and substitution can be made to arrive at the final construct, provided that the final construct possesses the desired characteristics. Excluded from the scope of this invention are heregulin variants or polypeptide sequences that are not novel and unobvious over the prior art. The amino acid changes also may alter post-translational processes of HRG-α, such as changing the number or position of glycosylation sites, altering the membrane anchoring characteristics, altering the intra-cellular location of heregulin by inserting, deleting, or otherwise affecting the leader sequence of the native heregulin, or modifying its susceptibility to proteolytic cleavage.

The heregulin sequence may be proteolytically processed to create a number of heregulin fragments. HRG-GFD sequences of HRG-α all contain the amino acid sequence between HRG-α cysteine 226 and cysteine 265. The amino terminus of HRG-α fragment may result from the cleavage of any peptide bond between alanine 1 and cysteine 226, preferably adjacent to an arginine, lysine, valine, or methionine, and most preferably between methionine 45 and serine 46. The carboxy terminus of HRG-α fragment may result from the cleavage of any peptide bond between cysteine 265, preferably adjacent to an arginine, lysine, valine, or methionine, and most preferably between lysine 272 and valine 273, between lysine 278 and alanine 279, or between lysine 285 and arginine 286. The resulting HRG-α ligands resulting from such proteolytic processing are the preferred ligands.

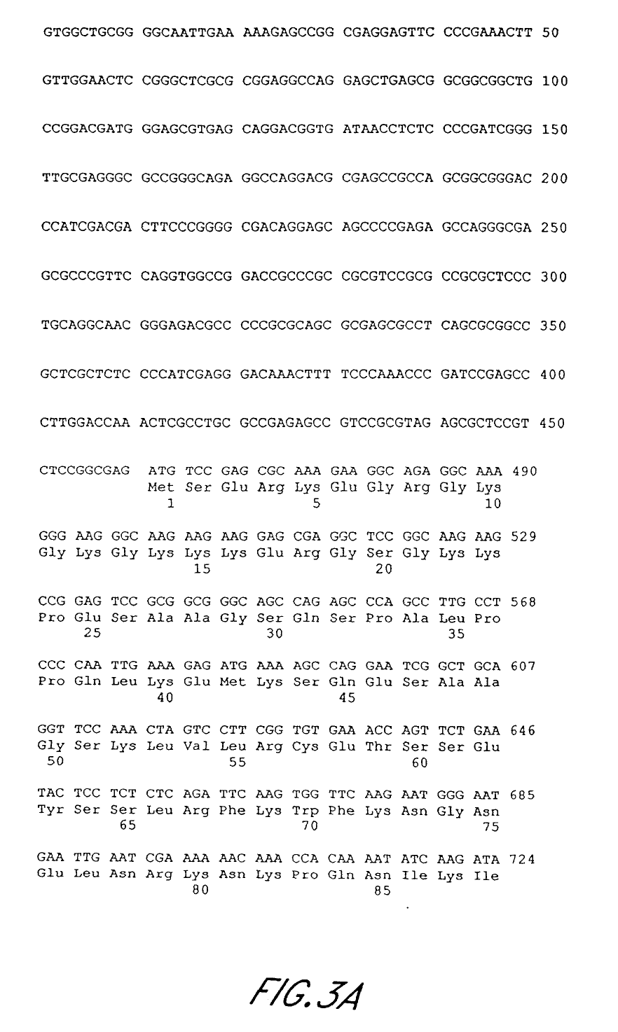

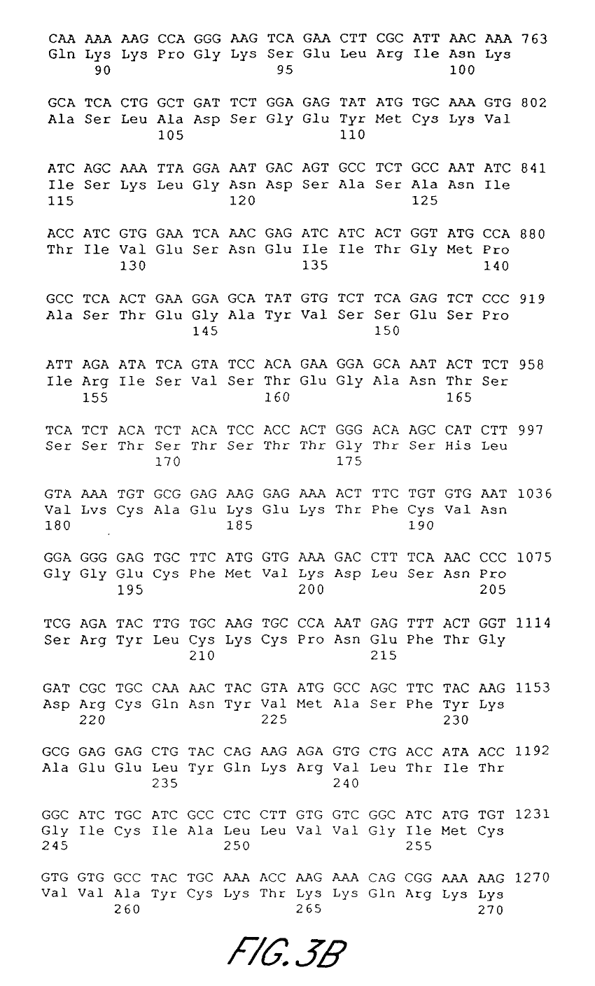

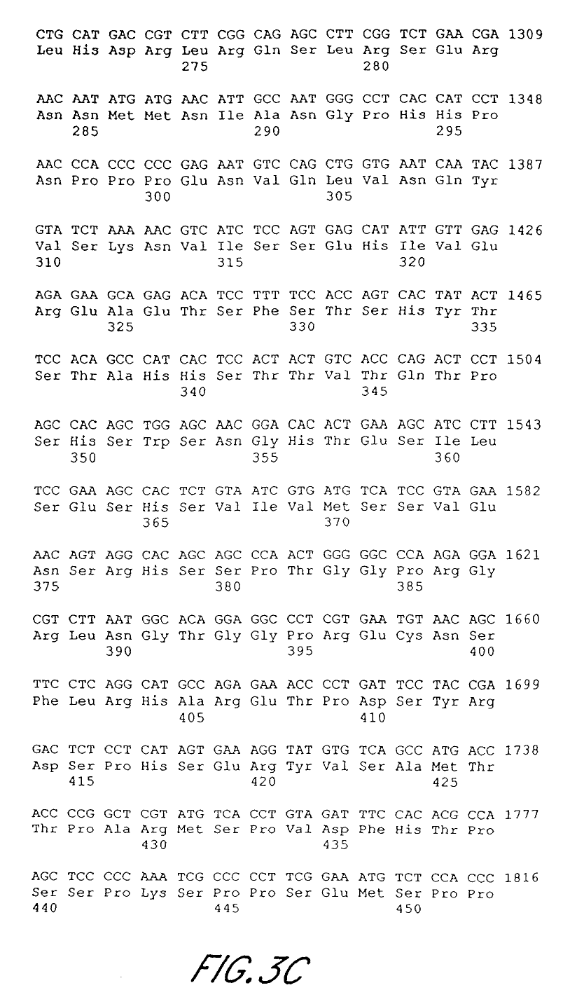

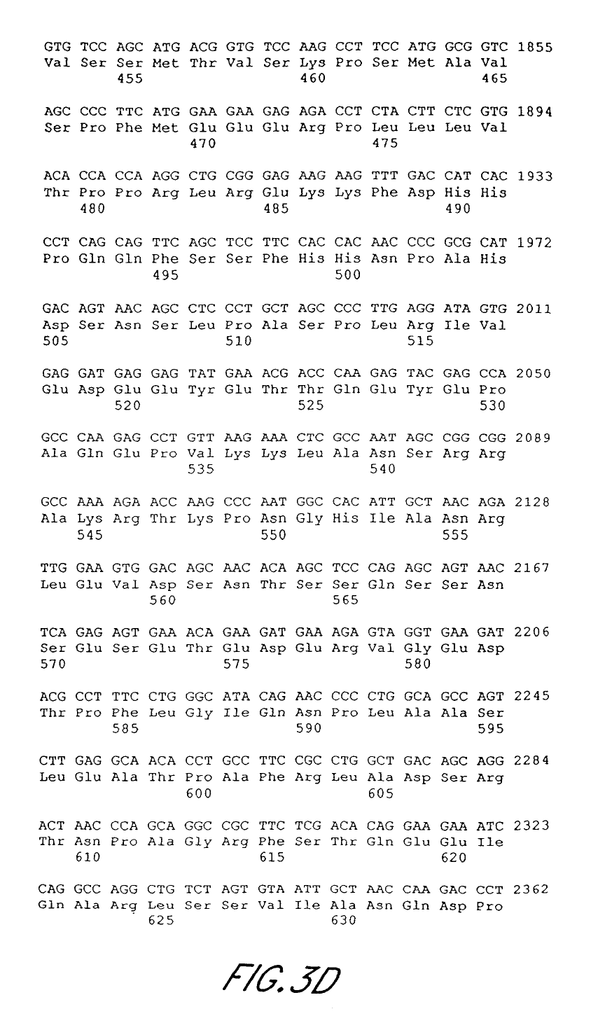

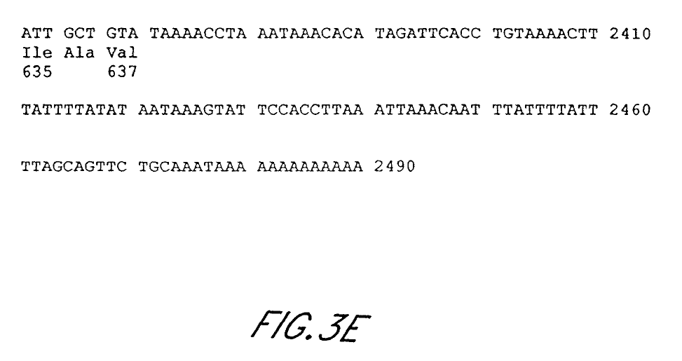

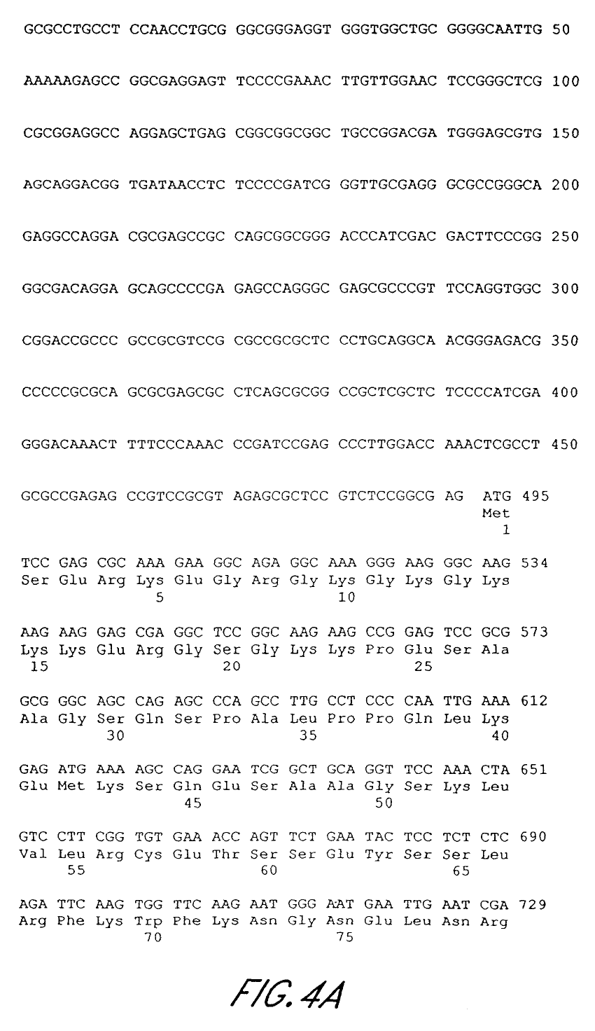

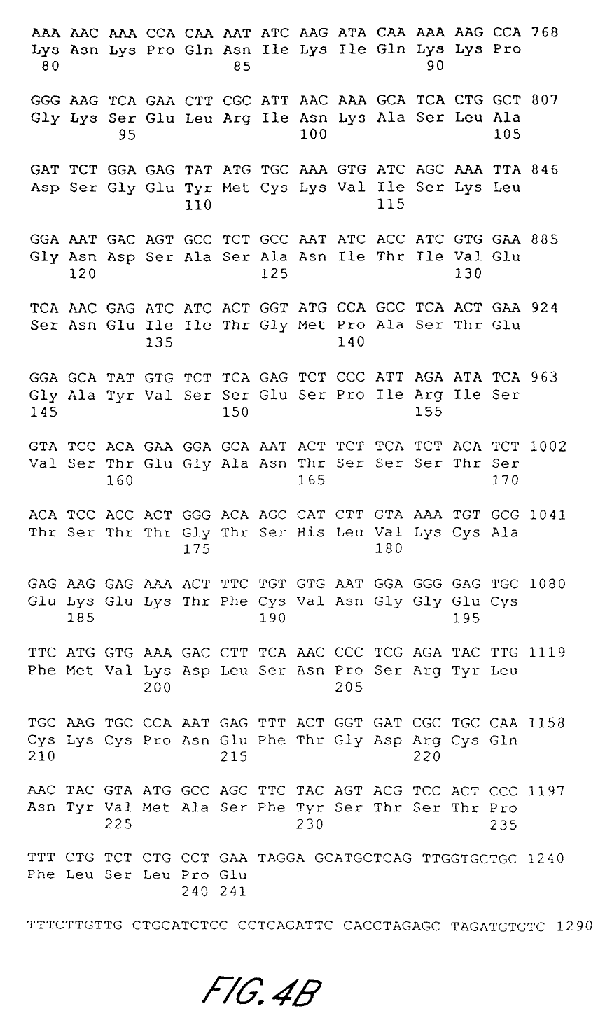

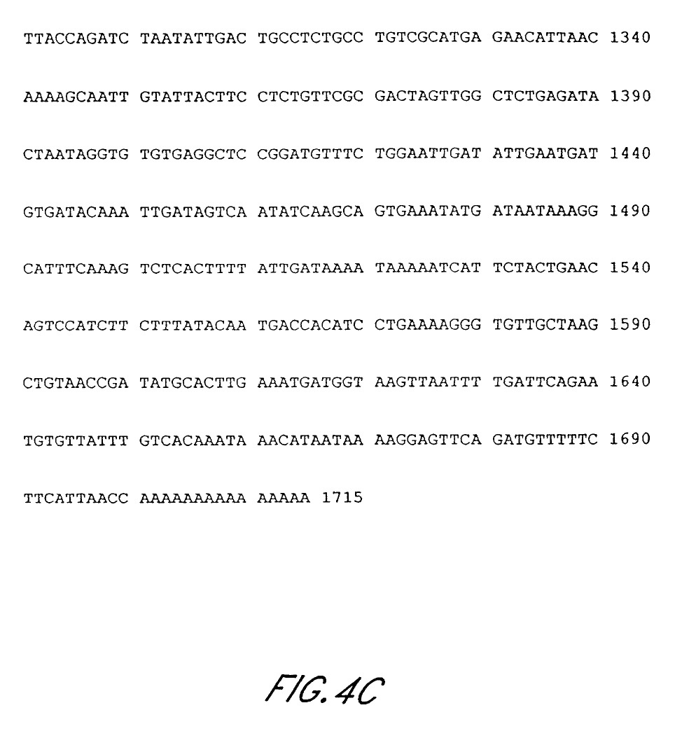

HRG-β-GFD's are analogous to those discussed above for HRG-α-GFD's. Each HRG-β-GFD contains the polypeptide segment from cysteine 212 to cysteine 251 of FIG. 2A-E. The amino terminus of HRG-β1 fragment may result from the cleavage of any peptide bond between alanine 1 and cysteine 212, preferably adjacent to an arginine, lysine, valine, or methionine, and most preferably between methionine 31 and serine 32. The carboxy terminus of HRG-β1 fragment may result from the cleavage of any peptide bond between cysteine 251 of FIG. 2A-2E, preferably adjacent to an arginine, lysine, valine, or methionine, and most preferably between valine 255 and methionine 256, between lysine 261 and histidine 262, between lysine 276 and alanine 277, or between lysine 301 and thrionine 302. The resulting HRG-β1 ligands resulting from such proteolytic processing are among the preferred ligands. Similarly, processing to produce preferred fragment ligands of HRG-β2 based upon the FIG. 3A-3E and HRG-β3 based upon FIG. 4A-4C may be accomplished by cleaving heregulin sequences of FIGS. 3A-3E and 4A-4C preferably adjacent to an arginine, lysine, valine or methionine.

In designing amino acid sequence variants of heregulin, the location of the mutation site and the nature of the mutation will depend on heregulin characteristic(s) to be modified. The sites for mutation can be modified individually or in series, e.g., by (1) substituting first with conservative amino acid choices and then with more radical selections depending upon the results achieved, (2) deleting the target residue, or (3) inserting residues of other receptor ligands adjacent to the located site.

A useful method for identification of certain residues or regions of heregulin polypeptide that are preferred locations for mutagenesis is called “alanine scanning mutagenesis” as described by Cunningham and Wells (Science, 244: 1081-1085, 1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with the surrounding aqueous environment in or outside the cell. Those domains demonstrating functional sensitivity to the substitutions then are refined by introducing further or other variants at or for the sites of substitution. Thus, while the site for introducing an amino acid sequence variation is predetermined, the nature of the mutation per se need not be predetermined. For example, to optimize the performance of a mutation at a given site, ala scanning or random mutagenesis may be conducted at the target codon or region and the expressed heregulin variants are screened for the optimal combination of desired activity.

There are two principal variables in the construction of amino acid sequence variants: the location of the mutation site and the nature of the mutation. These are variants from heregulin sequence, and may represent naturally occurring alleles (which will not require manipulation of heregulin DNA) or predetermined mutant forms made by mutating the DNA, either to arrive at an allele or a variant not found in nature. In general, the location and nature of the mutation chosen will depend upon heregulin characteristic to be modified. Obviously, such variations that, for example, convert heregulin into a known receptor ligand, are not included within the scope of this invention, nor are any other heregulin variants or polypeptide sequences that are not novel and unobvious over the prior art.

Amino acid sequence deletions generally range from about 1 to 30 residues, more preferably about 1 to 10 residues, and typically about 1 to 5 are contiguous. Deletions may be introduced into regions of low homology with other EGF family precursors to modify the activity of heregulin. Deletions from heregulin in areas of substantial homology with other EGF family sequences will be more likely to modify the biological activity of heregulin more significantly. The number of consecutive deletions will be selected so as to preserve the tertiary structure of heregulin in the affected domain, e.g., cysteine crosslinking, beta-pleated sheet or alpha helix.

Amino acid sequence insertions include amino- and/or carboxyl-terminal fusions ranging in length from one residue to polypeptides containing a hundred or more residues, as well as intrasequence insertions of single or multiple amino acid residues. Intrasequence insertions (i.e., insertions within heregulin sequence) may range generally from about 1 to 10 residues, more preferably 1 to 5, and most preferably 1 to 3. Examples of terminal insertions include heregulin with an N-terminal methionyl residue (an artifact of the direct expression of heregulin in bacterial recombinant cell culture), and fusion of a heterologous N-terminal signal sequence to the N-terminus of heregulin to facilitate the secretion of mature heregulin from recombinant host cells. Such signal sequences generally will be obtained from, and thus be homologous to, the intended host cell species. Suitable sequences include STII, tPA or Ipp for E. coli, alpha factor for yeast, and viral signals such as herpes gD for mammalian cells.

Other insertional variants of heregulin include the fusion to the N- or C-terminus of heregulin of an immunogenic polypeptide, e.g., bacterial polypeptides such as beta-lactamase or an enzyme encoded by the E. coli trp locus, or yeast protein, bovine serum albumin, and chemotactic polypeptides. C-terminal fusions of heregulin-ECD with proteins having a long half-life such as immunoglobulin constant regions (or other immunoglobulin regions), albumin, or ferritin, as described in WO 89/02922, published Apr. 6, 1989 are contemplated.

Another group of variants are amino acid substitution variants. These variants have at least one amino acid residue in heregulin molecule removed and a different residue inserted in its place. The sites of greatest interest for substitutional mutagenesis include sites identified as the active site(s) of heregulin, and sites where the amino acids found in heregulin ligands from various species are substantially different in terms of side-chain bulk, charge, and/or hydrophobicity. A likely sub-domain of HRG-GFD having biological activity as a growth factor is the C-terminal segment, in particular within the sequence about from glycine 218 to valine 226 (HRG-α), and glycine 218 to lysine 228/serine 228 (HRG-β) based upon analogy to the EGF sub-sequence found to have EGF activity.

Other sites of interest are those in which particular residues of heregulin-like ligands obtained from various species are identical. These positions may be important for the biological activity of heregulin. These sites, especially those falling within a sequence of at least three other identically conserved sites, are substituted in a relatively conservative manner. Such conservative substitutions are shown in Table 1 under the heading of “preferred substitutions”. If such substitutions result in a change in biological activity, then more substantial changes, denominated exemplary substitutions in Table 1, or as further described below in reference to amino acid classes, are introduced and the products screened.

TABLE 1

| Original | | Exemplary | Preferred |
|---|---|---|---|
| Residue | | Substitutions | Substitutions |
| Ala | (A) | val; leu; ile | val |
| Arg | (R) | lys; gln; asn | lys |
| Asn | (N) | gln; his; lys; arg | gln |
| Asp | (D) | glu | glu |
| Cys | (C) | ser | ser |
| Gln | (Q) | asn | asn |
| Glu | (E) | asp | asp |
| Gly | (G) | pro | pro |
| His | (H) | asn; gln; lys; arg | arg |
| Ile | (I) | leu; val; met; ala; phe; | leu |
| | | norleucine | |
| Leu | (L) | norleucine; ile; val; | ile |
| | | met; ala; phe | |
| Lys | (K) | arg; gln; asn | arg |
| Met | (M) | leu; phe; ile | leu |
| Phe | (F) | leu; val; ile; ala | leu |
| Pro | (P) | gly | gly |
| Ser | (S) | thr | thr |
| Thr | (T) | ser | ser |
| Trp | (W) | tyr | tyr |
| Tyr | (Y) | trp; phe; thr; ser | phe |
| Val | (V) | ile; leu; met; phe; | leu |
| | | ala; norleucine | |

Substantial modifications in function or immunological identity of heregulin are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side chain properties:

1) hydrophobic: norleucine, met, ala, val, leu, ile;

2) neutral hydrophilic: cys, ser, thr;

3) acidic: asp, glu;

4) basic: asn, gin, his, lys, arg;

5) residues that influence chain orientation: gly, pro; and

6) aromatic: trp, tyr, phe.

Non-conservative substitutions will entail exchanging a member of one of these classes for another. Such substituted residues may be introduced into regions of heregulin that are homologous with other receptor ligands, or, more preferably, into the non-homologous regions of the molecule.

In one embodiment of the invention, it is desirable to inactivate one or more protease cleavage sites that are present in the molecule. These sites are identified by inspection of the encoded amino acid sequence. Where protease cleavage sites are identified, they are rendered inactive to proteolytic cleavage by substituting the targeted residue with another residue, preferably a basic residue such as glutamine or a hydrophylic residue such as serine; by deleting the residue; or by inserting a prolyl residue immediately after the residue.

In another embodiment, any methionyl residue other than the starting methionyl residue of the signal sequence, or any residue located within about three residues N- or C-terminal to each such methionyl residue, is substituted by another residue (preferably in accord with Table 1) or deleted. Alternatively, about 1-3 residues are inserted adjacent to such sites.

Any cysteine residues not involved in maintaining the proper conformation of heregulin also may be substituted, generally with serine, to improve the oxidative stability of the molecule and prevent aberrant crosslinking.

Sites particularly suited for substitutions, deletions or insertions, or use as fragments, include, numbered from the N-terminus of HRG-α of FIG. 1A-1D:

1) potential glycosaminoglycan addition sites at the serine-glycine dipeptides at 42-43, 64-65, 151-152;

2) potential asparagine-linked glycosylation at positions 164, 170, 208 and 437, sites (NDS) 164-166, (NIT) 170-172, (NTS) 208-210, and NTS (609-611);

3) potential O-glycosylation in a cluster of serine and threonine at 209-218;

4) cysteines at 226, 234, 240, 254, 256 and 265;

5) transmembrane domain at 287-309;

6) loop 1 delineated by cysteines 226 and 240;

7) loop 2 delineated by cysteines 234 and 254;

8) loop 3 delineated by cysteines 256 and 265; and

9) potential protease processing sites at 2-3, 8-9, 23-24, 33-34, 36-37, 45-46, 48-49, 62-63, 66-67, 86-87, 110-111, 123-124, 134-135, 142-143, 272-273, 278-279 and 285-286;

Analogous regions in HRG-β1 may be determined by reference to its' sequence. The analogous HRG-β1 amino acids may be mutated or modified as discussed above for HRG-α. Analogous regions in HRG-β2 may also be determined by reference to its' sequence. The analogous HRG-β2 amino acids may be mutated or modified as discussed above for HRG-α or HRG-β1. Analogous regions in HRG-β3 may be determined by reference to its' sequence. Further, the analogous HRG-β3 amino acids may be mutated or modified as discussed above for HRG-α, HRG-β1, or HRG-β2.

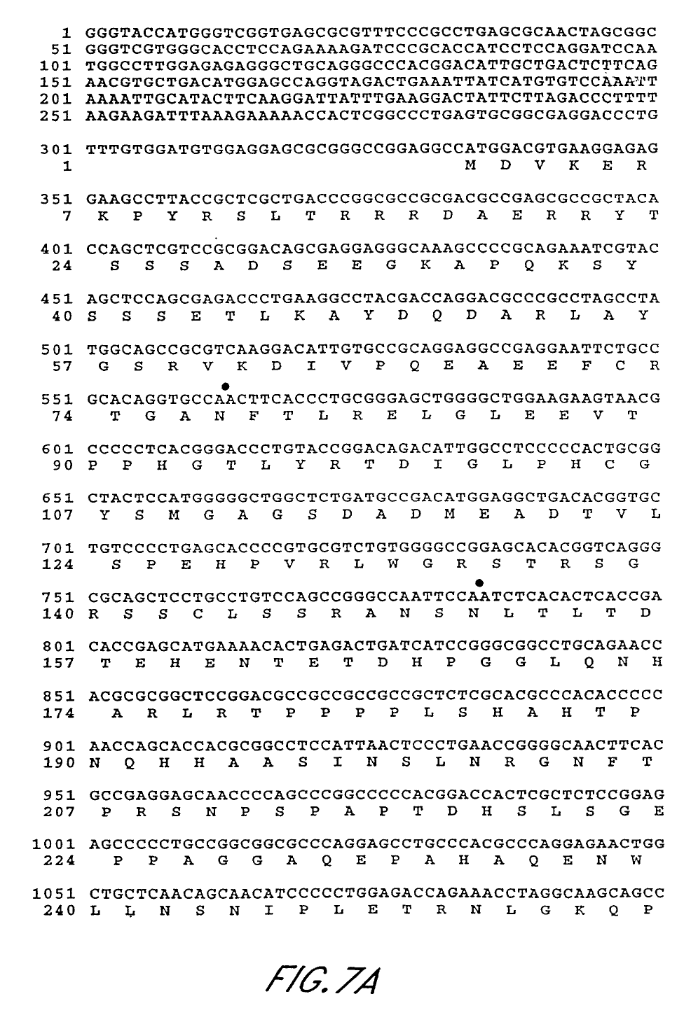

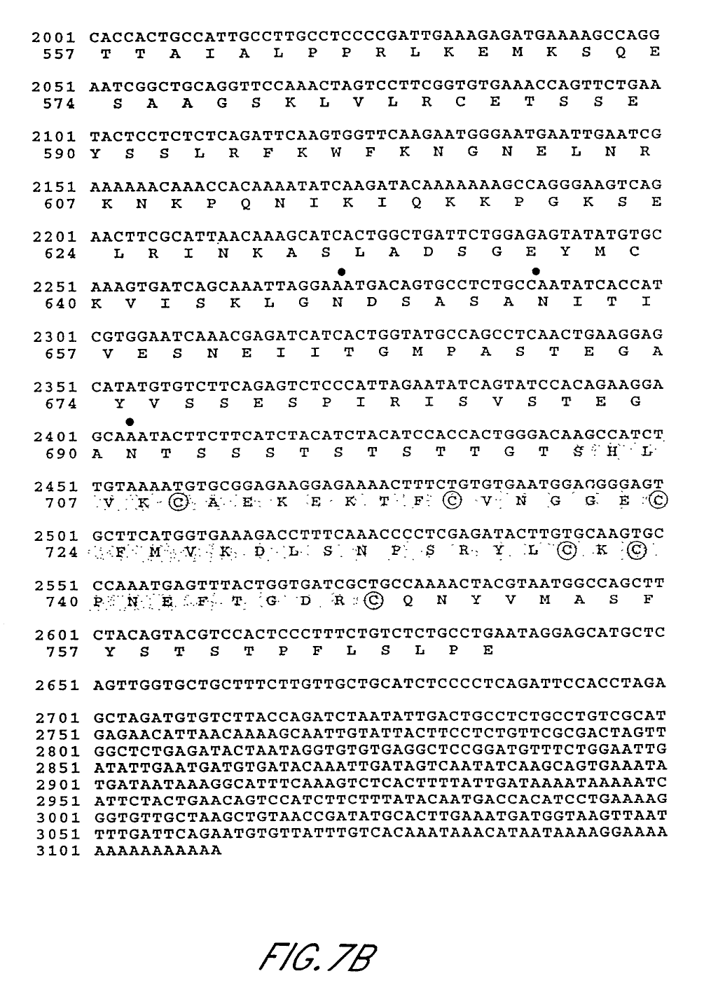

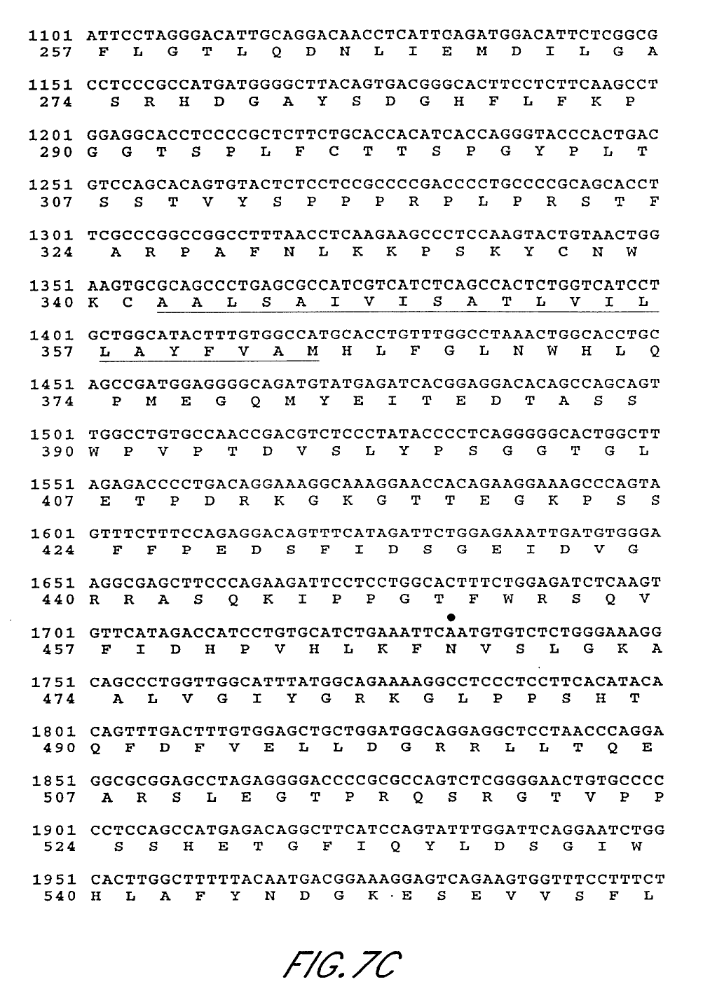

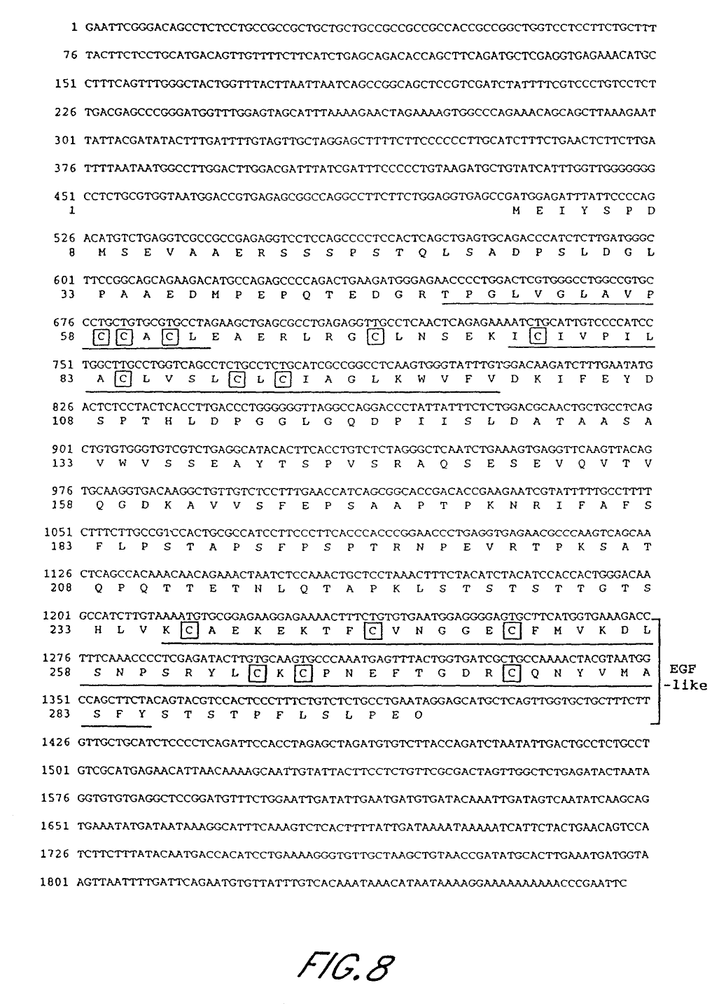

Another heregulin variant is or gamma-heregulin. -HRG is any polypeptide sequence that possesses at least one biological property of native sequence -HRG having SEQ ID NO:11. The biological property of this variant is the same as for heregulin noted above. This variant encompasses not only the polypeptide isolated from a native -HRG source such as human MDA-MB-175 cells or from another source, such as another animal species, but also the polypeptide prepared by recombinant or synthetic methods. It also includes variant forms including functional derivatives, allelic variants, naturally occurring isoforms and analogues thereof. Sometimes the -HRG is “native -HRG” which refers to endogenous -HRG polypeptide which has been isolated from a mammal. The -HRG can also be “native sequence -HRG ” insofar as it has the same amino acid sequence as a native -HRG (e.g. human -HRG shown in FIG. 7A-7C). Amino acid sequence variants of the native sequence are prepared by introducing appropriate nucleotide changes into the native sequence DNA, or by in vitro synthesis of the desired polypeptide. Such variants include, for example, deletions from, or insertions or substitutions of, residues within the amino acid sequence shown for the human protein in FIG. 7A-7C as generally described agove for other heregulin. Any combination of deletion, insertion, and substitution is made to arrive at the final construct, provided that the final construct possesses the desired characteristics. The amino acid changes also may alter post-translational processes of the native sequence, such as changing the number or position of O-linked glycosylation sites.

Additional variants include polypeptides in which the variant has an amino acid substitution at a selected residue corresponding to a residue of 645-amino acid native human heregulin-β1 selected from:

S177, H178, L179, V180, K181, E184, E186, K187, T188, V191, N192, G193, G194, E195, M198, V199, K200, D201, N204, P205, S206, R207, Y208, L209, K211, P213, N214, E215, T217, G218, D219, Q222, N223, Y224, S228, and F229.

In a variation of this embodiment, the amino acid substitution is not a replacement of the selected residue with an epidermal growth factor (EGF) residue corresponding to the selected residue.

Other heregulin-β1 variants include an amino acid substitution selected from:

S177W; H178S, E, R, or A; V180Q, I or E; K181P or A; A183G; E184V, W, K, R, G, or N; K185E, S, Q, or G; E186R; K187E or A; T188Q; E195Q; F197Y; M198R or K; K200R; D201T or I; P205T or Y; S206K, H, G, P, or R; R207Y; Y208R or L; L209M or G; K211R; P213S, T, N, or K; N214L, K, S, or E; F216M; N223H or W; and M226I.

In a variation of this embodiment, the heregulin variant includes sets of amino acid substitutions selected from this group.

In addition to including one or more of the amino acid substitutions disclosed herein, the heregulin variant can have one or more other modifications, such as an amino acid substitution, an insertion of at least one amino acid, a deletion of at least one amino acid, or a chemical modification. For example, the invention provides a heregulin variant that is a fragment. In a variation of this embodiment, the fragment includes residues corresponding to a portion of human heregulin-β1 extending from about residue 175 to about residue 230 (i.e., the EGF-like domain). For example, the fragment can extend from residue 177 to residue 244 and may be prepared by recombinant techniques (rHRGβ1-177-244).

DNA encoding amino acid sequence variants of heregulin is prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of heregulin. These techniques may utilize heregulin nucleic acid (DNA or RNA), or nucleic acid complementary to heregulin nucleic acid.

Oligonucleotide-mediated mutagenesis is a preferred method for preparing substitution, deletion, and insertion variants of heregulin DNA. This technique is well known in the art as described by Adelman et al., DNA, 2: 183 (1983). Briefly, heregulin DNA is altered by hybridizing an oligonucleotide encoding the desired mutation to a DNA template, where the template is the single-stranded form of a plasmid or bacteriophage containing the unaltered or native DNA sequence of heregulin. After hybridization, a DNA polymerase is used to synthesize an entire second complementary strand of the template that will thus incorporate the oligonucleotide primer, and will code for the selected alteration in heregulin DNA.

Generally, oligonucleotides of at least 25 nucleotides in length are used. An optimal oligonucleotide will have 12 to 15 nucleotides that are completely complementary to the template on either side of the nucleotide(s) coding for the mutation. This ensures that the oligonucleotide will hybridize properly to the single-stranded DNA template molecule. The oligonucleotides are readily synthesized using techniques known in the art such as that described by Crea et al. (Proc. Natl. Acad. Sci. USA, 75: 5765, 1978).

Single-stranded DNA template may also be generated by denaturing double-stranded plasmid (or other) DNA using standard techniques.

For alteration of the native DNA sequence (to generate amino acid sequence variants, for example), the oligonucleotide is hybridized to the single-stranded template under suitable hybridization conditions. A DNA polymerizing enzyme, usually the Klenow fragment of DNA polymerase I, is then added to synthesize the complementary strand of the template using the oligonucleotide as a primer for synthesis. A heteroduplex molecule is thus formed such that one strand of DNA encodes the mutated form of heregutin, and the other strand (the original template) encodes the native, unaltered sequence of heregulin. This heteroduplex molecule is then transformed into a suitable host cell, usually a prokaryote such as E. coli JM101. After the cells are grown, they are plated onto agarose plates and screened using the oligonucleotide primer radiolabeled with ³²P-phosphate to identify the bacterial colonies that contain the mutated DNA. The mutated region is then removed and placed in an appropriate vector for protein production, generally an expression vector of the type typically employed for transformation of an appropriate host.

The method described immediately above may be modified such that a homoduplex molecule is created wherein both strands of the plasmid contain the mutation(s). The modifications are as follows: the single-stranded oligonucleotide is annealed to the single-stranded template as described above. A mixture of three deoxyribonucleotides, deoxyriboadenosine (dATP), deoxyriboguanosine (dGTP), and deoxyribothymidine (dTTP), is combined with a modified thio-deoxyribocytosine called dCTP-(aS) (which can be obtained from Amersham Corporation). This mixture is added to the template-oligonucleotide complex. Upon addition of DNA polymerase to this mixture, a strand of DNA identical to the template except for the mutated bases is generated. In addition, this new strand of DNA will contain dCTP-(aS) instead of dCTP, which serves to protect it from restriction endonuclease digestion. After the template strand of the double-stranded heteroduplex is nicked with an appropriate restriction enzyme, the template strand can be digested with ExoIII nuclease or another appropriate nuclease past the region that contains the site(s) to be mutagenized. The reaction is then stopped to leave a molecule that is only partially single-stranded. A complete double-stranded DNA homoduplex is then formed using DNA polymerase in the presence of all four deoxyribonucleotide triphosphates, ATP, and DNA ligase. This homoduplex molecule can then be transformed into a suitable host cell such as E. coli JM101, as described above.

DNA encoding heregulin mutants with more than one amino acid to be substituted may be generated in one of several ways. If the amino acids are located close together in the polypeptide chain, they may be mutated simultaneously using one oligonucleotide that codes for all of the desired amino acid substitutions. If, however, the amino acids are located some distance from each other (separated by more than about ten amino acids), it is more difficult to generate a single oligonucleotide that encodes all of the desired changes. Instead, one of two alternative methods may be employed.

In the first method, a separate oligonucleotide is generated for each amino acid to be substituted. The oligonucleotides are then annealed to the single-stranded template DNA simultaneously, and the second strand of DNA that is synthesized from the template will encode all of the desired amino acid substitutions.

The alternative method involves two or more rounds of mutagenesis to produce the desired mutant. The first round is as described for the single mutants: wild-type DNA is used for the template, an oligonucleotide encoding the first desired amino acid substitution(s) is annealed to this template, and the heteroduplex DNA molecule is then generated. The second round of mutagenesis utilizes the mutated DNA produced in the first round of mutagenesis as the template. Thus, this template already contains one or more mutations. The oligonucleotide encoding the additional desired amino acid substitution(s) is then annealed to this template, and the resulting strand of DNA now encodes mutations from both the first and second rounds of mutagenesis. This resultant DNA can be used as a template in a third round of mutagenesis, and so on.

PCR mutagenesis is also suitable for making amino acid variants of heregulin. While the following discussion refers to DNA, it is understood that the technique also finds application with RNA. The PCR technique generally refers to the following procedure (see Erlich, supra, the chapter by R. Higuchi, p. 61-70). When small amounts of template DNA are used as starting material in a PCR, primers that differ slightly in sequence from the corresponding region in a template DNA can be used to generate relatively large quantities of a specific DNA fragment that differs from the template sequence only at the positions where the primers differ from the template. For introduction of a mutation into a plasmid DNA, one of the primers is designed to overlap the position of the mutation and to contain the mutation; the sequence of the other primer must be identical to a stretch of sequence of the opposite strand of the plasmid, but this sequence can be located anywhere along the plasmid DNA. It is preferred, however, that the sequence of the second primer is located within 200 nucleotides from that of the first, such that in the end the entire amplified region of DNA bounded by the primers can be easily sequenced. PCR amplification using a primer pair like the one just described results in a population of DNA fragments that differ at the position of the mutation specified by the primer, and possibly at other positions, as template copying is somewhat error-prone.

If the ratio of template to product material is extremely low, the vast majority of product DNA fragments incorporate the desired mutation(s). This product material is used to replace the corresponding region in the plasmid that served as PCR template using standard DNA technology. Mutations at separate positions can be introduced simultaneously by either using a mutant second primer, or performing a second PCR with different mutant primers and ligating the two resulting PCR fragments simultaneously to the vector fragment in a three (or more)-part ligation.

In a specific example of PCR mutagenesis, template plasmid DNA (1 mg) is linearized by digestion with a restriction endonuclease that has a unique recognition site in the plasmid DNA outside of the region to be amplified. Of this material, 100 ng is added to a PCR mixture containing PCR buffer, which contains the four deoxynucleotide tri-phosphates and is included in the GENEAMP kits (obtained from Perkin-Elmer Cetus, Norwalk, Conn. and Emeryville, Calif.), and 25 pmole of each oligonucleotide primer, to a final volume of 50 ml. The reaction mixture is overlayed with 35 ml mineral oil. The reaction is denatured for 5 minutes at 100° C., placed briefly on ice, and then 1 ml Thermus aquaticus (Taq) DNA polymerase (5 units/ml, purchased from Perkin-Elmer Cetus, Norwalk, Conn. and Emeryville, Calif.) is added below the mineral oil layer. The reaction mixture is then inserted into a DNA Thermal Cycler (purchased from Perkin-Elmer Cetus) programmed as follows:

2 min. 55° C.,

30 sec. 72° C., then 19 cycles of the following:

30 sec. 94° C.,

30 sec. 55° C., and

30 sec. 72° C.

At the end of the program, the reaction vial is removed from the thermal cycler and the aqueous phase transferred to a new vial, extracted with phenol/chloroform (50:50:vol), and ethanol precipitated, and the DNA is recovered by standard procedures. This material is subsequently subjected to the appropriate treatments for insertion into a vector.

Another method for preparing variants, cassette mutagenesis, is based on the technique described by Wells et al. (Gene, 34: 315, 1985). The starting material is the plasmid (or other vector) comprising heregulin DNA to be mutated. The codon(s) in heregulin DNA to be mutated are identified. There must be a unique restriction endonuclease site on each side of the identified mutation site(s). If no such restriction sites exist, they may be generated using the above-described oligonucleotide-mediated mutagenesis method to introduce them at appropriate locations in heregulin DNA. After the restriction sites have been introduced into the plasmid, the plasmid is cut at these sites to linearize it. A double-stranded oligonucleotide encoding the sequence of the DNA between the restriction sites but containing the desired mutation(s) is synthesized using standard procedures. The two strands are synthesized separately and then hybridized together using standard techniques. This double-stranded oligonucleotide is referred to as the cassette. This cassette is designed to have 3′ and 5′ ends that are compatible with the ends of the linearized plasmid, such that it can be directly ligated to the plasmid. This plasmid now contains the mutated heregulin DNA sequence.

C. Insertion of DNA into a Cloning Vehicle

The cDNA or genomic DNA encoding native or variant heregulin is inserted into a replicable vector for further cloning (amplification of the DNA) or for expression. Many vectors are available, and selection of the appropriate vector will depend on 1) whether it is to be used for DNA amplification or for DNA expression, 2) the size of the DNA to be inserted into the vector, and 3) the host cell to be transformed with the vector. Each vector contains various components depending on its function (amplification of DNA or expression of DNA) and the host cell for which it is compatible. The vector components generally include, but are not limited to, one or more of the following: a signal sequence, an origin of replication, one or more marker genes, an enhancer element, a promoter, and a transcription termination sequence.

(i) Signal Sequence Component

In general, the signal sequence may be a component of the vector, or it may be a part of heregulin DNA that is inserted into the vector. The native heregulin DNA is believed to encode a signal sequence at the amino terminus (5′ end of the DNA encoding heregulin) of the polypeptide that is cleaved during post-translational processing of the polypeptide to form the mature heregulin polypeptide ligand that binds to the HER2/HER3 receptor, although a conventional signal structure is not apparent. Native heregulin is, secreted from the cell but remains lodged in the membrane because it contains a transmembrane domain and a cytoplasmic region in the carboxyl terminal region of the polypeptide. Thus, in a secreted, soluble version of heregulin the carboxyl terminal domain of the molecule, including the transmembrane domain, is ordinarily deleted. This truncated variant heregulin polypeptide may be secreted from the cell, provided that the DNA encoding the truncated variant encodes a signal sequence recognized by the host.

Heregulin of this invention may be expressed not only directly, but also as a fusion with a heterologous polypeptide, preferably a signal sequence or other polypeptide having a specific cleavage site at the N- and/or C-terminus of the mature protein or polypeptide. In general, the signal sequence may be a component of the vector, or it may be a part of heregulin DNA that is inserted into the vector. Included within the scope of this invention are heregulin with the native signal sequence deleted and replaced with a heterologous signal sequence. The heterologous signal sequence selected should be one that is recognized and processed, i.e., cleaved by a signal peptidase, by the host cell. For prokaryotic host cells that do not recognize and process the native heregulin signal sequence, the signal sequence is substituted by a prokaryotic signal sequence selected, for example, from the group of the alkaline phosphatase, penicillinase, Ipp, or heat-stable enterotoxin II leaders. For yeast secretion the native heregulin signal sequence may be substituted by the yeast invertase, alpha factor, or acid phosphatase leaders. In mammalian cell expression the native signal sequence is satisfactory, although other mammalian signal sequences may be suitable.

(ii) Origin of Replication Component

Both expression and cloning vectors generally contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2 m plasmid origin is suitable for yeast, and various viral origins (SV40, polyoma, adenovirus, VSV or BPV) are useful for cloning vectors in mammalian cells. Generally, the origin of replication component is not needed for mammalian expression vectors (the SV40 origin may typically be used only because it contains the early promoter).

Most expression vectors are “shuttle” vectors, i.e., they are capable of replication in at least one class of organisms but can be transfected into another organism for expression. For example, a vector is cloned in E. coli and then the same vector is transfected into yeast or mammalian cells for expression even though it is not capable of replicating independently of the host cell chromosome.

DNA may also be amplified by insertion into the host genome. This is readily accomplished using Bacillus species as hosts, for example, by including in the vector a DNA sequence that is complementary to a sequence found in Bacillus genomic DNA. Transfection of Bacillus with this vector results in homologous recombination with the genome and insertion of heregulin DNA. However, the recovery of genomic DNA encoding heregulin is more complex than that of an exogenously replicated vector because restriction enzyme digestion is required to excise heregulin DNA. DNA can be amplified by PCR and directly transfected into the host cells without any replication component.

(iii) Selection Gene Component

Expression and cloning vectors should contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

One example of a selection scheme utilizes a drug to arrest growth of a host cell. Those cells that are successfully transformed with a heterologous gene express a protein conferring drug resistance and thus survive the selection regimen. Examples of such dominant selection use the drugs neomycin (Southern et al., J. Molec. Appl. Genet. 1: 327, 1982), mycophenolic acid (Mulligan et al., Science 209: 1422, 1980) or hygromycin (Sugden et al., Mol. Cell. Biol. 5: 410-413, 1985). The three examples given above employ bacterial genes under eukaryotic control to convey resistance to the appropriate drug G418 or neomycin (geneticin), xgpt (mycophenolic acid), or hygromycin, respectively.

Another example of suitable selectable markers for mammalian cells are those that enable the identification of cells competent to take up heregulin nucleic acid, such as dihydrofolate reductase (DHFR) or thymidine kinase. The mammalian cell transformants are placed under selection pressure which only the transformants are uniquely adapted to survive by virtue of having taken up the marker. Selection pressure is imposed by culturing the transformants under conditions in which the concentration of selection agent in the medium is successively changed, thereby leading to amplification of both the selection gene and the DNA that encodes heregulin. Amplification is the process by which genes in greater demand for the production of a protein critical for growth are reiterated in tandem within the chromosomes of successive generations of recombinant cells. Increased quantities of heregulin are synthesized from the amplified DNA.

For example, cells transformed with the DHFR selection gene are first identified by culturing all of the transformants in a culture medium that contains methotrexate (Mtx), a competitive antagonist of DHFR. An appropriate host cell when wild-type DHFR is employed is the Chinese hamster ovary (CHO) cell line deficient in DHFR activity, prepared and propagated as described by Urlaub and Chasin, Proc. Natl. Acad. Sci. USA, 77: 4216, 1980. The transformed cells are then exposed to increased levels of methotrexate. This leads to the synthesis of multiple copies of the DHFR gene, and, concomitantly, multiple copies of other DNA comprising the expression vectors, such as the DNA encoding heregulin. This amplification technique can be used with any otherwise suitable host, e.g., ATCC No. CCL61 CHO-K1, notwithstanding the presence of endogenous DHFR if, for example, a mutant DHFR gene that is highly resistant to Mtx is employed (EP 117,060). Alternatively, host cells (particularly wild-type hosts that contain endogenous DHFR) transformed or co-transformed with DNA sequences encoding heregulin, wild-type DHFR protein, and another selectable marker such as aminoglycoside 3′ phosphotransferase (APH) can be selected by cell growth in medium containing a selection agent for the selectable marker such as an aminoglycosidic antibiotic, e.g., kanamycin, neomycin, or G418 (see U.S. Pat. No. 4,965,199).

A suitable selection gene for use in yeast is the trp1 gene present in the yeast plasmid YRp7 (Stinchcomb et al., Nature, 282: 39, 1979; Kingsman et al., Gene, 7: 141, 1979; or Tschemper et al., Gene, 10: 157, 1980). The trp1 gene provides a selection marker for a mutant strain of yeast lacking the ability to grow in tryptophan, for example, ATCC No. 44076 or PEP4-1 (Jones, Genetics, 85: 12, 1977). The presence of the trp1 lesion in the yeast host cell genome then provides an effective environment for detecting transformation by growth in the absence of tryptophan. Similarly, Leu2-deficient yeast strains (ATCC 20,622 or 38,626) are complemented by known plasmids bearing the Leu2 gene.

(iv) Promoter Component

Expression and cloning vectors usually contain a promoter that is recognized by the host organism and is operably linked to heregulin nucleic acid. Promoters are untranslated sequences located upstream (5′) to the start codon of a structural gene (generally within about 100 to 1000 bp) that control the transcription and translation of a particular nucleic acid sequence, such as heregulin to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. At this time a large number of promoters recognized by a variety of potential host cells are well known. These promoters are operably linked to DNA encoding heregulin by removing the promoter from the source DNA by restriction enzyme digestion and inserting the isolated promoter sequence into the vector. Both the native heregulin promoter sequence and many heterologous promoters may be used to direct amplification and/or expression of heregulin DNA. However, heterologous promoters are preferred, as they generally permit greater transcription and higher yields of expressed heregulin as compared to the native heregulin promoter.

Promoters suitable for use with prokaryotic hosts include the b-lactamase and lactose promoter systems (Chang et al., Nature, 275: 615, 1978; and Goeddel et al., Nature 281: 544, 1979), alkaline phosphatase, a tryptophan (trp) promoter system (Goeddel, Nucleic Acids Res., 8: 4057, 1980 and EP 36,776), tPA (U.S. Pat. No. 5,641,655) and hybrid promoters such as the tac promoter (deBoer et al., Proc. Natl. Acad. Sci. USA 80: 21-25, 1983). However, other known bacterial promoters are suitable. Their nucleotide sequences have been published, thereby enabling a skilled worker operably to ligate them to DNA encoding heregulin (Siebenlist et al., Cell 20: 269, 1980) using linkers or adaptors to supply any required restriction sites. Promoters for use in bacterial systems also generally will contain a Shine-Dalgarno (S.D.) sequence operably linked to the DNA encoding heregulin.

Suitable promoting sequences for use with yeast hosts include the promoters for 3-phosphoglycerate kinase (Hitzeman et al., J. Biol. Chem., 255: 2073, 1980) or other glycolytic enzymes (Hess et al., J. Adv. Enzyme Reg 7: 149, 1968; and Holland, Biochemistry 17: 4900, 1978), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in Hitzeman et al., EP 73,657A. Yeast enhancers also are advantageously used with yeast promoters.

Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CXCAAT region where X may be any nucleotide. At the 3′ end of most eukaryotic genes is an AATAAA sequence that may be the signal for addition of the poly A tail to the 3′ end of the coding sequence. All of these sequences are suitably inserted into mammalian expression vectors.

Heregulin gene transcription from vectors in mammalian host cells may be controlled by promoters obtained from the genomes of viruses such as polyoma virus, fowlpox virus (UK 2,211,504, published Jul. 5, 1989), adenovirus (such as Adenovirus 2), bovine papilloma virus, avian sarcoma virus, cytomegalovirus, a retrovirus, hepatitis-B virus and most preferably Simian Virus 40 (SV40), from heterologous mammalian promoters, e.g., the actin promoter or an immunoglobulin promoter, from heat-shock promoters, and from the promoter normally associated with heregulin sequence, provided such promoters are compatible with the host cell systems.

The early and late promoters of the SV40 virus are conveniently obtained as an SV40 restriction fragment that also contains the SV40 viral origin of replication (Fiers et al., Nature, 273:113 (1978); Mulligan and Berg, Science, 209:1422-1427 (1980); Pavlakis et al., Proc. Natl. Acad. Sci. USA, 78: 7398-7402 (1981)). The immediate early promoter of the human cytomegalovirus is conveniently obtained as a HindIII E restriction fragment (Greenaway et al., Gene, 18: 355-360 (1982)). A system for expressing DNA in mammalian hosts using the bovine papilloma virus as a vector is disclosed in U.S. Pat. No. 4,419,446. A modification of this system is described in U.S. Pat. No. 4,601,978. See also Gray et al., Nature, 295: 503-508 (1982) on expressing cDNA encoding immune interferon in monkey cells; Reyes et al., Nature, 297: 598-601 (1982) on expression of human b-interferon cDNA in mouse cells under the control of a thymidine kinase promoter from herpes simplex virus; Canaani and Berg, Proc. Natl. Acad. Sci. USA, 79: 5166-5170 (1982) on expression of the human interferon b1 gene in cultured mouse and rabbit cells; and Gorman et al., Proc. Natl. Acad. Sci. USA, 79: 6777-6781 (1982) on expression of bacterial CAT sequences in CV-1 monkey kidney cells, chicken embryo fibroblasts, Chinese hamster ovary cells, HeLa cells, and mouse NIH-3T3 cells using the Rous sarcoma virus long terminal repeat as a promoter.

(v) Enhancer Element ComDonent

Transcription of a DNA encoding heregulin of this invention by higher eukaryotes is often increased by inserting an enhancer sequence into the vector. Enhancers are cis-acting elements of DNA, usually about from 10-300 bp, that act on a promoter to increase its transcription. Enhancers are relatively orientation and position independent having been found 5′ (Laimins et al., Proc. Nad. Acad. Sci. USA, 78: 993, 1981) and 3′ (Lusky et al., Mol. Cell Bio., 3: 1108, 1983) to the transcription unit, within an intron (Banerji et al., Cell, 33: 729, 1983) as well as within the coding sequence itself (Osborne et al., Mol. Cell Bio., 4: 1293, 1984). Many enhancer sequences are now known from mammalian genes (globin, elastase, albumin, a-fetoprotein and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers (see also Yaniv, Nature, 297: 17-18 (1982)) on enhancing elements for activation of eukaryotic promoters. The enhancer may be spliced into the vector at a position 5′ or 3′ to heregulin DNA, but is preferably located at a site 5′ from the promoter.

(vi) Transcription Termination Component

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′ untranslated regions of eukaryotic or viral DNAs or cDNAs. These regions contain nucleotide segments transcribed as polyadenylated fragments in the untranslated portion of the mRNA encoding heregulin. The 3′ untranslated regions also include transcription termination sites.

Construction of suitable vectors containing one or more of the above listed components the desired coding and control sequences employs standard ligation techniques. Isolated plasmids or DNA fragments are cleaved, tailored, and religated in the form desired to generate the plasmids required.

For analysis to confirm correct sequences in plasmids constructed, the ligation mixtures are used to transform E. coli K12 strain 294 (ATCC 31,446) and successful transformants selected by ampicillin or tetracycline resistance where appropriate. Plasmids from the transformants are prepared, analyzed by restriction endonuclease digestion, and/or sequenced by the method of Messing et al., Nucleic Acids Res. 9: 309 (1981) or by the method of Maxam et al., Methods in Enzymology 65: 499 (1980).

Particularly useful in the practice of this invention are expression vectors that provide for the transient expression in mammalian cells of DNA encoding heregulin. In general, transient expression involves the use of an expression vector that is able to replicate efficiently in a host cell, such that the host cell accumulates many copies of the expression vector and, in turn, synthesizes high levels of a desired polypeptide encoded by the expression vector. Transient expression systems, comprising a suitable expression vector and a host cell, allow for the convenient positive identification of polypeptides encoded by cloned DNAs, as well as for the rapid screening of such polypeptides for desired biological or physiological properties. Thus, transient expression systems are particularly useful in the invention for purposes of identifying analogs and variants of heregulin that have heregulin-like activity. Such a transient expression system is described in U.S. Pat. No. 5,024,939.

Other methods, vectors, and host cells suitable for adaptation to the synthesis of heregulin in recombinant vertebrate cell culture are described in Gething et al., Nature 293: 620-625, 1981; Mantei et al., Nature, 281: 40-46, 1979; Levinson et al., EP 117,060 and EP 117,058. A particularly useful expression plasmid for mammalian cell culture expression of heregulin is pRK5 (EP pub. no. 307,247) or pSVI6B (U.S. Ser. No. 07/441,574, filed Nov. 22, 1989, the disclosure of which is incorporated herein by reference).

D. Selection and Transformation of Host Cells

Suitable host cells for cloning or expressing the vectors herein are the prokaryote, yeast, or higher eukaryote cells described above. Suitable prokaryotes include eubacteria, such as Gram-negative or Gram-positive organisms, for example, E. coli, Bacilil such as B. subtilis, Pseudomonas species such as P. aeruginosa, Salmonella typhimudum, or Serratia marcescans. One preferred E. coli cloning host is E. coli 294 (ATCC 31,446), although other strains such as E. coli B, E. coli ₓ1776 (ATCC 31,537), and E. coli W3110 (ATCC 27,325) are suitable. These examples are illustrative rather than limiting. Preferably the host cell should secrete minimal amounts of proteolytic enzymes. Alternatively, in vitro methods of cloning, e.g., PCR or other nucleic acid polymerase reactions, are suitable.

In addition to prokaryotes, eukaryotic microbes such as filamentous fungi or yeast are suitable hosts for heregulin-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe (Beach and Nurse, Nature, 290: 140 (1981); EP 139,383, published May 2, 1985), Kluyveromyces hosts (U.S. Ser. No. 4,943,529) such as, e.g., K. lactis (Louvencourt et al., J. Bacteriol., 737 (1983); K. fragilis, K. bulgaticus, K. thernotolerans, and K. marxianus, yarrowia (EP 402,226); Pichia pastods (EP 183,070), Sreekrishna et al., J. Basic Microbiol., 28: 265-278 (1988); Candida, Trichodenna reesia (EP 244,234); Neurospora crassa (Case et al., Proc. Natl. Acad. Sci. USA, 76: 5259-5263 (1979), and filamentous fungi such as, e.g, Neurospora, Penicillium, Tolypocdadium (WO 91/00357, published Jan. 10, 1991), and Aspergillus hosts such as A. nidulans (Ballance et al., Biochem. Biophys. Res. Commun., 112: 284-289 (1983); Tilburn et al., Gene, 26: 205-221 (1983); Yelton et al., Proc. Natl. Acad. Sci. USA, 81:1470-1474 (1984) and A. niger (Kelly and Hynes, EMBO J., 4: 475-479 (1985)).

Suitable host cells for the expression of glycosylated heregulin polypeptide are derived from multicellular organisms. Such host cells are capable of complex processing and glycosylation activities. In principle, any higher eukaryotic cell culture is workable, whether from vertebrate or invertebrate culture. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori host cells have been identified (see, e.g., Luckow et al., Bio/Technology, 6: 47-55 (1988); Miller et al., in Genetic Engineering, Setlow, J. K. et al., eds., Vol. 8 (Plenum Publishing, 1986), pp. 277-279; and Maeda et al., Nature, 315: 592-594 (1985)). A variety of such viral strains are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can be utilized as hosts. Typically, plant cells are transfected by incubation with certain strains of the bacterium Agrobacterium tumefaciens, which has been previously manipulated to contain heregulin DNA. During incubation of the plant cell culture with A. tumefaciens, the DNA encoding heregulin is transferred to the plant cell host such that it is transfected, and will, under appropriate conditions, express heregulin DNA. In addition, regulatory and signal sequences compatible with plant cells are available, such as the nopaline synthase promoter and polyadenylation signal sequences (Depicker et al., J. Mol. Appl. Gen., 1: 561 (1982)). In addition, DNA segments isolated from the upstream region of the T-DNA 780 gene are capable of activating or increasing transcription levels of plant-expressible genes in recombinant DNA-containing plant tissue (see EP 321,196, published Jun. 21, 1989).

However, interest has been greatest in vertebrate cells, and propagation of vertebrate cells in culture (tissue culture) has become a routine procedure in recent years (Tissue Culture, Academic Press, Kruse and Patterson, editors (1973)). Examples of useful mammalian host cell lines are monkey kidney CV1 line transformed by SV40 (COS-7, ATCC CRL 1651); human embryonic kidney line (293 or 293 cells subcdoned for growth in suspension culture, Graham et al., J. Gen Virol., 36: 59, 1977); baby hamster kidney cells (BHK, ATCC CCL 10); Chinese hamster ovary cells-DHFR (CHO, Urlaub and Chasin, Proc. Natl. Acad. Sci. USA, 77: 4216 (1980)); mouse sertoli cells (TM4, Mather, Biol. Reprod., 23: 243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383: 44-68 (1982)); MRC 5 cells; FS4 cells; and a human hepatoma cell line (Hep G2). Preferred host cells are human embryonic kidney 293 and Chinese hamster ovary cells.

Host cells are transfected and preferably transformed with the above-described expression or cloning vectors of this invention and cultured in conventional nutrient media modified as appropriate for inducing promoters, selecting transformants, or amplifying the genes encoding the desired sequences.

Transfection refers to the taking up of an expression vector by a host cell whether or not any coding sequences are in fact expressed. Numerous methods of transfection are known to the ordinarily skilled artisan, for example, CaPO₄ and electroporation. Successful transfection is generally recognized when any indication of the operation of this vector occurs within the host cell.

Transformation means introducing DNA into an organism so that the DNA is replicable, either as an extrachromosomal element or by chromosomal integration. Depending on the host cell used, transformation is done using standard techniques appropriate to such cells. The calcium treatment employing calcium chloride, as described in section 1.82 of Sambrook et al., supra, is generally used for prokaryotes or other cells that contain substantial cell-wall barriers. Infection with Agrobacterium tumefaciens is used for transformation of certain plant cells, as described by Shaw et al., Gene, 23: 315 (1983) and WO 89/05859, published Jun. 29, 1989. For mammalian cells without such cell walls, the calcium phosphate precipitation method described in sections 16.30-16.37 of Sambrook et al, supra, is preferred. General aspects of mammalian cell host system transformations have been described by Axel in U.S. Pat. No. 4,399,216, issued Aug. 16, 1983. Transformations into yeast are typically carried out according to the method of Van Solingen et al., J. Bact., 130: 946 (1977) and Hsiao et al., Proc. Natl. Acad. Sci. (USA), 76: 3829 (1979). However, other methods for introducing DNA into cells such as by nuclear injection, electroporation, or protoplast fusion may also be used.

E. Culturing the Host Cells

Prokaryotic cells used to produce heregulin polypeptide of this invention are cultured in suitable media as described generally in Sambrook et al., supra.

The mammalian host cells used to produce heregulin of this invention may be cultured in a variety of media. Commercially available media such as Ham's F10 (Sigma), Minimal Essential Medium ((MEM), Sigma), RPMI-1640 (Sigma), and Dulbecco's Modified Eagle's Medium ((DMEM), Sigma) are suitable for culturing the host cells. In addition, any of the media described in Ham and Wallace, Meth. Enz., 58: 44 (1979), Barnes and Sato, Anal. Biochem., 102: 255 (1980), U.S. Pat. Nos. 4,767,704; 4,657,866; 4,927,762; or 4,560,655; WO 90/03430; WO 87/00195; U.S. Pat. Re. 30,985; U.S. Pat. No. 5,122,469 or U.S. Ser. No. 07/592,141, filed on Oct. 3, 1990, the disclosures of all of which are incorporated herein by reference, may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics (such as GENTAMYCIN drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

The host cells referred to in this disclosure encompass cells in in vifro culture as well as cells that are within a host animal.

It is further envisioned that heregulin of this invention may be produced by homologous recombination, or with recombinant production methods utilizing control elements introduced into cells already containing DNA encoding heregulin currently in use in the field. For example, a powerful promoter/enhancer element, a suppresser, or an exogenous transcription modulatory element is inserted in the genome of the intended host cell in proximity and orientation sufficient to influence the transcription of DNA encoding the desired heregulin. The control element does not encode heregulin of this invention, but the DNA is present in the host cell genome. One next screens for cells making heregulin of this invention, or increased or decreased levels of expression, as desired.

F. Detectinq Gene Amplification/Expression

Gene amplification and/or expression may be measured in a sample directly, for example, by conventional Southern blotting, Northern blotting to quantitate the transcription of mRNA (Thomas, Proc. Natl. Acad. Sci. USA, 77: 5201-5205 (1980)), dot blotting (DNA analysis), or in situ hybridization, using an appropriately labeled probe based on the sequences provided herein. Various labels may be employed, most commonly radioisotopes, particularly ³²P. However, other techniques may also be employed, such as using biotin-modified nucleotides for introduction into a polynucleotide. The biotin then serves as the site for binding to avidin or antibodies which may be labeled with a wide variety of labels, such as radionuclides, fluorescers, enzymes, or the like. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

Gene expression, alternatively, may be measured by immunological methods, such as immunohistochemical staining of tissue sections and assay of cell culture or body fluids, to quantitate directly the expression of gene product. With immunohistochemical staining techniques, a cell sample is prepared, typically by dehydration and fixation, followed by reaction with labeled antibodies specific for the gene product coupled where the labels are usually visually detectable such as enzymatic labels, fluorescent labels, luminescent labels, and the like. A particularly sensitive staining technique suitable for use in the present invention is described by Hsu et al., Am. J. Clin. Path., 75: 734-738 (1980).

Antibodies useful for immunohistochemical staining and/or assay of sample fluids may be either monoclonal or polyclonal, and may be prepared in any mammal. Conveniently, the antibodies may be prepared against a native heregulin polypeptide or against a synthetic peptide based on the DNA sequences provided herein as described further below.

G. Purification of The Heregulin Polypeptides

Heregulin is recovered from a cellular membrane fraction. Alternatively, a proteolytically cleaved or a truncated expressed soluble heregulin fragment or subdomain are recovered from the culture medium as a soluble polypeptide. A heregulin is recovered from host cell lysates when directly expressed without a secretory signal.

When heregulin is expressed in a recombinant cell other than one of human origin, heregulin is completely free of proteins or polypeptides of human origin. However, it is desirable to purify heregulin from recombinant cell proteins or polypeptides to obtain preparations that are substantially homogeneous as to heregulin. As a first step, the culture medium or lysate is centrifuged to remove particulate cell debris. The membrane and soluble protein fractions are then separated. Heregulin is then be purified from both the soluble protein fraction (requiring the presence of a protease) and from the membrane fraction of the culture lysate, depending on whether heregulin is membrane bound. The following procedures are exemplary of suitable purification procedures: fractionation on immunoaffinity or ion-exchange columns; ethanol precipitation; reverse phase HPLC; chromatography on silica, heparin SEPHAROSE or on a cation exchange resin such as DEAE; chromatofocusing; SDS-PAGE; ammonium sulfate precipitation; and gel filtration using, for example, SEPHADEX G-75.

Heregulin variants in which residues have been deleted, inserted or substituted are recovered in the same fashion as the native heregulin, taking account of any substantial changes in properties occasioned by the variation. For example, preparation of a heregulin fusion with another protein or polypeptide, e.g., a bacterial or viral antigen, facilitates purification; an immunoaffinity column containing antibody to the antigen can be used to adsorb the fusion. Immunoaffinity columns such as a rabbit polyclonal anti-heregulin column can be employed to absorb heregulin variant by binding it to at least one remaining immune epitope. A protease inhibitor such as phenylmethylsulfonylfluoride (PMSF) also may be useful to inhibit proteolytic degradation during purification, and antibiotics may be included to prevent the growth of adventitious contaminants. One skilled in the art will appreciate that purification methods suitable for native heregulin may require modification to account for changes in the character of heregulin variants or upon expression in recombinant cell culture.

H. Covalent Modifications of Heregulin

Covalent modifications of heregulin polypeptides are included within the scope of this invention. Both native heregulin and amino acid sequence variants of heregulin optionally are covalently modified. One type of covalent modification included within the scope of this invention is a heregulin polypeptide fragment. Heregulin fragments, such as HRG-GDF, having up to about 40 amino acid residues are conveniently prepared by chemical synthesis, or by enzymatic or chemical cleavage of the full-length heregulin polypeptide or heregulin variant polypeptide. Other types of covalent modifications of heregulin or fragments thereof are introduced into the molecule by reacting targeted amino acid residues of heregulin or fragments thereof with an organic derivatizing agent that is capable of reacting with selected side chains or the N- or C-terminal residues.

Cysteinyl residues most commonly are reacted with α-haloacetates (and corresponding amines), such as chloroacetic acid or chioroacetamide, to give carboxymethyl or carboxyamidomethyl derivatives. Cysteinyl residues also are derivatized by reaction with bromotrifluoroacetone, α-bromo-β-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylpyrocarbonate at pH 5.5-7.0 because this agent is relatively specific for the histidyl side chain. Para-bromophenacyl bromide also is useful; the reaction is preferably performed in 0.1M sodium cacodylate at pH 6.0.

Lysinyl and amino terminal residues are reacted with succinic or other carboxylic acid anhydrides. Derivatization with these agents has the effect of reversing the charge of the lysinyl residues. Other suitable reagents for derivatizing a-amino-containing residues include imidoesters such as methyl picolinimidate; pyridoxal phosphate; pyridoxal; chloroborohydride; trinitrobenzenesulfonic acid; O-methylisourea; 2,4-pentanedione; and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKₐ of the guanidine functional group. Furthermore, these reagents may react with the groups of lysine as well as the arginine epsilon-amino group.

The specific modification of tyrosyl residues may be made, with particular interest in introducing spectral labels into tyrosyl residues by reaction with aromatic diazonium compounds or tetranitromethane. Most commonly, N-acetylimidizole and tetranitromethane are used to form O-acetyl tyrosyl species and 3-nitro derivatives, respectively. Tyrosyl residues are iodinated using ¹²⁵I or ¹³¹I to prepare labeled proteins for use in radioimmunoassay, the chloramine T method described above being suitable.

Carboxyl side groups (aspartyl or glutamyl) are selectively modified by reaction with carbodlimides (R′—N═C═N—R′), where R and R′ are different alkyl groups, such as 1-cyclohexyl-3-(2-morpholinyl-4-ethyl)carbodiimide or 1-ethyl-3-(4-azonia-4,4-dimethylpentyl)carbodiimide. Furthermore, aspartyl and glutamyl residues are converted to asparaginyl and glutaminyl residues by reaction with ammonium ions.

Derivatization with bifunctional agents is useful for crosslinking heregulin to a water-insoluble support matrix or surface for use in a method for purifying anti-heregulin antibodies, and vice versa, Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-((p-azidophenyl)dithio)propioimida yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are employed for protein immobilization.

Glutaminyl and asparaginyl residues are frequently deamidated to the corresponding glutamyl and aspartyl residues, respectively. Alternatively, these residues are deamidated under mildly acidic conditions. Either form of these residues falls within the scope of this invention.

Other modifications include hydroxylation of proline and lysine, phosphorylation of hydroxyl groups of seryl or threonyl residues, methylation of the a-amino groups of lysine, arginine, and histidine side chains (T. E. Creighton, Proteins: Structure and Molecular Properties, W. H. Freeman & Co., San Francisco, pp. 79-86 (1983)), acetylation of the N-terminal amine, and amidation of any C-terminal carboxyl group.

Heregulin optionally is fused with a polypeptide heterologous to heregulin. The heterologous polypeptide optionally is an anchor sequence such as that found in a phage coat protein such as M13 gene III or gene VIII proteins. These heterologous polypeptides can be covalently coupled to heregulin polypeptide through side chains or through the terminal residues.

Heregulin may also be covalently modified by altering its native glycosylation pattern. One or more carbohydrate substitutents in these embodiments, are modified by adding, removing or varying the monosaccharide components at a given site, or by modifying residues in heregulin as that glycosylation sites are added or deleted.

Glycosylation of polypeptides is typically either N-linked or O-linked. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in a polypeptide creates a potential glycosylation site. O-linked glycosylation refers to the attachment of one of the sugars N-acetylgalactosamine, galactose, or xylose, to a hydroxyamino acid, most commonly serine or threonine, although 5-hydroxyproline or 5-hydroxylysine may also be used.

Glycosylation sites are added to heregulin by altering its amino acid sequence to contain one or more of the above-described tri-peptide sequences (for N-linked glycosylation sites). The alteration may also be made by the addition of, or substitution by, one or more serine or threonine residues to heregulin (for O-linked glycosylation sites). For ease, heregulin is preferably altered through changes at the DNA level, particularly by mutating the DNA encoding heregulin at preselected bases such that codons are generated that will translate into the desired amino acids.

Chemical or enzymatic coupling of glycosides to heregulin increases the number of carbohydrate substituents. These procedures are advantageous in that they do not require production of the polypeptide in a host cell that is capable of N- and O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO 87/05330, published Sep. 11, 1987, and in Aplin and Wriston (CRC Crit. Rev. Biochem., pp. 259-306 (1981)).

Carbohydrate moieties present on an heregulin also are removed chemically or enzymatically. Chemical deglycosylation requires exposure of the polypeptide to the compound trifluoromethanesulfonic acid, or an equivalent compound. This treatment results in the cleavage of most or all sugars except the linking sugar (N-acetylglucosamine or N-acetylgalactosamine), while leaving the polypeptide intact. Chemical deglycosylation is described by Hakimuddin et al. (Arch. Biochem. Biophys., 259:52 (1987)) and by Edge et al. (Anal. Biochem., 118:131 (1981)). Carbohydrate moieties are removed from heregulin by a variety of endo- and exo-glycosidases as described by Thotakura et al. (Meth. Enzymol., 138:350 (1987)).

Glycosylation also is suppressed by tunicamycin as described by Duskin et al. (J. Biol. Chem., 257:3105 (1982)). Tunicamycin blocks the formation of protein-N-glycoside linkages.

Heregulin may also be modified by linking heregulin to various nonproteinaceous polymers, e.g., polyethylene glycol, polypropylene glycol or polyoxyalkylenes, in the manner set forth in U.S. Pat. Nos. 4,640,835; 4,496,689; 4,301,144; 4,670,417; 4,791,192 or 4,179,337.

One preferred way to increase the in vivo circulating half life of non-membrane bound heregulin is to conjugate it to a polymer that confers extended half-life, such as polyethylene glycol (PEG). (Maxfield, et al, Polymer 16,505-509 (1975); Bailey, F. E., et al, in Nonionic Surfactants (Schick, M. J., ed.) pp.794-821, 1967); (Abuchowski, A. et al., J. Biol. Chem. 252, 3582-3586, 1977; Abuchowski, A. et al., Cancer Biochem. Biophys. 7, 175-186, 1984); (Katre, N. V. et al., Proc. Natl. Acad. Sci., 84, 1487-1491, 1987; Goodson, R. et al. Bio Technology, 8, 343-346, 1990). Conjugation to PEG also has been reported to have reduced immunogenicity and toxicity (Abuchowski, A. et al., J. Biol. Chem., 252, 3578-3581, 1977).

Heregulin may also be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Osol, A., Ed., (1980).

Those skilled in the art will be capable of screening variants in order to select the optimal variant for the purpose intended. For example, a change in the immunological character of heregulin, such as a change in affinity for a given antigen or for the HER2 receptor, is measured by a competitive-type immunoassay using a standard or control such as a native heregulin (in particular native heregulin-GFD). Other potential modifications of protein or polypeptide properties such as redox or thermal stability, hydrophobicity, susceptibility to proteolytic degradation, stability in recombinant cell culture or in plasma, or the tendency to aggregate with carriers or into multimers are assayed by methods well known in the art.

I. Herepulin Antibody Preparation

The antibodies of this invention are obtained by routine screening and include polyclonal antibodies, monoclonal antibodies and fragments thereof.

Polyclonal antibodies are preferably raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

Animals are immunized against the antigen, immunogenic conjugates, or derivatives by combining, e.g., 100 ug or 5 ug of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

Monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster or macaque monkey, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp.59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOP-21 and M.C.-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

The binding affinity of the monoclonal antibody can, for example, be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp.59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-SEPHAROSE, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. Coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant production of antibodies will be described in more detail below.

Hybridoma cell lines producing antibodies are identified by screening the culture supernatants for antibody which binds to HER2 and/or HER3 receptors. This is routinely accomplished by conventional immunoassays using soluble receptor preparations or by FACS using celi-bound receptor and labeled candidate antibody. Agonist antibodies are preferably antibodies which stimulate autophosphorylation in the heregulin tyrosine autophosphorylation assay described above.

The hybrid cell lines can be maintained in culture in vitro in cell culture media. The cell lines of this invention can be selected and/or maintained in a composition comprising the continuous cell line in hypoxanthine-aminopterin thymidine (HAT) medium. In fact, once the hybridoma cell line is established, it can be maintained on a variety of nutritionally adequate media. Moreover, the hybrid cell lines can be stored and preserved in any number of conventional ways, including freezing and storage under liquid nitrogen. Frozen cell lines can be revived and cultured indefinitely with resumed synthesis and secretion of monoclonal antibody. The secreted antibody is recovered from tissue culture supernatant by conventional methods such as precipitation, ion exchange chromatography, affinity chromatography, or the like. The antibodies described herein are also recovered from hybridoma cell cultures by conventional methods for purification of IgG or IgM as the case may be that heretofore have been used to purify these immunoglobulins from pooled plasma, e.g., ethanol or polyethylene glycol precipitation procedures.

Human antibodies may be used and are preferable. Such antibodies can be obtained by using human hybridomas (Cote et al., Monoctonal Antibodies and Cancer Therapy, Alan R. Liss, p. 77 (1985)). Chimeric antibodies, Cabilly et al., U.S. Pat. No. 4,816,567, (Morrison et al., Proc. Natl. Acad. Sci., 81:6851 (1984); Neuberger et al., Nature 312:604 (1984); Takeda et al., Nature 314:452 (1985)) containing a murine anti-HER2/HER3 variable region and a human constant region of appropriate biological activity (such as ability to activate human complement and mediate ADCC) are within the scope of this invention, as are humanized antibodies produced by conventional CDR-grafting methods(Riechmann et al., Nature 332:333-327(1988); EP 0328404 A1; EP 02394000 A2).

Techniques for creating recombinant DNA versions of the antigen-binding regions of antibody molecules (Fab or variable regions fragments) which bypass the generation of monoclonal antibodies are also encompassed within the practice of this invention. One extracts antibody-specific messenger RNA molecules from immune system cells taken from an immunized subject, transcribes these into complementary DNA (cDNA), and clones the cDNA into a bacterial expression system and selects for the desired binding characteristic. The Scripps/Stratagene method uses a bacteriophage lambda vector system containing a leader sequence that causes the expressed Fab protein to migrate to the periplasmic space (between the bacterial cell membrane and the cell wall) or to be secreted. One can rapidly generate and screen great numbers of functional Fab fragments to identify those which bind the receptors with the desired characteristics. Alternatively, the antibodies can be prepared by the phage display techniques described in Hoogenboom, Tibtech February 1997 (vol 15); Neri et al., Cell Biophysics 27:47-61 (1995); Winter et al., Annu. Rev. Immunol. 12:433-55 (1994); and Soderlind et al., Immunol. Rev. 130:109-124 (1992) and the references described therein as well as the monovalent phage display technique described in Lowman et al., Biochem. 30:10832-10838 (1991).

2. Therapeutic Compositions, Administration and Use of Heregulins and Agonist Antibodies

The heregulin are used in the present invention to induce inner-ear-supporting cell proliferation to enhance new hair cell generation. These effects allow treatment of disease states associated with tissue damage, for example, ototoxic injury, or acoustic assault, degenerative hearing loss or balance impairments, or damage associated with surgery or physical injury.

The field of cochlear implantation has also provided insights into both the short- and long-term effects of cochlear fenestration on inner ear function. Administration of growth factors to the inner ears of animals is now possible with the use of implanted catheters and miniature infusion pumps. Localized application of heregulin to the human inner ear can be performed to treat inner ear disorders related to hair cell disfunction.

Therapeutic formulations of heregulin or agonist antibody are prepared for storage by mixing the heregulin protein having the desired degree of purity with optional physiologically acceptable carriers, excipients, or stabilizers (Remington's Pharmaceutical Sciences, supra), in the form of lyophilized cake or aqueous solutions. Acceptable carriers, excipients or stabilizers are nontoxic to recipients at the dosages and concentrations employed, and include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN, PLURONICS or polyethylene glycol (PEG).

Heregulin or agonist antibody to be used for in vivo administration must be sterile. This is readily accomplished by filtration through sterile filtration membranes, prior to or following lyophilization and reconstitution. The heregulin or antibody ordinarily will be stored in lyophilized form or in solution.

Therapeutic heregulin or antibody compositions generally are placed into a container having a sterile access port, for example, an intravenous solution bag or vial having a stopper pierceable by a hypodermic injection needle.

The route of heregulin or antibody administration is in accord with known methods, e.g., injection or infusion administration to the inner ear, or intralesional routes, or by sustained release systems as noted below. The heregulin ligand may be administered continuously by infusion or by bolus injection. An agonist antibody is preferably administered in the same fashion.

The heregulin, heregulin variant or fragment and agonist antibodies may be spray dried or spray freeze dried using known techniques (Yeo et al, Biotech. and Bioeng., 41:341-346 (1993); Gombotz et al, PCT/US90/02421).

Suitable examples of sustained-release preparations include semipermeable matrices of solid hydrophobic polymers containing the protein, which matrices are in the form of shaped articles, e.g. films, or microcapsules. Examples of sustained-release matrices include polyesters, hydrogels (e.g., poly(2-hydroxyethyl-methacrylate) as described by Langer et al., J. Biomed. Mater. Res., 15: 167-277 (1981) and Langer, Chem. Tech., 12: 98-105 (1982) or poly(vinylalcohol)), polylactides (U.S. Pat. No. 3,773,919, EP 58,481), copolymers of L-glutamic acid and gamma ethyl-L-glutamate (Sidman et al., Biopolymers, 22: 547-556 (1983)), non-degradable ethylene-vinyl acetate (Langer et al., supra), degradable lactic acid-glycolic acid copolymers such as the LUPRON DEPOT (injectable microspheres composed of lactic acid-glycolic acid copolymer and leuprolide acetate), and poly-D-(−)-3-hydroxybutyric acid (EP 133,988). While polymers such as ethylene-vinyl acetate and lactic acid-glycolic acid enable release of molecules for over 100 days, certain hydrogels release proteins for shorter time periods. When encapsulated proteins remain in the body for a long time, they may denature or aggregate as a result of exposure to moisture at 37° C., resulting in a loss of biological activity and possible changes in immunogenicity. Rational strategies can be devised for protein stabilization depending on the mechanism involved. For example, if the aggregation mechanism is discovered to be intermolecular S-S bond formation through thio-disulfide interchange, stabilization may be achieved by modifying sulfhydryl residues, lyophilizing from acidic solutions, controlling moisture content, using appropriate additives, and developing specific polymer matrix compositions.

Sustained-release heregulin or antibody compositions also include liposomally entrapped heregulin or antibody. Liposomes containing heregulin or antibody are prepared by methods known per se: DE 3,218,121; Epstein et al., Proc. Nat. Acad. Sci. USA, 82: 3688-3692 (1985); Hwang et al., Proc. Natl. Acad. Sci. USA, 77: 4030-4034 (1980); EP 52,322; EP 36,676; EP 88,046; EP 143,949; EP 142,641; Japanese patent application 83-118008; U.S. Pat. Nos. 4,485,045 and 4,544,545; and EP 102,324. Ordinarily the liposomes are of the small (about 200-800 Angstroms) unilamelar type in which the lipid content is greater than about 30 mol. % cholesterol, the selected proportion being adjusted for the optimal heregulin therapy. Liposomes with enhanced circulation time are disclosed in U.S. Pat. No. 5,013,556.

Accordingly, the methods and compositions of the invention find use for the prevention and treatment of opportunistic infections in animals and man which are immunosuppressed as a result of either congenital or acquired immunodeficiency or as a side-effect of chemotherapeutic treatment. According in an alternate embodiment of the present invention, a composition of the invention is used advantageously in combination with a known antimicrobial agent to provide improved methods and compositions to prevent and/or treat diseases induced by gram positive bacteria including, but not limited to: Staphylococcus aureus, Streptococcus pneumonia, Hemophilus influenza; gram negative bacteria including, but not limited to: Escherichia coli; Bacterium enteritis, Francisella tularensis; acid-fast bacteria including, but not limited to Mycobacterium tuberculosis, and Mycobacterium leprae. Use of a combination of an antimicrobial agent together with a composition of the invention is advantageous with antibacterial aminoglycosides such as gentamicin, streptomycin, and the like which are known to have serious ototoxicity, which reduce the usefulness of such antimicrobial agents. Use of a composition of the invention in combination with such agents permits a lower dosage of the toxic antimicrobial agents while still achieving therapeutic (antibacterial) effectiveness.

In some embodiments the composition of the invention is co-administered with an ototoxin. For example, an improved method is provided for treatment of infection of a mammal by administration of an aminoglycoside antibiotic, the improvement comprising administering a therapeutically effective amount of heregulin or agonist, to the patient in need of such treatment to reduce or prevent ototoxin-induced hearing impairment associated with the antibiotic. In yet another embodiment is provided an improved method for treatment of cancer in a mammal by administration of a chemotherapeutic compound, the improvement comprises administering a therapeutically effective amount of a composition of the invention to the patient in need of such treatment to reduce or prevent ototoxin-induced hearing impairment associated with the chemotherapeutic drug.

Also provided herein are methods for promoting new inner ear hair cells by inducing inner ear supporting cell proliferation, regeneration, or growth upon, prior to, or after exposure to an agent or effect that is capable of inducing a hearing or balance impairment or disorder. Such agents and effects are those described herein. The method includes the step of administering to the inner ear hair cell an effective amount of heregulin or agonist or factor disclosed herein as useful. Preferably, the method is used upon, prior to, or after exposure to a hearing-impairing ototoxin.

In one embodiment the methods of treatment are applied to hearing impairments resulting from the administration of a chemotherapeutic agent to treat its ototoxic side-effect. Ototoxic chemotherapeutic agents amenable to the methods of the invention include, but are not limited to an antineoplastic agent, including cisplatin or cisplatin-like compounds, taxol or taxol-like compounds, and other chemotherapeutic agents believed to cause ototoxin-induced hearing impairments, e.g., vincristine, an antineoplastic drug used to treat hematological malignancies and sarcomas.

In one embodiment the methods of the invention are applied to hearing impairments resulting from the administration of quinine and its synthetic substitutes, typically used in the treatment of malaria, to treat its ototoxic side-effect.

In another embodiment the methods of the invention are applied to hearing impairments resulting from administration of a diuretic to treat its ototoxic side-effect. Diuretics, particularly “loop” diuretics, i.e. those that act primarily in the Loop of Henle, are candidate ototoxins. Illustrative examples, not limiting to the invention method, include furosemide, ethacrynic acid, and mercurials. Diuretics are typically used to prevent or eliminate edema. Diuretics are also used in nonedematous states such as hypertension, hypercalcemia, idiopathic hypercalciuria, and nephrogenic diabetes insipidus.

In another embodiment the compositions of the invention are administered with an agent that promotes neuronal cell growth, proliferation, or regeneration. As known in the art, low concentrations of gentamicin preferentially kills hair cells while the damage to the ganglion neurons is not significant. However, high concentrations of gentamicin induce degeneration of ganglion neurons as well as hair cells. Accordingly, this dual toxicity of aminoglycosides can be treated by the methods of the invention, preferably with compositions of the invention.

The heregulin or agonist is directly administered to the patient by any suitable technique, including parenterally, intranasally, intrapulmonary, orally, or by absorption through the skin. If they are administered together, they need not be administered by the same route. They can be administered locally or systemically. Examples of parenteral administration include subcutaneous, intramuscular, intravenous, intraarterial, and intraperitoneal, and intracochlear administration. They can be administered by daily subcutaneous injection. They can be administered by implants. They can be administered in liquid drops to the ear canal, delivered to the scala tympani chamber of the inner ear, or provided as a diffusible member of a cochlear hearing implant.

The heregulin or antibody agonist, can be combined and directly administered to the mammal by any suitable technique, including infusion and injection. The specific route of administration will depend, e.g., on the medical history of the patient, including any perceived or anticipated side effects using heregulin alone, and the particular disorder to be corrected. Examples of parenteral administration include subcutaneous, intramuscular, intravenous, intraarterial, and intraperitoneal administration. Most preferably, the administration is by continuous infusion (using, e.g., slow-release devices or minipumps such as osmotic pumps or skin patches), or by injection (using, e.g., intravenous or subcutaneous means). The administration may also be as a single bolus or by slow-release depot formulation. The agonist(s) is administered in an acute or chronic fashion, as may be required, for prophylactic and therapeutic applications, by a number of routes including: injection or infusion by intravenous, intraperitoneal, intracerebral, intramuscular, intradermally, intraocular, intraarterial, subcutaneously, or intralesional routes, topical administration, orally if an orally active small molecule is employed, using sustained-release systems as noted below, or by an indwelling catheter using a continuous administration means such as a pump, by patch, or implant systems, e.g., implantation of a sustained-release vehicle or immuno-isolated cells secreting the growth factor(s) and/or neurotrophin(s). Agonist(s) is administered continuously by infusion or by periodic bolus injection if the clearance rate is sufficiently slow, or by administration into the blood stream, lymph, CNS or spinal fluid. A preferred administration mode is directly to the affected portion of the ear or vestibule, topically as by implant for example, and, preferably to the affected hair cells, their supporting cells, and (optionally to) associated neurons, so as to direct the molecule to the source and minimize side effects of the agonists.

As noted the compositions can be injected through chronically implanted cannulas or chronically infused with the help of osmotic minipumps. Subcutaneous pumps are available that deliver proteins through a small tubing to the appropriate area. Highly sophisticated pumps can be refilled through the skin and their delivery rate can be set without surgical intervention. Examples of suitable administration protocols and delivery systems involving a subcutaneous pump device or continuous infusion through a totally implanted drug delivery system are those used for the administration of dopamine, dopamine agonists, and cholinergic agonists to Alzheimer patients and animal models for Parkinson's disease described by Harbaugh, J. Neural Transm. Suppl., 24: 271-277 (1987) and DeYebenes et al., Mov. Disord., 2: 143-158 (1987), the disclosures of which are incorporated herein by reference. It is envisioned that it may be possible to introduce cells actively producing agonist into areas in need of increased concentrations of agonist.

An effective amount of heregulin or antibody to be employed therapeutically will depend, for example, upon the therapeutic objectives, the route of administration, and the condition of the patient. Also, the amount of heregulin polypeptide will generally be less than the amount of an agonist antibody. Accordingly, it will be necessary for the therapist to titer the dosage and modify the route of administration as required to obtain the optimal therapeutic effect. A typical daily dosage might range from about 1 μg/kg to about 1 mg/kg and up to 100 mg/kg or more, depending on the factors mentioned above. Typically, the clinician will administer heregulin or antibody until a dosage is reached that achieves the desired effect. The progress of this therapy is easily monitored by conventional diagnostic clinical tests for hearing or balance improvement.

In a further embodiment, inner-ear-supporting cells may be obtained or isolated from a mammalian tissue to obtain a normal inner-ear-supporting cell sample using techniques well known in the art (biopsy, etc.). This sample may then be treated with a heregulin protein in order to induce hair cell or inner-ear-supporting cell growth and/or proliferation in the sample thereby expanding the population of inner-ear-supporting cells. Typically, heregulin will be added to the in vitro inner-ear-supporting cell culture at a concentration of about 0.1 to about 100 nM preferably 1-50 nM. If desired, the primary inner-ear-supporting cells may be cultured in vitro for several generations in order to sufficiently expand the hair cell or inner-ear-supporting cell population. The hair cell or inner-ear-supporting cells are cultured under conditions suitable for mammalian cell culture as discussed above. After expansion, the expanded sample is reintroduced into the mammal for the purpose of re-epithelializing the mammalian tissue.

The methods and procedures described herein with respect to HRG-α or heregulin in general may be applied similarly to other heregulin such as HRG-β1, HRG-β2 and HRG-β3 and to variants thereof, as well as to the antibodies. All references cited in this specification are expressly incorporated by reference. The following examples are offered by way of illustration and not by way of limitation.

### EXAMPLES

### Example I

Characterization of Inner-Ear-Supporting Cell Cultures

Cultured undissociated utricular epithelial cells were determined to express features of epithelial cells, but not those of fibroblast, glial or neuronal cells. Utricular sheets were prepared by the method of Zheng et al. (Journal of Neuroscience, 17(21):8270-82 (1997)) and Zheng et al. (Journal of Neuroscience, 17(1):216-226 (1997)). Utricular sheet cultures contained primarily supporting cells and hair cells.

Utricular epithelial sheets were separated from either postnatal day 3 (P3) or day 4-5 Wistar rats using 0.5 mg/ml thermolysin (Sigma; in Hank's calcium and magnesium-free balanced salt solution) for 30 min at 37° C., based on the method reported previously (Corwin et al., 1995). The epithelial sheets were then incubated in a mixture of 0.125% trypsin and 0.125% collagenase for 8 min at 37° C. The enzyme activity was inactivated with a mixture of 0.005% soybean trypsin inhibitor (Sigma) and 0.005% DNase (Worthington) before being pipetted up and down with a 1 ml pipette tip 10 times in 0.05% DNase in BME. A 5% fetal bovine serum-supplemented medium was used. The cell suspension was finally plated in polylysine (500 μg/mI) coated 96-well plate (for tritiated thymidine assays) or 16-well LabTek slides (for BrdU labeling and other immunocytochemistry) in 200 μl of serum-containing medium (DMEM plus 5% fetal bovine serum, 4.5 mg/ml glucose, 2 mM glutamine, 25 ng/ml fungizone and 10 units/ml penicillin) at a density of approximately 70 cells/mm². Typically, cells prepared from 4 litters of pups (40 P3 or P4-5 rats) were equally aliquoted into 80 wells.

While most isolated single cells died after 2 days in culture, the cell clumps containing approximately 10-80 cells survived well and grew in patches in the serum-supplemented medium. Immunocytochemical staining with different types of cell markers revealed that these cultured cells expressed epithelial cell antigens including a tight junction protein ZO1, F-actin, and cytokeratin. They did not express antigens for other types of cells, such as glial filament protein (GFAP), the oligodendrocyte antigen (myelin), neurofilament protein or fibroblast antigens, vimentin, and Thy1.1. Because the utricular epithelial sheets contained mainly supporting cells and hair cells, the vast majority of the surviving, mitotic cells in the cultures represented a population of utricular supporting cells.

### Example II

Stimulation of Supporting Cell Proliferation and Hair Cell Generation

To examine whether any of the presently known growth factors stimulate proliferation of the utricular supporting cells and stimulates an increase in number of dividing cells, bromo-deoxyuridine (BrdU) immunocytochemistry was performed as described in Zheng et al. (Journal of Neuroscience, 17(1):216-226 (1997)). Briefly, after 1 day in culture, BrdU (1:400; Amersham cell proliferation kit) was added to the culture medium for 24 h. The cultures were fixed in 4% paraformaldehyde (30 min), treated with 2 N HCl (40 min), and incubated with an anti-BrdU monoclonal antibody (Becton-Dickinson, 1:40 in phosphate buffered saline containing 0.1% Triton-X100) overnight at 4° C. The cultures were then processed with a Vector ABC kit. After diaminobenzidine-peroxidase reaction, the cells were dehydrated with ethanol, cleared in Histoclear (American Histology) and mounted in Permount (Fisher). BrdU-positive cells were counted from the entire areas of 10 or more culture wells for each of the experimental groups. Data was expressed as mean±s.e.m. Two-tailed, unpaired t-test was used for statistical analysis.

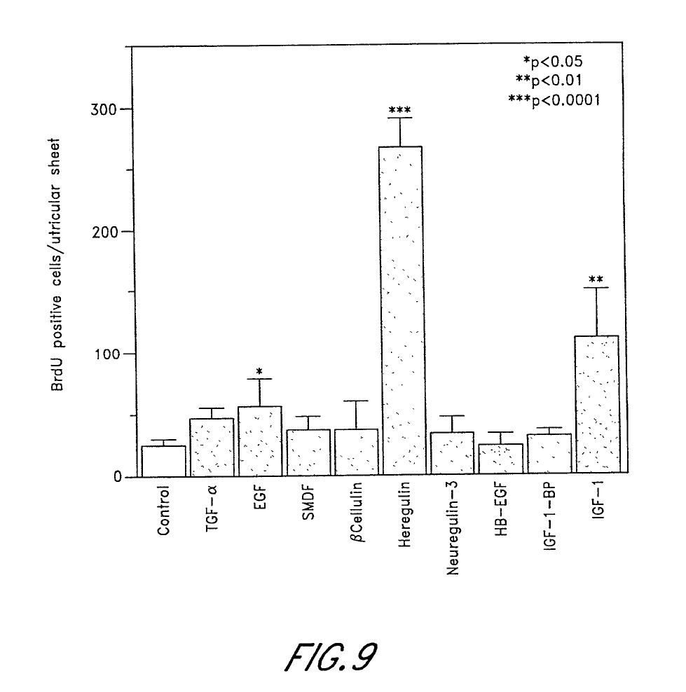

A much greater number of BrdU-positive cells were seen in the cultures containing heregulin (HRG-β1-177-244) than any of the other factors known to activate Her receptors. Cell counts performed from the control cultures and cultures containing confirmed that heregulin significantly enhanced proliferation of the utricular supporting cells (p<0.0001, FIG. 9). IGF-1 at 100 nM, TGF-α at 100 nM (R & D Systems), EGF 100 nM (Collaborative Research), SMDF at 100 nM, βcellulin at 100 nM, neuregulin3 at 20 nM, HB-EGF at 100 nM (Elenius et al., EMBO Journal 16(6):1268-78 (1997)), and IGF1-binding protein at 100 nM were weaker mitogens, if at all, compared to heregulin. SMDF polypeptides are prepared as described in WO 96/15244. Neuregulin-3, a neural tissue-enriched protein that binds and activates erbB4, was prepared as described in Zhang et al., Proceedings of the National Academy of Sciences, 94(18):9562-7 (1997). β-cellulin was prepared as described in Daly et al., Cancer Research. 57(17):3804-11 (1997). The EGF-like domain of HRGβ1₍₁₇₇₋₂₄₄₎ was expressed in E. coli, purified and radioiodinated as described previously (Sliwkowski et al. J. Biol. Chem. 269:14661-14665 (1994)). All factors were added to the cultures at the time when the cells were plated.

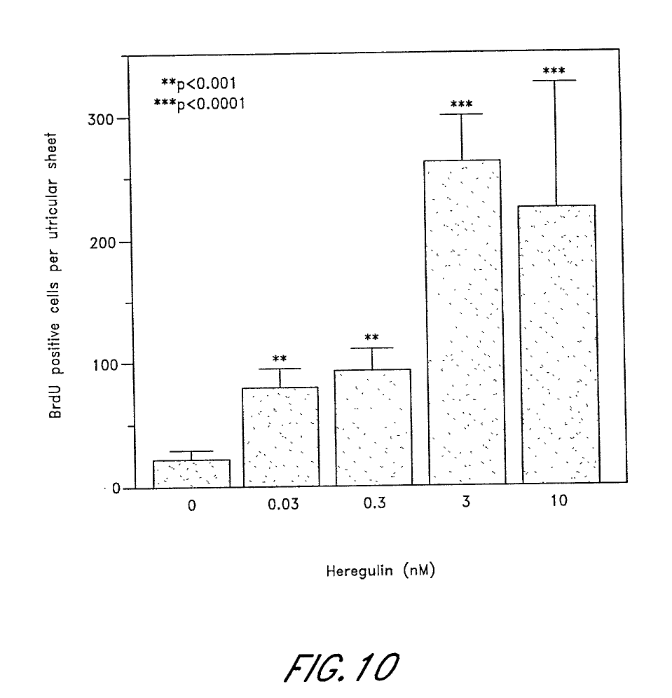

To determine whether the effect of heregulin was dose-dependent, a dose-dependent study was carried out in the utricular epithelial sheet cultures at a range of 0.03 nM to 10 nM heregulin (FIG. 10). A heregulin-dose-dependent increase in the number of BrdU positive cells was observed. Maximal effect of heregulin was seen at 3 nM.

### Example III

Stimulation of Supportinq Cell Proliferation and Hair Cell Generation in Utricle Explant Cultures

An organotype utricle explant culture that utilizes a 3-D collagen matrix cultures and maintains its normal, in vivo architecture was prepared as described in Zheng et al. (Journal of Neuroscience, 17(21):8270-82 (1997)). This system provides an excellent means to test the effect of heregulin on supporting cell proliferation in a physiologically significant system that mimics the in vivo state. In particular, the effects of heregulin after ototoxic-induced damage (e.g. antibiotic gentamycin) were examined.

The utricle was dissected with the vestibular ganglion attached, from P3 Wistar rats and cultured in three-dimensional (“3-D”) collagen gel in serum free medium. In one embodiment a droplet (20 μl) of freshly made collagen gel which was placed on the bottom of a 35 mm Nunc tissue culture dish, modified from what described previously (Gao et al., Neuron 6:705-715 (1991)) as follows. Rat tail collagen (type I, Collaborative Research) was mixed with 10× BME medium and 2% sodium carbonate in a ratio of 10:1:1 and placed on ice just before use. The collagen matrix containing the utricle explant was incubated at 37° C. for 5-10 min until gellation. The matrix was then cultured in defined serum-free medium using sufficient medium to cover the explant (2 ml of serum-free medium (BME plus serum-free supplement (Sigma I-1884), 1% BSA, 2 mM glutamine, and 5 mg/ml glucose; containing no antibiotics). The culture medium was changed every other day thereafter.

Embedding the utricle explants in the 3-D collagen was better for maintaining their normal architecture than floating the explants or placing the explants on a monolayer substrate, since the explant tissue could be kept unfolded and cell migration out of the tissue could be limited. Utricle explant whole mount cultures maintained normal architecture in the 3-D collagen gel cultures as observed by Nomarski micrographs of utricle tissue dissected from P3 rats and grown for 2 days in vitro at low and high magnifications. The hair cells in the explants grew well and maintained their normal connectivity. The hair cells remained in their normal locations. No gross cell death of hair cells occurred under this culture condition.

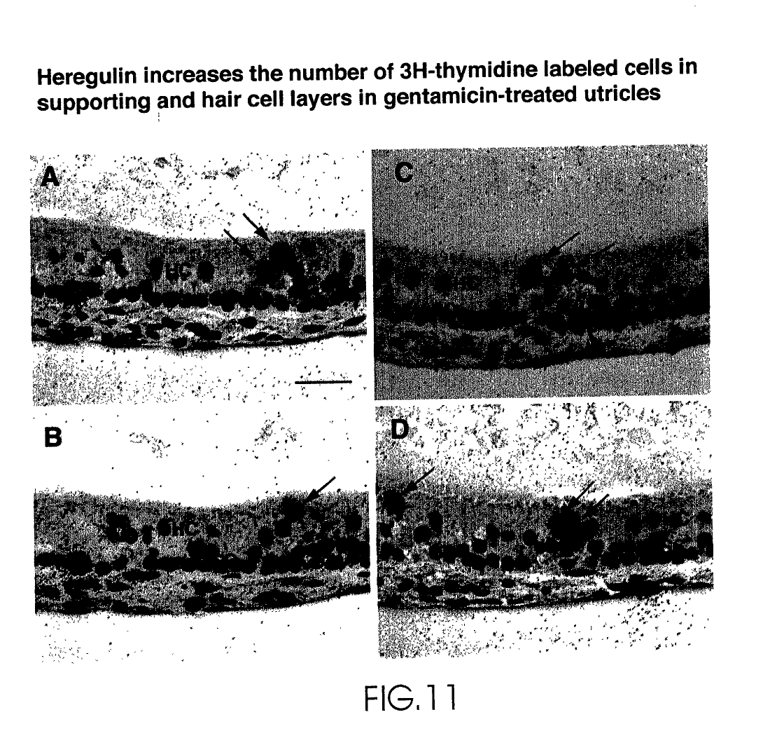

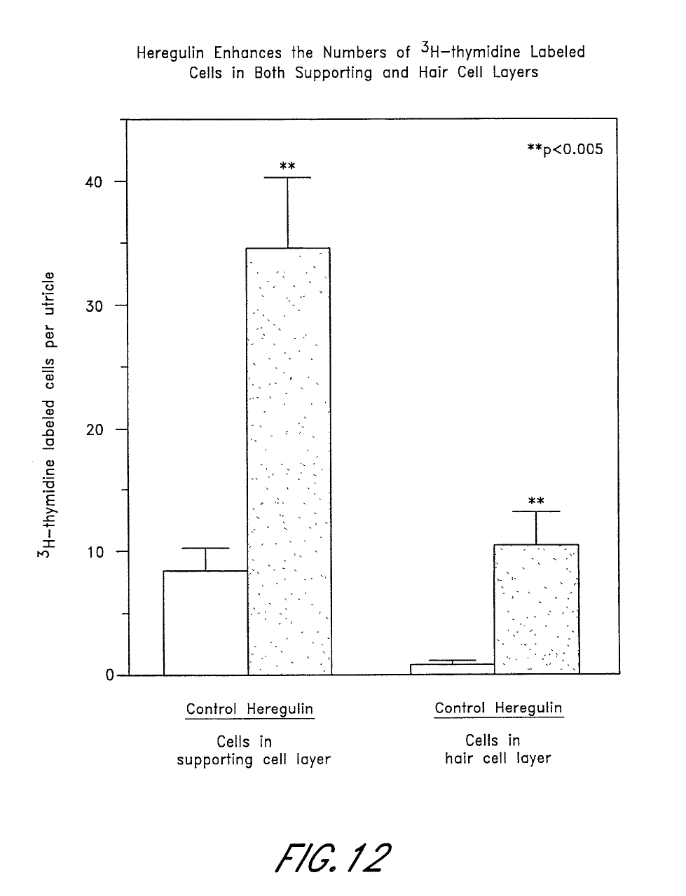

Organ cultures of utricular whole mounts were cultured 1-2 days after explant, then treated with gentamycin (1 mM) for two days, and then treated with heregulin (3 nM) for 11 days in the presence of tritiated thymidine. To determine the number of labeled cells, the tissue was fixed, sectioned and processed for autoradiography. In response to heregulin, compared to control cultures, an increase in the number of ³H-thymidine labeled cells in both the supporting cell layer (SC) and the hair cell layer (HC) was observed as shown in FIGS. 11A-D, which represent similarly treated samples. The cell count of ³H-thymidine labeled cells in both the supporting cell layer and in the hair cell layer increased significantly compared to control cultures lacking heregulin as shown in FIG. 12. The data is consistent with the data obtained in the utricular sheet cultures. And the data indicates that heregulin can act to increase inner-ear-supporting cell proliferation, which leads to hair cell generation, in instances following hair cell damage and injury.

### Example IV

Heregulin Acts through the Her2 Receptor

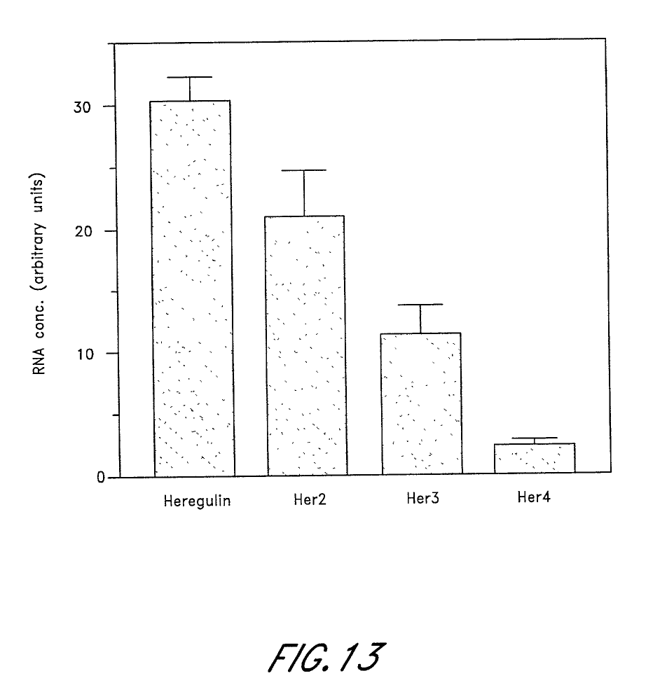

To provide further evidence that heregulin is a physiologically relevant factor and that it acts through a physiologically relevant receptor, the mRNA expression levels of heregulin and its receptors Her2, Her3 and Her4 in the hair cell and supporting cell layers of the rat utricular sensory epithelium were determined. RNA was extracted from the P3 utricle sheet cultures and also from UEC4 cells (a inner-ear-supporting cell line). Using TaqMan PCR analysis with appropriate gene-specific primers (Heid et al., Genome Research. 6(10):986-94 (1996)), it was observed that all four were expressed in the inner ear, however, heregulin and Her2 were expressed at a higher level than either Her3 or Her4 (see FIG. 13). Her4 was not expressed in the inner-ear-supporting cell line.

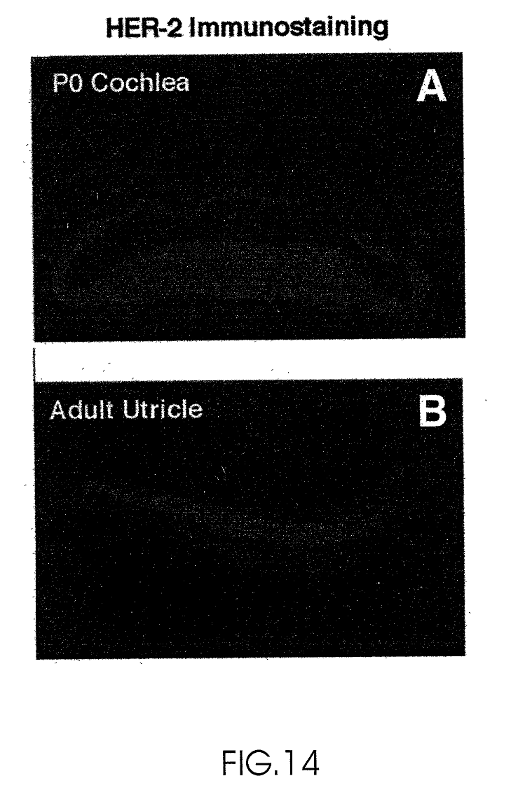

To determine that Her2 was indeed expressed at the protein level and to confirm its localization, fluorescently labeled anti-Her2 monoclonal antibody was used to immunostain rat P0 (day zero) cochlea and adult utricle. Her2 was localized to the hair cell and supporting cell sensory epithelium layers in the inner ear (see FIG. 14 A (cochlea) and FIG. 14B (utricle)). Anti-HER2 monoclonal antibodies 2C4 and 4D5 have been described elsewhere (Fendly et al. Cancer Research 50:1550-1558 (1990)). Consistent with this observation is that immunostaining with a heregulin antibody suggests that heregulin is expressed by hair cells of the inner ear.

Addition of neutralizing monoclonal antibodies against Her2, but not the addition of the immunoadhesin Her4-IgG, at saturating amounts to the utricular cultures, blocked the effects of heregulin. Thus, heregulin stimulates supporting cell proliferation and hence the generation of new hair cells by activating a Her2-mediated signaling pathway, but not a Her4-modiated pathway.

In addition, preliminary experiments with embryonic rat inner ear explant cultures show that heregulin affects hair cell differentiation by enhancing proliferation of hair cell progenitors. Rat E14 otocyst cultures treated with heregulin respond with an increase in the number of hair cell progenitor cells compared to untreated cultures. This is consistent with the adult tissue studies, indicating that heregulin stimulates the proliferation of cells that differentiate into hair cells.

### Example V

Heregulin Acts In Vivo to Enhance Inner Ear Supporting Cell Proliferation and Hair Cell Generation Following Ototoxic Injury and Acoustic Assault

Chinchillas are an accepted model to test the effects of factors and agents against or following hair cell damage or injury. Chinchillas can be treated with gentamicin, caboplatin or acoustic trauma. Preferably, at least five chinchillas are in each of the test and the control groups. The control group is treated with the ototoxic agent or acoustic assault and allowed to recover. Typically, four to six weeks is sufficient for recovery. The test group is treated with heregulin in addition to the injury. All animals will receive BrdU, preferably subcutaneous infusion, using minipumps, to label the dividing cells during the treatment period. Heregulin, or one of the heregulin factors as taught herein, will be administered to the inner ear. Minipumps can be used. The heregulin can be infused into the cochlea. After the treatment period, cochlea and utricular maculae are dissected out of the animals. The tissue is fixed and BrdU immunohistochemical labeling done. BrdU labeled cells in the inner ear sensory epithelium are counted. Cell counts from the two groups—are compared and analyzed statistically to determine the amount of enhancement of proliferation of supporting cells and new hair cell generation induced by the heregulin treatment.

Cited References

1. Acheson et al., Nature 374:450-453 (1995).

2. Adler et al., Assoc Res Otolaryngol Abstr, p4 (1996).

3. Anderson, Neuron 3, 1-12 (1989).

4. Balak et al., J Neurosci 10:2505-2512 (1990).

5. Beck et al., Neuron 14:717-730 (1995).

6. Bianchi et al., Assoc Res Otolaryngol Abstr, p108 (1995).

7. Cattaneo et al., Nature 347:762-765 (1990).

8. Corwin et al., Science 240:1772-1774 (1988).

9. Corwin et al., Assoc Res Otolaryngol Abstr, 18:87(1995).

10. Cotanche, Hear Res 30:181-196 (1987).

11. Cotanche et al., Curr Opinion Neurobiol 4:509-514 (1994).

12. Davis et al., Nature 372:263-266 (1994).

13. Ernfors et al., Nature 368:147-150 (1994).

14. Ernfors et al., Neuron 14:1153-1164 (1995).

15. Farinas et al., Nature 369:658-661 (1994).

16. Finley et al., Assoc Res Otolaryngol Abstr, p84 (1995).

17. Forge et al., Science 259:1616-1619 (1993).

18. Frenz et al, Development 120:415-424 (1994).

19. Fritzsch N et al., Auditory Neurosci 1:401-417 (1995).

20. Gao et al., Neuron 1: 269-277 (1988).

21. Gao et al., Neuron 6:705-715 (1991).

22. Gao et al., J Neurosci 15:2656-2667 (1995).

23. Ghosh et al., Neuron 15:89-103 (1995).

24. Gray et al., Assoc Res Otolaryngol Abstr, p198 (1996).

25. Holley et al (1995) J Neurocytol 24:853-864.

26. Johnson et al (1993) Adv Cancer Res 60:1-41.

27. Jones et al (1996) J Neurosci 16:649-662.

28. Kelley et al (1993) Development 119:1041-1053.

29. Kopke et al (1996) Assoc Res Otolaryngol Abstr, p198.

30. Lambert et al (1994) Laryngoscope 104:701-717.

31. Lee et al (1996) Hear Res 94:1-13.

32. Lefebvre et al (1994) Neuroreport 5:865-868.

33. Leon et al (1995) Endocrinology 136:3494-3503.

34. Li et al (1996) Assoc Res Otolatyngol Abstr, p7.

35. Li et al (1996) Endocrine, in press.

36. Low et al (1995) Assoc Res Otolaryngol Abstr, p200

37. Minichiello et al (1995) Development 121:4067-4075.

38. Miranda et al (1993) Proc Natl Acad Sci USA 90:6439-6443.

39. Neff et al (1993) J Neurobiol 24:1578-1588.

40. Oesterle et al (1996) Assoc Res Otolaryngol Abstr, p198.

41. Pirvola et al (1995) Proc Natl Acad Sci USA 92:9269-9273.

42. Rapheal et al (1992) J Neurocytol 21:663-671.

43. Reynold et al (1992) Science 255:1707-1710.

44. Ryals et al (1988) Science 240:1774-1776.

45. Sans et al (1982) J Comp Neurol 206:1-8.

46. Vicario-Abejon et al (1995) Neuron 15:105-114.

47. Warchol et al (1993) Hear Res 71:28-36.

48. Warchol et al (1993) Science 259:1619-1622.

49. Weisleder et al (1992) Exp Neurol 115:2-6.

50. Yamashita et al (1995) Proc Natl Acad Sci USA 92:3152-3155.

51. Zheng et al (1996) Eur J Neurosci, 8:1897-1905.

52. Zheng et al (1995a) J. Neurosci. 15:5079-5087.

53. Zheng et al (1995b) J. Neurobiol. 28:330-340.

54. Anniko et al (1986) Am J Otolaryngol 7:276-293.

55. Apfel et al (1991) Ann. Neurol. 29:87-89.

56. Ard et al. (1985). Neurosci. 16:151-170.

57. Au et al (1987) Biochem Biophys Acta 902:80-86.

58. Baired et al (1992) J. Neurosic. 12:619-634.

59. Barbacid et al. (1993). In Molecular Genetics of Nervous System Tumors. (New York: Wiley-Liss), pp123-136.

60. Barde et al (1982) EMBO J. 1:549-553.

61. Bareggi et al (1990). Pharmacol. Res. 22:635-644.

62. Barker et al (1994). Neuron 13:203-215.

63. Berggren et al., (1990) Acta Otolaryngol. (Stockh) 109:57-65.

64. Berkemeier et al., (1991) Neuron 7:857-866.

65. Boettcher et al., (1989) Hear Res 42: 129-142.

66. Boettcher et al., (1990) Arch Otolaryngol Head Neck Surg 116:681-684.

67. Boettcher et al, (1991) Am. J Otolaryngol 12: 33-47.

68. Carenza (1986). Gynecol. Oncol. 25:244-249.

69. Chao et al., (1986) Science 232:518-521.

70. Chao et al., (1992). Cell 68:995-997.

71. Clary et al., (1994). Proc. Natl. Acad. Sci. USA 91:11133-11137.

72. Clary et al (1994). Mol. Biol. Cell 5:549-563.

73. Cohen et al., (1994) J. Neurobiol. 25:953-959.

74. Cordon-Cardo et al (1991), Cell, 66, 173-183

75. Corwin et al (1991) Ann Rev Neurosci 14: 301-333.

76. Cotanche et al., (1994) Curr Opinion Neurobiol 4: 509-514.

77. Davies et al., (1993) Neuron 11:565-574.

78. Davies et al (1993b) J. Neurosci. 13:4961-4967.

79. Davies et al., (1986) Nature 319:497-502.

80. De Moura et al (1968) Arch Otolaryng 87:60-64.

81. Dublinet al., (1976): C. C. Thomas.

82. Duckert et al., J. Comp. Neurol. 331:75-96.

83. Ernfors et al (1990) Proc. Natl. Acad. Sci. USA 87:5454-5458.

84. Ernfors et al., (1994). Nature 368:147-150.

85. Ernfors et al., (1995) Assoc. Res Otolaryngol Abstr p190.

86. Escandon et al., (1994) J. Neurosci. 14:2954-2068.

87. Falbe-Hansen et al., (1941) Acta Otolaryng suppl 44:1-216.

88. Fariñas et al (1994). Nature 369:658-661.

89. Fischer et al (1994) Proc. Natl. Acad. Sci. USA 91:8607-8611.

90. Fleischman et al (1975) Pharmacol. 33:320-332.

91. Forge et al (1993) Science 259:1616-1619.

92. Fritzsch et al (1995). Assoc. Res. Otolaryngol Abstr. p190.

93. Furley et al (1990) Cell 61:157-170.

94. Gao et al (1991) Neuron 6:705-715.

95. Gao et al (1995b) Ann Neurol (1995) 38:30-37.

96. Gao et al (1995) J. Neurosci. 15:2656-2667.

97. Garner et al (1994) Neuron 13:457-472.

98. Gotz et al (1994) Nature 372:266-269.

99. Grotz et al., Eur. J. Biochem. 204:745-749 (1992)

100. Guild et al (1931). Acta Otolaryngol. (Stockh) 15:269-308.

101. Hefti et al (1986) J Neurosci 6:2155-2162.

102. Hinojosa et al (1987). J. Infect. Dis. 156: 449-455

103. Hohn et al (1990) Nature 344:339-341.

104. Hood et at., Contemporaty applications of neurobiology in human hearing assessment (Raven Press, New York, 1986).

105. Hulme, et al., E. C. and Birdsall, M. J. M., Strategy and Tactics in Receptor Binding Studies, p63-212 in Receptor-Ligand Interactions, Ed. E. C. Hulme

106. Hyman et al., (1991) Nature 350:230-233.

107. Hynes et al., (1994) J. Neurosci. Res. 37:144-154.

108. Ip et al., (1993) Neuron 110:137-149.

109. Ip et al (1992) Proc. Nat. Acad. Sci. USA 89:3060-3064.

110. Jarvis et al., (1966) J Laryngol 80: 318-320.

111. Jones et al., (1990) Proc. Natl. Acad. Sci. USA 87:8060-8064.

112. Kaplan et al., (1991) Science 252, 554-558

113. Kaplan et al., (1991) Nature 350:158-160.

114. Kelley et al., (1995) J Neurosci 15:3013-3026.

115. Kelley et al., (1993) Development 119:1041-1053.

116. Klein et al., (1991a) Cell 65:189-197.

117. Klein et al., (1991b) Cell 66:395-403.

118. Klein et al., (1990) Development 109:845-850.

119. Klein, et al., (1992) Neuron 8, 947-956

120. Klein, et al., (1989), EMBO J., 8, 3701-3709

121. Knusel et al., (1992) J Neurosci 12:4391-4402.

122. Koliatsos et al., (1993) Neuron 10:359-367.

123. Konings et al., (1994) Brain Res 640:195-204.

124. Kopf-Maier et al., (1992) Chem. Biol Interact 82:295-316.

125. Korsching, et al., (1993). J. Neurosi. 13:2739-2748.

126. Lamballe et al., (1991) Cell 66:967-979.

127. Lambert et al., (1994) Laryngoscope 104:701-718.

128. Lärkfors et al., (1993) Neurosci. 19:A278.14.

129. Leake et al., (1992 Hear Res 64:99-117.

130. Lefebvre et al., (1994) NeuroReport 5:865-868.

131. Lefebvre, et al., (1993). Science 260:692-695.

132. Lefebvre et al., (1991). Acta Otolaryngol (Stockh) 111:304-311.

133. Lefebvre et al., (1995) Science 267: 709-711.

134. Leibrock et al., (1989) Nature 341:149-152.

135. Levi-Montalcini et al., (1987) EMBO J. 6:1145-1154.

136. Lim et al., (1986) Am J Otolaryngol 7: 73-99.

137. Lippe et al., (1995) Assoc Res Otolaryngol Abstr. p84.

138. Maisonpierre et al., (1990) Science 247:1446-1451.

139. Martin-Zanca et al., (1989), Mol. Cell. Biol., 9, 24-33

140. McAlpine et al., (1990) Hear Res 47:191-204.

141. McCabe et al., (1965) Ann Otol 74: 312-324.

142. Mollman et al(1990) N. Engl. J. Med. 322:126-127.

143. Myers et al., (1965) Arch Otolaryngl Head Neck Surg 82: 483-493.

144. Nakai et al., (1982). Acta Otolaryngol 93:227-232.

145. Pirvola et al., (1992). Proc. Natl. Acad. Sci. USA 89:9915-9919.

146. Pryor et al., (1994) Chang LW, ed., Marcel Dekker, Inc. PP345-371.

147. Rastel et al., (1993) J Neurosci Methods 47:123-131.

148. Richardson et al., (1991) Hear Res 53:293-311.

149. Roelofs et al., (1984). Neurology 34:934-938.

150. Rosenthal et al., (1990) Neuron 4:767-773.

151. Rybak et al., (1986) In: Neurobiology of Hearing. Altschuler R A, Bobbin R P, Hoffman D W, Eds. Raven Press (New York) PP441-454.

152. Schacht et al., (1986) Hear Res 22: 297-304.

153. Schecterson et al.,(1994). Hear. Res. 73:92-100.

154. Scopes et al., R., Protein Purification, Springer-Verlag, NY (1982)

155. Sera et al., (1987). Scanning Microsc. 1:1191-1197.

156. Shelton et al., (1995). J. Neurosci. 15:477-491.

157. Siegal et al., (1990) Cancer 66:1117-1123.

158. Snider et al., (1994) Cell 77: 627-638.

159. Sobkowicz et al., (1975) J. neurocytol. 4:543-572.

160. Soppet et al., (1991) Cell 65:895-903.

161. Squinto et al., (1991) Cell 65:885-893.

162. Stadnicki et al., (1975). Cancer Chemother. Rep. 59:467-480.

163. Thompson et al., (1984). Cancer 54:1269-1275.

164. Tsoulfas et al.,(1993) Neuron 10:975-990.

165. Tsue et al., (1994a) Proc Natl Acad. Sci USA 91:1584-1588.

166. Tsue et al., (1994b) Head Neck Surg 111:281-301.

167. Valenzuela et al., (1993) Neuron 10: 963-974.

168. Vazquez et al., (1994) Anat. Embryol. 189:157-167.

169. Verdi, et al., (1994). Neuron 12:733-745.

170. VonBartheld et al., (1991). Development 113: 455-470.

171. Warchol et al., (1993). Science 259:1619-1622.

172. Weskamp et al., (1991). Neuron 6:649-663.

173. Wheeler et al., (1994). Hear. Res. 73:46-56.

174. Windebank et al., (1994) Neurology 44: 488-494.

175. Wittmaack et al., (1903). Pflueger Arch Ges Physiol 95:237.

176. Woodford et al., (1978) Ann Otol Rhinol Laryngol 87:117-127.

177. Yamashita et al., (1995) Proc Natl Acad Sci USA 92:3152-3155.

178. Yan et al., (1992) Nature 360:753-755.

179. Yan et al., (1994). Exper. Neurology 127:23-36.

180. Ylikoski et al., (1993). Hear. Res. 65:69-78.

Additonal Cited References

Anniko M, Sobin A (1986) Cisplatin: Evaluation of its ototoxic potential. Am J Otolaryngol 7:276-293.

Apfel S C, Lipton R B, Arezzo J C, Kessler J A (1991) Nerve growth factor prevents toxic neuropathy in mice. Ann. Neurol. 29:87-89.

Ard, M. D., Morest, D. K., and Hauger, S. H. (1985). Trophic interactions between the cochleovestibular ganglion of the chick embryo and its synaptic targets in culture. Neurosci. 16:151-170.

Au S, Weiner N D, Schacht J (1987) Aminoglycoside antibiotics preferentially increase permeability in phosphoinositide-containing membranes: a study with carboxyflurorescein in liposomes. Biochem Biophys Acta 902:80-86.

Baired D H, Hatten M E, Mason C A (1992) Cerebellar target neurons provide a stop signal for affrent neurite extension in vitro. J. Neurosic. 12:619-634.

Barbacid, M. (1993). The trk family of neurotrophin receptors: molecular characterization and oncogenic activation in hauman tumors. In Molecular Genetics of Nervous System Tumors. Levin, A. G. and Schmidek, H. H., eds. (New York: Wiley-Liss), pp123-136.

Barde Y A, Edgar D, Thoenen H (1982) Purification of a new neurotrophic factor from mammalian brain. EMBO J. 1:549-553.

Bareggi, R., Grill, V., Narducci, P., Zweyer, M., Tesei, L, and Russolo, M. (1990).Genetamicin ototoxicity: Histological and ulstructural alterations after transtympanic administration. Pharmacol. Res. 22:635-644.

Barker, P. A., and Shooter, E. M. (1994). Disruption of NGF binding to the low affinity neurotrophin receptor p75^{LNTR} reduces NGF binding to trkA on PC12 cells. Neuron 13:203-215.

Berggren, D, Anniko, M, Thornell, L.-E., Ramaekers, F. C. S., and Virtanem, I. (1990). Intermediate filament proteins in the embryonic inner ear of mice under normal conditions and after exposure to ototoxic drugs. Acta Otolaryngol. (Stockh) 109:57-65.

Berkemeier L R, Winslow J W, Kaplan D R, Nikolics K, Goeddel D V, Rosenthal A (1991) Neurotrophin-5: A novel neurotrophic factor that activates trk and trkB. Neuron 7:857-866.

Boettcher F A, Bancroft B R, Salvi R J, Henderson D (1989) Effects of sodium salicylate on evoked-response measures of hearing. Hear Res 42: 129-142.

Boettcher F A, Bancroft B R, Salvi R J, Henderson D (1990) Concentration of salicylate in serum and perilymph of the chinchilla. Arch Otolaryngol Head Neck Surg 116:681-684.

Boettcher F A, Salvi R J (1991) Salicylate ototoxicity: review and synthesis. Am. J Otolaryngol 12: 33-47.

Carenza, L., Villani, C., Framarino dei Malatesta, M. L., Prosperi Porta, R., Millefiorine, M., Antonini, G., Bolasco, P., Bandiera, G., and Marzetti, L. (1986). Peripheral neuropathy and ototoxicity of dichlorodiamineplatinum: instrumental evaluation. Gynecol. Oncol. 25:244-249.

Chao, M. V., Bothwell, M. A., Ross, A. H., Koprowski, H., Lanahan, A. A., Buck, C. R., and Sehgal, A. (1986). Gene transfer and molecular cloning of the human NGF receptor. Science 232:518-521.

Chao, M. V. (1992). Growth factor signalling: where is the specificity? Cell 68:995-997.

Clary, D. O., and Reichardt, L. F. (1994). An alternatively spliced form of the nerve growth factor receptor trkA confers an enhanced response to neurotrophin 3. Proc. Natl. Acad. Sci. USA 91:11133-11137.

Clary, D. O., Weskamp, G., Austin, L. R., and Reichardt, L. F. (1994). trkA cross-linking mimics neuronal responses to nerve growth factor. Mol. Biol. Cell 5:549-563.

Cohen A, Bray G M, Aguayo A J (1994) Neurotrophin-4/5 (NT-4/5) increases adult rat retinal ganglion cell survival and neurite outgrowth in vitro. J. Neurobiol. 25:953-959.

Cordon-Cardo, C., Tapley, P., Jing, S., Nanduri, V., O'Rourke, E., Lamballe, F., Kovary, K., Klein, R., Jones, K. R., Reichhardt, L. F. and Barbacid, M. (1991), Cell, 66,173-183

Corwin J T, Warchol M E (1991) Auditory hair cells: structure, function, development, and degeneration. Ann Rev Neurosci 14: 301-333.

Cotanche D A, Lee K H (1994) Regeneration of hair cells in the vestibulocochlear system of birds and mammals. Curr Opinion Neurobiol 4: 509-514.

Davies, A. M., Lee, K. F., and Jaenisch, R. (1993). p75-deficient trigeminal sensory neurons have an altered response to NGF but not to other neurotrophins. Neuron 11:565-574.

Davies A M, Horton A, Burton L E, Schmeizer C, Vandlen R, Rosenthal A (1993b) Neurotrophin-4/5 is a mammalian-specific survival factor for distinct populations of sensory neurons. J. Neurosci. 13:4961-4967.

Davies, A. M., Thoenen, H., and Barde, Y.-A. (1986). Different factors from the central nervous system and peripheral regulate the survival of sensory neurons. Nature 319:497-502.

De Moura L F P, Hayden R C (1968) Salicylate ototoxicity. Arch Otolaryng 87:60-64.

Dublin W B (1976) Fundamentals of sensorineural auditory pathology. Springfield, Ill.: C. C. Thomas.

Duckert, L. G., and Rubel, E. W. (1994). Morphological correlates of the functional recovery in the chicken inner ear after gentamicin treatment. J. Comp. Neurol. 331:75-96.

Ernfors P, Ibanez C F, Ebendal T, Olson L, Persson H (1990) Molecular cloning and neurotrophic activities of a protein with structural similarities to nerve growth factor: developmental and topographic expression in the brain. Proc. Natl. Acad. Sci. USA 87:5454-5458.

Ernfors, P., Lee, K.-F., and Jaenisch, R. (1994). Mice lacking brain-derived neurotrophic factor develop with sensory deficits. Nature 368:147-150.

Ernfors P, Loring J, Jaenisch R, Van De Water T R (1995) Function of neurotrophins in the auditory and vestibular systems: Analysis of BDNF and NT-3 gene knockout mice. Assoc. Res Otolaryngol Abstr p190.

Escandon E, Soppet D, Rosenthal A, Mendoza-Ramierz J-L, Szonyi E, Burton L E, Henderson C E, Parada L F, Nikolics K (1994) Regulation of neurotrophin receptor expression during embryonic and postnatal development. J. Neurosci. 14:2954-2068.

Falbe-Hansen J (1941) Clinical and experimental histological studies on effects of salicylate and quinine on the ear. Acta Otolaryng suppl 44: 1-216.

Fariñas, I., Jones, K. R., Backus, C., Wang, X.-Y., and Reichardt, L. F. (1994). Severe sensory and sympathetic deficits in mice lacking neurotrophins-3. Nature 369:658-661.

Fischer W, Sirevaag A, Wiegand S J, Lindsay R M, Björklund A (1994) Reversal of spatial memory impairments in aged rats by nerve growth factor and neurotrophins 3 and 4/5 but not by brain-derived neurotrophic factor. Proc. Natl. Acad. Sci. USA 91:8607-8611.

Fleischman, R. W., Stadnicki, S. W., Ethier, M. F., and Schaeppi, U. (1975). Ototoxicity of cis-dichlorodiammine platinum (II) in the guinea pig. Toxicol Appl. Pharmacol. 33:320-332.

Forge A, Li L, Corwin J T, Nevill G (1993) Ultrastructural evidence for hair cell regeneration in the mammalian inner ear. Science 259:1616-1619.

Fritzsch, B., Smyene, R., Fagan, A., Selos-Santiago, I. (1995). Mice homologous for a non-functional trk-B receptor lack selectively in the innervation of semicircular canals. Assoc. Res. Otolaryngol. Abstr. p190.

Furley A, Morton S B, Malano D, Karagogeos, Dodd J, Jessell T M (1990) The axonal glycoprotein TAG-1 is an immunoglobin superfamily member with neurite outgrowth-promoting activity. Cell 61:157-170.

Gao W-Q, Heitz N, Hatten M E (1991) Cerebellar granule cell neurogenesis is regulated by cell-cell interactions in vitro. Neuron 6:705-715.

Gao W-Q, Dybdal N, Shinsky N, Murnane A, Schmelzer C, Siegel M, Keller G, Hefti F, Phillips H S, Winslow J W (1995b) Neurotrophin-3 reverses experimetal cisplatin-induced peripheral sensory neuropathy. Ann Neurol (1995) 38:30-37.

Gao, W.-Q., Zheng, J. L., and Karihaloo, M. (1995). Neurotrophin-4/5 (NT-4/5) and brain-derived neurotrophic factor (BDNF) act at later stages of cerebellar granule cell differentiation. J. Neurosci. 15:2656-2667.

Garner A S, Large T H (1994) Isoforms of the avian trkC receptor: A novel kinase insertion dissociates transformation and process outgrowth from survival. Neuron 13:457-472.

Gotz, R., Koster, R., Winkler, C., Raulf, F., Lottspelch, F., Scharti, M., and Thoenen, H. (1994). Neurotrophin-6 is a new member of the nerve growth factor family. Nature 372:266-269.

Grotz et al., Eur. J. Biochem. 204:745-749 (1992)

Guild S, Cowe S, Bunch C, Polvogt (1931) Correlations of differences in sensory of innervation of the organ of Corti with differences in the acuity of hearing, including evidences as to the location in the human cochlea of receptors for certain tones. Acta Otolaryngol. (Stockh) 15:269-308.

Hefti F (1986) Nerve growth factor (NGF) promotes survival of septal cholinergic neurons after fimbrial transections. J Neurosci 6:2155-2162.

Hinojosa, R., and Lerner, S. A. (1987). Cochlear neural degeration without hair cell loss in two patients with aminoglycoside ototoxicity. J. Infect Dis. 156: 449-455

Hohn A, Leibrock J, Bailey K, Barde Y A (1990) Identification and characterization of a novel member of the nerve growth factor/brain-derived neurotrophic factor family. Nature 344:339-341.

Hood J L, Berlin C I, Ed., Contemporary applications of neurobiology in human hearing assessment (Raven Press, New York, 1986).

Hulme, E. C. and Birdsall, M. J. M., Strategy and Tactics in Receptor Binding Studies, p63-212 in Receptor-Ligand Interactions, Ed. E. C. Hulme

Hyman C, Hofer M, Barde Y A, Juhasz M, Yancopoulos G D, Squinto S P, Lindsay R M (1991) BDNF is a neurotrophic factor for dopaminergic neurons of the substantia nigra. Nature 350:230-233.

Hynes M A, Poulsen K, Armanini M, Berkemeier L, Phillips H, Rosenthal A (1994) Neurotrophin-4/5 is a survival factor for embryonic midbrain dopaminergic neurons in enriched cultures. J. Neurosci. Res. 37:144-154.

Ip N Y, Stitt T N, Tapley P, Klein R, Glass D J, Fandl J, Greene L A, Barbacid M, Yancopoulos G D (1993) Similarities and differences in the way neurotrophins interact with the trk receptors in neuronal and nonneuronal cells. Neuron 110:137-149.

Ip N Y, Ibáñez C F, Nye S H, McClain J, Jones P F, Gies D R, Belluscio L, LeBeau M M, Espinso III R Squinto S P, Persson H, Yancopoulos G D (1992) Mammalian neurotrophin-4: Structure, chromosomal localization, tissue distribution, and receptor specificity. Proc. Nat. Acad. Sci. USA 89:3060-3064.

Jarvis J F (1966) A case of unilateral permanent deafness following acetyl salicylic acid. J Laryngol 80: 318-320.

Jones K R, Reichardt L F (1990) Molecular cloning of a human gene that is a member of the nerve growth factor family. Proc. Natl. Acad. Sci. USA 87:8060-8064.

Kaplan, D. R., Hempstead, B., Martin-Zanca, D., Chao, M., and Parada, L. F. (1991) Science 252, 554-558

Kaplan D R, Martin Z D, Parada L F (1991) Tyrosine phosphorylation and tyrosine kinase activity of the trk proto-oncogene product induced by NGF. Nature 350:158-160.

Kelley M W, Telreja D R, Corwin J T (1995) Replacement of hair cells after microbeam irradiation in cultured organs of Corti from embryonic and neonatal mice. J Neurosci 15:3013-3026.

Kelley M W, Xu X-M, Wagner M A, Warchol M E, Corwin J T (1993) The developing organ of Corti contains retinoic acid and forms supernumerary hair cells in response to exogenous retinoic acid in culture. Development 119:1041-1053.

Klein R, Jing S Q, Nanduri V, O'Rourke E, Barbacid M (1991a) The trk proto-oncogene encodes a receptor for nerve growth factor. Cell 65:189-197.

Klein R, Nanduri V, Jing S A, Lamballe F, Tapley P, Bryant S, Cordon-Cardo C, Jones K R, Reichardt L F, Barbacid M (1991b) The trkB tyrosine protein kinase is a receptor for brain-derived neurotrophic factor and neurotrophin-3. Cell 66:395-403.

Klein R, Martin-Zanca D, Barbacid M, Parada L F (1990) Expression of the tyrosine kinase receptor gene trkB is confined to the murine embryonic and adult nervous system. Development 109:845-850.

Klein, R., Lamballe, F., Bryant, S., and Barbacid, M. (1992) Neuron 8, 947-956

Klein, R., Parada, L. F., Coulier, F. and Barbacid, M. (1989), EMBO J., 8, 3701-3709

Knusel B, Beck K D, Winslow J W, Rosenthal A, Burton L E, Widmer H R, Nikolics K, Hefti F (1992) Brain-derived neurotrophic factor administration protects basal forebrain cholinergic but not nigral dopaminergic neurons from degenerative changes after axotomy in the adult rat brain. J Neurosci 12:4391-4402.

Koliatsos V E, Clatterbuck R E, Winslow J W, Cayouette M H, Price D L (1993) Evidence that brain-derived neurotrophic factor is a trophic factor for motor neurons in vivo. Neuron 10:359-367.

Konings P N M, Makkink W K, van Delft A M L, Ruigt G S F (1994) Reversal by NGF of cytostatic drug-induced reduction of neurite outgrowth in rat dorsal root ganglia in vitro. Brain Res 640:195-204.

Kopf-Maier P, Muhlhausen S K (1992) Changes in the cytoskeleton pattern of tumor cells by cisplatin in vitro. Chem. Biol Interact 82:295-316.

Korsching, S. (1993). The neurotrophic factor concept: A reexamination. J. Neurosci. 13:2739-2748.

Lamballe F, Klein R, Barbacid M (1991) trkC, a new member of the trk family of tyrosine protein kinase, is a receptor for neurotrophin-3. Cell 66:967-979.

Lambert P R (1994) Inner ear hair cell regeneration in a mammal: identification of a triggering factor. Laryngoscope 104:701-718.

Lärkfors L, Ebendal T, Lindsay R M, Alderson R F (1993) Effects of neurotrophins on rat embryonic cerebellar purkinje cells in vitro. Abstr. Soc. Neurosci. 19:A278.14.

Leake P A, Snyder R L, Hradek G T, Rebscher S J (1992) Chronic intracochlear electrical stimulation in neonatally deafened cats: effects of intensity and stimulating electrod location. Hear Res 64:99-117.

Lefebvre P P, Malgrange B, Staecher H, Moghadass M, Van De Water T R, Moonen G (1994) Neurotrophins affect survival and neuritogenesis by adult injured auditory neurons in vitro. NeuroReport 5:865-868.

Lefebvre, P. P., Malgrange, B., Staecker, H., Moonen, G. and Van De Water, T. R. (1993). Retinoic acid stimulates regeneration of mammalian auditory hair cells. Science 260:692-695.

Lefebvre, P. P., Van De Water, T. R., Represa, J., Liu, W., Bernd, P., Modlin, S., Moonen, G., and Mayer, M. B. (1991). Temporal pattern of nerve growth factor (NGF) binding in vivo and the in vitro effects of NGF on cultures of developing auditory and vestibular neurons. Acta Otolaryngol (Stockh) 111:304-311.

Lefebvre P P, Malgrange B, Moonen G, Van De Water T R (1995) Response to: Regeneration and mammalian auditory hair cells. Science 267: 709-711.

Leibrock J, Lottspeich F, Hohn A, Hofer M, Hengerer B, Masiakowski P, Thoenen H, Barde Y A (1989) Molecular cloning and expression of brain-derived neurotrophic factor. Nature 341:149-152.

Levi-Montalcini R (1987) The nerve growth factor: thirty-five years later. EMBO J. 6:1145-1154.

Lim D J (1986) Effects of noise and ototoxic drugs at the cellular level in the cochlea: A review. Am J Otolaryngol 7: 73-99.

Lippe W R, Hathaway O, Parlotz D (1995) Loss of avian spiral ganglion neurons following aminoglycosie-induced hair cell loss and regeneration. Assoc Res Otolaryngol Abstr. p84.

Maisonpierre P C, Belluscio L, Squinto S, Ip N Y, Furth M E, Lindsay R M, Yancopoulos G D (1990) Neurotrophin-3: a neurotrophic factor related to NGF and BDNF. Science 247:1446-1451.

Martin-Zanca, D., Oskam, R., Mitra, G., Copeland, T. and Barbacid, M. (1989), Mol.Cell. Biol., 9, 24-33

McAlpine D, Johnstone B M (1990) The ototoxic mechanism of cisplatin. Hear Res 47:191-204.

McCabe P, Dey F (1965) The effects of aspirin upon auditory sensitivity. Ann Otol 74: 312-324.

Mollman J E (1990) Cisplatin neurotoxicity. N. EngI. J. Med. 322:126-127.

Myers E N, Berstaein J M (1965) Salicylate ototoxicity: A clinical and experimental study. Arch Otolaryngl Head Neck Surg 82: 483-493.

Nakai, Y., Konishi, K., Chang, K. C., Ohashi, K., Morisaki, N., Minowa, Y., and Morimoto, A. (1982). Ototoxicity of the anticancer drug cisplatin. Acta Otolaryngol 93:227-232.

Pirvola, U., Ylikoski, J., Palgi, J., Lehtonen, E., Arumäe, U., and Saarma, M. (1992). Brain-derived neurotrophic factor and neurotrophin 3 mRNAs in the peripheral target fields of developing inner ear ganglia. Proc. Natl. Acad. Sci. USA 89:9915-9919.

Pryor G (1994) Assessment of auditory dysfunction. In Principle of Neurotoxicology. Chang L W, ed., Marcel Dekker, Inc. PP345-371.

Rastel D, Abdouh A, Dahl D, Roman R (1993) An original organotypic culture method to study the organ of Corti of the newborn rat in vitro. J Neurosci Methods 47:123-131.

Richardson G P, Russell I J (1991) Cochlear cultures as a model system for studying aminoglycoside ototoxicity. Hear Res 53:293-311.

Roelofs, R. I., Hrushesky, W., Rogin, J., and Rosenberg, L. (1984). Peripheral sensory neuropathy and cisplatin chemotherapy. Neurology 34:934-938.

Rosenthal A, Goeddel D, Nguyen T, Lewis M, Shih A, Laramee G R, Nikolics K, Winslow J W (1990) Primary structure and biological activity of a novel human neurotrophic factor. Neuron 4:767-773.

Rybak L P (1986) Ototoxic mechanisms. In: Neurobiology of Hearing. Altschuler R A, Bobbin R P, Hoffman D W, Eds. Raven Press (New York) PP441-454.

Schacht J (1986) Molecular mechanisms of drug-induced hearing loss. Hear Res 22: 297-304.

Schecterson, L. C., and Bothwell, M. (1994). Neurotrophin and neurotrophin receptor mRNA expression in developing inner ear. Hear. Res. 73:92-100.

Scopes, R., Protein Purification, Springer-Verlag, NY (1982)

Sera, K., Harada, Y., Tagashira, N., Suzuki, M., Hirakawa, K., and Ohya, T. (1987). Morphological changes in the vestibular epithelia and ganglion induced by ototoxic drug. Scanning Microsc. 1:1191-1197.

Shelton, D. L., Sutherland, J., Gripp, J., Camertato, T., Armanini, M. P., Phillips, H. S., Carroll, K., Spencer, S. D., and Levinson, A. D. (1995). Human trks: Molecular cloning, tissue distribution, and expression of extracellular domain immunoadhesins. J. Neurosci. 15:477-491.

Siegal T, Haim N (1990) Cisplatin-induced peripheral neuropathy. Cancer 66:1117-1123.

Snider, W. D. (1994), Functions of the neurotrophins during nervous system development: What the knockouts are teaching us. Cell 77: 627-638.

Sobkowicz H M, Bereman B, Rose J E (1975) Organotypic develoment of the organ of Corti in culture. J. neurocytol. 4:543-572.

Soppet D, Escandon E, Maragos J, Middlemas D S, Reid S W, Blair J, Burton L E, Stanton B R, Kaplan D R, Hunter T, Nikolics K, Parada L F (1991) The neurotrophic factors brain-derived neurotrophic factor and neurotrophin-3 are ligands for the trkB tyrosine kinase receptor. Cell 65:895-903.

Squinto S P, Stitt T N, Aldrich T H, Davis S, Bianco S M, Radziejewski C, Glass D J, Masiakowski P, Furth M E, Valenzuela D M, DiStefano P S, Yancopoulos G D (1991) trkB encodes a functional receptor for brain-derived neurotrophic factor and neurotrophin-3 but not nerve growth factor. Cell 65:885-893.

Stadnicki, S. W., Fleischman, R. W., Schaeppi, U., and Merriam, P. (1975). Cis-dichlorodiammineplatinum (II) (NSC-119875): Hearing loss and other toxic effects in rhesus monkeys. Cancer Chemother. Rep. 59:467-480.

Thompson, S. W., Davis, L. E., Kornfeld, M., Hilgers, R. D., and Standefer, J. C. (1984). Cisplatin neuropathy. Cancer 54:1269-1275.

Tsoulfas P, Soppet D, Escandon E, Tessarollo L, Mendoza-Ramirez J-L, Rosenthal A, Nikolics K, Parada L F (1993) The rat trkC locus encodes multiple neurogenic receptors that exhibit differential response to neurotrophin-3 in PC12 cells. Neuron 10:975-990.

Tsue T T, Oesterle E C, Rubel E W (1994a) Diffusible factors regulate hair cell regeneration in the avian inner ear. Proc Natl Acad. Sci USA 91:1584-1588.

Tsue T T, Oesterle E C, Rubel E W (1994b) Hair cell regeneration in the inner ear. Otolaryngol. Head Neck Surg 111:281-301.

Valenzuela D M, Maisonpierre P C, Glass D J, Rojas E, Nunez L, Kong Y, Gies D R, Stitt T N, Ip N Y, Yancopoulos G D (1993) Alternative forms of rat trkC with different functional capabilities. Neuron 10: 963-974.

Vazquez E, Van De Water T R, Del Valle M, Veta J A, Staecker H, Giráldez F, Represa J (1994) Pattern of trkB protein-like immunoreactivity in vivo and the in vitro effects of brain-derived neurotrophic factor (BDNF) on developing cochlear and vestibular neurons. Anat. Embryol. 189:157-167.

Verdi, J. M., Birren, S. J., Ibanez, C. F., Persson, H., Kaplan, D. R., Benedett, M., Chao, M. V., and Anderson, D. J. (1994). p75^{LNGFR} regulates trk signal transduction and NGF-induced neuronal differentiation in MAII cells. Neuron 12:733-745.

Von Bartheld, C. S., Patterson, S. L., Heuer, J. G., Wheeler, E. F., Bothwell, M., and Rubel, E. W. (1991). Expression of nerve growth factor (NGF) receptors in the developing inner ear of chick and rat. Development 113: 455-470.

Warchol, M. E., Lambert, P. R., Goldstein, B. J., Forge, A., and Corwin, J. T. (1993). Regenerative proliferation in inner ear sensory epithelia from adult Guinea pigs and humans. Science 259:1619-1622.

Weskamp, G., and Reichardt, L. F. (1991). Evidence that biological activity of NGF is mediated through a novel subclass of high affinity receptors. Neuron 6:649-663.

Wheeler, E. F., Bothwell, M., Schecterson, L. C., and Von Bartheld, C. S. (1994). Expression of BDNF and NT-3 mRNA in hair cells of the organ of corti: Quantitative analysis in developing rats. Hear. Res. 73:46-56.

Windebank A J, Smith A G, Russell J W (1994) The effect of nerve growth factor, ciliary neurotrophic factor, and ACTH analogs on cisplatin neurotoxicity in vitro. Neurology 44: 488-494.

Wittmaack K (1903) Beitrage zur Kenntnis der Wirkung des Chinins auf das Gehoerorgan. Pflueger Arch Ges Physiol 95:237.

Woodford C M, Henderson D, Hamernik R P (1978) Effects of combinations of sodium salicylate and noise on the auditory threshold. Ann Otol Rhinol Laryngol 87:117-127.

Yamashita H, Oesterle E C (1995) Induction of cell proliferation in mammalian inner-ear sensory epithelia by transforing growth factor a and epidermal growth factor. Proc Natl Acad Sci USA 92:3152-3155.

Yan Q, Elliott J L, Snider W D (1992) Brain-derived neurotrophic factor rescues spinal motoneurons from axotomy induced cell death. Nature 360:753-755.

Yan, Q., Matheson, C., Sun, J., Radeke, M. J., Feinstein, S. C., and Miller, J. A. (1994). Distribution of intracerebral ventricularly administered neurotrophins in rat brain and its correlation with trk receptor expression. Exper. Neurology 127:23-36.

Ylikoski, J., Pirvola, U., Moshnyakov, M., Palgi, J., Arumae, U., and Saarma, M. (1993). Expression patterns of neurotrophin and their receptor mRNAs in the rat inner ear. Hear. Res. 65:69-78.

We claim:

1. A method of inducing hair cell generation or inner-ear-supporting cell growth, regeneration, and/or proliferation, comprising contacting an inner-ear-supporting cell which expresses HER2 and/or HER3 receptors with an effective amount of an isolated ligand which activates HER2 and/or HER3 receptors or a combination thereof.

2. The method of claim 1, wherein the activating ligand is a heregulin polypeptide, heregulin variant, heregulin agonist antibody or fragment thereof capable of binding to the HER2 or HER3 receptor.

3. The method of claim 2, wherein the activating ligand is human heregulin or a fragment thereof.

4. The method of claim 2, wherein the activating ligand is selected from the group consisting of HRG-α, -β1, -β2, -β2-like, and -β3 and fragments thereof.

5. The method of claim 2, wherein the activating ligand is γ-HRG or a fragment thereof.

6. The method of claim 2, wherein the activating ligand is recombinant human heregulin or a fragment thereof.

7. The method of claim 2, wherein the supporting cell is in cochlear implant.

8. The method of claim 1, wherein the activating ligand is administered at a daily dose of about 1 μg/kg to 100 mg/kg.

9. The method of claim 2, wherein the activating ligand is an agonist antibody.

10. The method of claim 1, wherein the contacting is by administration to a patient in need thereof.

11. The method of claim 6, wherein the heregulin is rHRG-β1-177-244.

12. The method of claim 1, wherein the inner-ear-supporting cell is in the utricle or cochlea.

13. The method of claim 1 wherein the inner-ear-supporting cell expresses HER2, HER3, or both.

14. A method of increasing the number of inner ear supporting cells, comprising administering to a patient in need thereof an effective amount of an isolated HER2 and/or HER3 activating ligand.

15. The method of claim 14, wherein the activating ligand is a heregulin polypeptide, heregulin variant, heregulin agonist antibody or fragment thereof capable of binding to the HER2 and/or HER3 receptor.

16. A method of treating a hair cell related hearing disorder, comprising administering to a patient in need thereof an effective amount of an isolated HER2 and/or HER3 activating ligand.

17. The method of claim 16, wherein the activating ligand is a heregulin polypeptide, heregulin variant, heregulin agonist antibody or fragment thereof capable of binding to the HER2 and/or HER3 receptor.

18. A method, comprising the steps of:
(a) obtaining an inner-ear-supporting cell sample from a mammal;
(b) contacting the sample with a ligand which activates HER2 or HER3 or a combination thereof to induce growth and/or proliferation of inner-ear-supporting cells in the sample and to obtain an expanded sample; and
(c) re-introducing the expanded sample into the mammal.


SEQUENCE LISTING (14 sequences)

SEQ ID NO: 1 - protein, 669 aa, Homo sapiens
ARAPQRGRSLSPSRDKLFPNPIRALGPNSPAPRAVRVERSVSGEMSERKEGRGKGKGKKKERGSGKKPESAAGSQSPALPPRLKEMKSQESAAGSKLVLRCETSSEYSSLRFKWFKNGNELNRKNKPQNIKIQKKPGKSELRINKASLADSGEYMCKVISKLGNDSASANITIVESNEIITGMPASTEGAYVSSESPIRISVSTEGANTSSSTSTSTTGTSHLVKCAEKEKTFCVNGGECFMVKDLSNPSRYLCKCQPGFTGARCTENVPMKVQNQEKAEELYQKRVLTITGICIALLVVGIMCVVAYCKTKKQRKKLHDRLRQSLRSERNNMMNIANGPHHPNPPPENVQLVNQYVSKNVISSEHIVEREAETSFSTSHYTSTAHHSTTVTQTPSHSWSNGHTESILSESHSVIVMSSVENSRHSSPTGGPRGRLNGTGGPRECNSFLRHARETPDSYRDSPHSERYVSAMTTPARMSPVDFHTPSSPKSPPSEMSPPVSSMTVSMPSMAVSPFMEEERPLLLVTPPRLREKKFDHHPQQFSSFHHNPAHDSNSLPASPLRIVEDEEYETTQEYEPAQEPVKKLANSRRAKRTKPNGHIANRLEVDSNTSSQSSNSESETEDERVGEDTPFLGIQNPLAASLEATPAFRLADSRTNPAGRFSTQEEIQ

SEQ ID NO: 2 - DNA, 2226 bp, Homo sapiens
  CDS (2)...(2008)
gggcgcgagcgcctcagcgcggccgctcgctctccccctcgagggacaaacttttcccaaacccgatccgagcccttggaccaaactcgcctgcgccgagagccgtccgcgtagagcgctccgtctccggcgagatgtccgagcgcaaagaaggcagaggcaaagggaagggcaagaagaaggagcgaggctccggcaagaagccggagtccgcggcgggcagccagagcccagccttgcctccccgattgaaagagatgaaaagccaggaatcggctgcaggttccaaactagtccttcggtgtgaaaccagttctgaatactcctctctcagattcaagtggttcaagaatgggaatgaattgaatcgaaaaaacaaaccacaaaatatcaagatacaaaaaaagccagggaagtcagaacttcgcattaacaaagcatcactggctgattctggagagtatatgtgcaaagtgatcagcaaattaggaaatgacagtgcctctgccaatatcaccatcgtggaatcaaacgagatcatcactggtatgccagcctcaactgaaggagcatatgtgtcttcagagtctcccattagaatatcagtatccacagaaggagcaaatacttcttcatctacatctacatccaccactgggacaagccatcttgtaaaatgtgcggagaaggagaaaactttctgtgtgaatggaggggagtgcttcatggtgaaagacctttcaaacccctcgagatacttgtgcaagtgccaacctggattcactggagcaagatgtactgagaatgtgcccatgaaagtccaaaaccaagaaaaggcggaggagctgtaccagaagagagtgctgaccataaccggcatctgcatcgccctccttgtggtcggcatcatgtgtgtggtggcctactgcaaaaccaagaaacagcggaaaaagctgcatgaccgtcttcggcagagccttcggtctgaacgaaacaatatgatgaacattgccaatgggcctcaccatcctaacccaccccccgagaatgtccagctggtgaatcaatacgtatctaaaaacgtcatctccagtgagcatattgttgagagagaagcagagacatccttttccaccagtcactatacttccacagcccatcactccactactgtcacccagactcctagccacagctggagcaacggacacactgaaagcatcctttccgaaagccactctgtaatcgtgatgtcatccgtagaaaacagtaggcacagcagcccaactgggggcccaagaggacgtcttaatggcacaggaggccctcgtgaatgtaacagcttcctcaggcatgccagagaaacccctgattcctaccgagactctcctcatagtgaaaggtatgtgtcagccatgaccaccccggctcgtatgtcacctgtagatttccacacgccaagctcccccaaatcgcccccttcggaaatgtctccacccgtgtccagcatgacggtgtccatgccttccatggcggtcagccccttcatggaagaagagagacctctacttctcgtgacaccaccaaggctgcgggagaagaagtttgaccatcaccctcagcagttcagctccttccaccacaaccccgcgcatgacagtaacagcctccctgctagccccttgaggatagtggaggatgaggagtatgaaacgacccaagagtacgagccagcccaagagcctgttaagaaactcgccaatagccggcgggccaaaagaaccaagcccaatggccacattgctaacagattggaagtggacagcaacacaagctcccagagcagtaactcagagagtgaaacagaagatgaaagagtaggtgaagatacgcctttcctgggcatacagaaccccctggcagccagtcttgaggcaacacctgccttccgcctggctgacagcaggactaacccagcaggccgcttctcgacacaggaagaaatccaggccaggctgtctagtgtaattgctaaccaagaccctattgctgtataaaacctaaataaacacatagattcacctgtaaaactttattttatataataaagtattccaccttaaattaaacaatttattttattttagcagttctgcaaatagaaaacaggaaaaaaacttttataaattaaatatatgtatgtaaaaatgaaaaaaaaaaaaaaaa

SEQ ID NO: 3 - protein, 675 aa, Homo sapiens
DKLFPNPIRALGPNSPAPRAVRVERSVSGEMSERKEGRGKGKGKKKERGSGKKPESAAGSQSPALPPQLKEMKSQESAAGSKLVLRCETSSEYSSLRFKWFKNGNELNRKNKPQNIKIQKKPGKSELRINKASLADSGEYMCKVISKLGNDSASANITIVESNEIITGMPASTEGAYVSSESPIRISVSTEGANTSSSTSTSTTGTSHLVKCAEKEKTFCVNGGECFMVKDLSNPSRYLCKCPNEFTGDRCQNYVMASFYKHLGIEFMEAEELYQKRVLTITGICIALLVVGIMCVVAYCKTKKQRKKLHDRLRQSLRSERNNMMNIANGPHHPNPPPENVQLVNQYVSKNVISSEHIVEREAETSFSTSHYTSTAHHSTTVTQTPSHSWSNGHTESILSESHSVIVMSSVENSRHSSPTGGPRGRLNGTGGPRECNSFLRHARETPDSYRDSPHSERYVSAMTTPARMSPVDFHTPSSPKSPPSEMSPPVSSMTVSMPSMAVSPFMEEERPLLLVTPPRLREKKFDHHPQQFSSFHHNPAHDSNSLPASPLRIVEDEEYETTQEYEPAQEPVKKLANSRRAKRTKPNGHIANRLEVDSNTSSQSSNSESETEDERVGEDTPFLGIQNPLAASLEATPAFRLADSRTNPAGRFSTQEEIQARLSSVIANQDPIAV

SEQ ID NO: 4 - DNA, 2199 bp, Homo sapiens
  CDS (3)...(2027)
gggacaaacttttcccaaacccgatccgagcccttggaccaaactcgcctgcgccgagagccgtccgcgtagagcgctccgtctccggcgagatgtccgagcgcaaagaaggcagaggcaaagggaagggcaagaagaaggagcgaggctccggcaagaagccggagtccgcggcgggcagccagagcccagccttgcctccccaattgaaagagatgaaaagccaggaatcggctgcaggttccaaactagtccttcggtgtgaaaccagttctgaatactcctctctcagattcaagtggttcaagaatgggaatgaattgaatcgaaaaaacaaaccacaaaatatcaagatacaaaaaaagccagggaagtcagaacttcgcattaacaaagcatcactggctgattctggagagtatatgtgcaaagtgatcagcaaattaggaaatgacagtgcctctgccaatatcaccatcgtggaatcaaacgagatcatcactggtatgccagcctcaactgaaggagcatatgtgtcttcagagtctcccattagaatatcagtatccacagaaggagcaaatacttcttcatctacatctacatccaccactgggacaagccatcttgtaaaatgtgcggagaaggagaaaactttctgtgtgaatggaggggagtgcttcatggtgaaagacctttcaaacccctcgagatacttgtgcaagtgcccaaatgagtttactggtgatcgctgccaaaactacgtaatggccagcttctacaagcatcttgggattgaatttatggaggcggaggagctgtaccagaagagagtgctgaccataaccggcatctgcatcgccctccttgtggtcggcatcatgtgtgtggtggcctactgcaaaaccaagaaacagcggaaaaagctgcatgaccgtcttcggcagagccttcggtctgaacgaaacaatatgatgaacattgccaatgggcctcaccatcctaacccaccccccgagaatgtccagctggtgaatcaatacgtatctaaaaacgtcatctccagtgagcatattgttgagagagaagcagagacatccttttccaccagtcactatacttccacagcccatcactccactactgtcacccagactcctagccacagctggagcaacggacacactgaaagcatcctttccgaaagccactctgtaatcgtgatgtcatccgtagaaaacagtaggcacagcagcccaactgggggcccaagaggacgtcttaatggcacaggaggccctcgtgaatgtaacagcttcctcaggcatgccagagaaacccctgattcctaccgagactctcctcatagtgaaaggtatgtgtcagccatgaccaccccggctcgtatgtcacctgtagatttccacacgccaagctcccccaaatcgcccccttcggaaatgtctccacccgtgtccagcatgacggtgtccatgccttccatggcggtcagccccttcatggaagaagagagacctctacttctcgtgacaccaccaaggctgcgggagaagaagtttgaccatcaccctcagcagttcagctccttccaccacaaccccgcgcatgacagtaacagcctccctgctagccccttgaggatagtggaggatgaggagtatgaaacgacccaagagtacgagccagcccaagagcctgttaagaaactcgccaatagccggcgggccaaaagaaccaagcccaatggccacattgctaacagattggaagtggacagcaacacaagctcccagagcagtaactcagagagtgaaacagaagatgaaagagtaggtgaagatacgcctttcctgggcatacagaaccccctggcagccagtcttgaggcaacacctgccttccgcctggctgacagcaggactaacccagcaggccgcttctcgacacaggaagaaatccaggccaggctgtctagtgtaattgctaaccaagaccctattgctgtataaaacctaaataaacacatagattcacctgtaaaactttattttatataataaagtattccaccttaaattaaacaatttattttattttagcagttctgcaaatagaaaacaggaaaaaaacttttataaattaaatatatgtatgtaaaaatgaaaaaaaaaaaaaaaa

SEQ ID NO: 5 - protein, 637 aa, Homo sapiens
MSERKEGRGKGKGKKKERGSGKKPESAAGSQSPALPPQLKEMKSQESAAGSKLVLRCETSSEYSSLRFKWFKNGNELNRKNKPQNIKIQKKPGKSELRINKASLADSGEYMCKVISKLGNDSASANITIVESNEIITGMPASTEGAYVSSESPIRISVSTEGANTSSSTSTSTTGTSHLVKCAEKEKTFCVNGGECFMVKDLSNPSRYLCKCPNEFTGDRCQNYVMASFYKAEELYQKRVLTITGICIALLVVGIMCVVAYCKTKKQRKKLHDRLRQSLRSERNNMMNIANGPHHPNPPPENVQLVNQYVSKNVISSEHIVEREAETSFSTSHYTSTAHHSTTVTQTPSHSWSNGHTESILSESHSVIVMSSVENSRHSSPTGGPRGRLNGTGGPRECNSFLRHARETPDSYRDSPHSERYVSAMTTPARMSPVDFHTPSSPKSPPSEMSPPVSSMTVSKPSMAVSPFMEEERPLLLVTPPRLREKKFDHHPQQFSSFHHNPAHDSNSLPASPLRIVEDEEYETTQEYEPAQEPVKKLANSRRAKRTKPNGHIANRLEVDSNTSSQSSNSESETEDERVGEDTPFLGIQNPLAASLEATPAFRLADSRTNPAGRFSTQEEIQARLSSVIANQDPIAV

SEQ ID NO: 6 - DNA, 2460 bp, Homo sapiens
  CDS (461)...(2371)
gtggctgcggggcaattgaaaaagagccggcgaggagttccccgaaacttgttggaactccgggctcgcgcggaggccaggagctgagcggcggcggctgccggacgatgggagcgtgagcaggacggtgataacctctccccgatcgggttgcgagggcgccgggcagaggccaggacgcgagccgccagcggcgggacccatcgacgacttcccggggcgacaggagcagccccgagagccagggcgagcgcccgttccaggtggccggaccgcccgccgcgtccgcgccgcgctccctgcaggcaacgggagacgcccccgcgcagcgcgagcgcctcagcgcggccgctcgctctccccatcgagggacaaacttttcccaaacccgatccgagcccttggaccaaactcgcctgcgccgagagccgtccgcgtagagcgctccgtctccggcgagatgtccgagcgcaaagaaggcagaggcaaagggaagggcaagaagaaggagcgaggctccggcaagaagccggagtccgcggcgggcagccagagcccagccttgcctccccaattgaaagagatgaaaagccaggaatcggctgcaggttccaaactagtccttcggtgtgaaaccagttctgaatactcctctctcagattcaagtggttcaagaatgggaatgaattgaatcgaaaaaacaaaccacaaaatatcaagatacaaaaaaagccagggaagtcagaacttcgcattaacaaagcatcactggctgattctggagagtatatgtgcaaagtgatcagcaaattaggaaatgacagtgcctctgccaatatcaccatcgtggaatcaaacgagatcatcactggtatgccagcctcaactgaaggagcatatgtgtcttcagagtctcccattagaatatcagtatccacagaaggagcaaatacttcttcatctacatctacatccaccactgggacaagccatcttgtaaaatgtgcggagaaggagaaaactttctgtgtgaatggaggggagtgcttcatggtgaaagacctttcaaacccctcgagatacttgtgcaagtgcccaaatgagtttactggtgatcgctgccaaaactacgtaatggccagcttctacaaggcggaggagctgtaccagaagagagtgctgaccataaccggcatctgcatcgccctccttgtggtcggcatcatgtgtgtggtggcctactgcaaaaccaagaaacagcggaaaaagctgcatgaccgtcttcggcagagccttcggtctgaacgaaacaatatgatgaacattgccaatgggcctcaccatcctaacccaccccccgagaatgtccagctggtgaatcaatacgtatctaaaaacgtcatctccagtgagcatattgttgagagagaagcagagacatccttttccaccagtcactatacttccacagcccatcactccactactgtcacccagactcctagccacagctggagcaacggacacactgaaagcatcctttccgaaagccactctgtaatcgtgatgtcatccgtagaaaacagtaggcacagcagcccaactgggggcccaagaggacgtcttaatggcacaggaggccctcgtgaatgtaacagcttcctcaggcatgccagagaaacccctgattcctaccgagactctcctcatagtgaaaggtatgtgtcagccatgaccaccccggctcgtatgtcacctgtagatttccacacgccaagctcccccaaatcgcccccttcggaaatgtctccacccgtgtccagcatgacggtgtccaagccttccatggcggtcagccccttcatggaagaagagagacctctacttctcgtgacaccaccaaggctgcgggagaagaagtttgaccatcaccctcagcagttcagctccttccaccacaaccccgcgcatgacagtaacagcctccctgctagccccttgaggatagtggaggatgaggagtatgaaacgacccaagagtacgagccagcccaagagcctgttaagaaactcgccaatagccggcgggccaaaagaaccaagcccaatggccacattgctaacagattggaagtggacagcaacacaagctcccagagcagtaactcagagagtgaaacagaagatgaaagagtaggtgaagatacgcctttcctgggcatacagaaccccctggcagccagtcttgaggcaacacctgccttccgcctggctgacagcaggactaacccagcaggccgcttctcgacacaggaagaaatccaggccaggctgtctagtgtaattgctaaccaagaccctattgctgtataaaacctaaataaacacatagattcacctgtaaaactttattttatataataaagtattccaccttaaattaaacaatttattttatt

SEQ ID NO: 7 - protein, 241 aa, Homo sapiens
MSERKEGRGKGKGKKKERGSGKKPESAAGSQSPALPPQLKEMKSQESAAGSKLVLRCETSSEYSSLRFKWFKNGNELNRKNKPQNIKIQKKPGKSELRINKASLADSGEYMCKVISKLGNDSASANITIVESNEIITGMPASTEGAYVSSESPIRISVSTEGANTSSSTSTSTTGTSHLVKCAEKEKTFCVNGGECFMVKDLSNPSRYLCKCPNEFTGDRCQNYVMASFYSTSTPFLSLPE

SEQ ID NO: 8 - DNA, 1715 bp, Homo sapiens
  CDS (493)...(1215)
gcgcctgcctccaacctgcgggcgggaggtgggtggctgcggggcaattgaaaaagagccggcgaggagttccccgaaacttgttggaactccgggctcgcgcggaggccaggagctgagcggcggcggctgccggacgatgggagcgtgagcaggacggtgataacctctccccgatcgggttgcgagggcgccgggcagaggccaggacgcgagccgccagcggcgggacccatcgacgacttcccggggcgacaggagcagccccgagagccagggcgagcgcccgttccaggtggccggaccgcccgccgcgtccgcgccgcgctccctgcaggcaacgggagacgcccccgcgcagcgcgagcgcctcagcgcggccgctcgctctccccatcgagggacaaacttttcccaaacccgatccgagcccttggaccaaactcgcctgcgccgagagccgtccgcgtagagcgctccgtctccggcgagatgtccgagcgcaaagaaggcagaggcaaagggaagggcaagaagaaggagcgaggctccggcaagaagccggagtccgcggcgggcagccagagcccagccttgcctccccaattgaaagagatgaaaagccaggaatcggctgcaggttccaaactagtccttcggtgtgaaaccagttctgaatactcctctctcagattcaagtggttcaagaatgggaatgaattgaatcgaaaaaacaaaccacaaaatatcaagatacaaaaaaagccagggaagtcagaacttcgcattaacaaagcatcactggctgattctggagagtatatgtgcaaagtgatcagcaaattaggaaatgacagtgcctctgccaatatcaccatcgtggaatcaaacgagatcatcactggtatgccagcctcaactgaaggagcatatgtgtcttcagagtctcccattagaatatcagtatccacagaaggagcaaatacttcttcatctacatctacatccaccactgggacaagccatcttgtaaaatgtgcggagaaggagaaaactttctgtgtgaatggaggggagtgcttcatggtgaaagacctttcaaacccctcgagatacttgtgcaagtgcccaaatgagtttactggtgatcgctgccaaaactacgtaatggccagcttctacagtacgtccactccctttctgtctctgcctgaataggagcatgctcagttggtgctgctttcttgttgctgcatctcccctcagattccacctagagctagatgtgtcttaccagatctaatattgactgcctctgcctgtcgcatgagaacattaacaaaagcaattgtattacttcctctgttcgcgactagttggctctgagatactaataggtgtgtgaggctccggatgtttctggaattgatattgaatgatgtgatacaaattgatagtcaatatcaagcagtgaaatatgataataaaggcatttcaaagtctcacttttattgataaaataaaaatcattctactgaacagtccatcttctttatacaatgaccacatcctgaaaagggtgttgctaagctgtaaccgatatgcacttgaaatgatggtaagttaattttgattcagaatgtgttatttgtcacaaataaacataataaaaggagttcagatgtttttcttcattaaccaaaaaaaaaaaaaaa

SEQ ID NO: 9 - protein, 420 aa, Homo sapiens
MSERKEGRGKGKGKKKERGSGKKPESAAGSQSPALPPQLKEMKSQESAAGSKLVLRCETSSEYSSLRFKWFKNGNELNRKNKPQNIKIQKKPGKSELRINKASLADSGEYMCKVISKLGNDSASANITIVESNEIITGMPASTEGAYVSSESPIRISVSTEGANTSSSTSTSTTGTSHLVKCAEKEKTFCVNGGECFMVKDLSNPSRYLCKCPNEFTGDRCQNYVMASFYKAEELYQKRVLTITGICIALLVVGIMCVVAYCKTKKQRKKLHDRLRQSLRSERNNMMNIANGPHHPNPPPENVQLVNQYVSKNVISSEHIVEREAETSFSTSHYTSTAHHSTTVTQTPSHSWSNGHTESILSESHSVIVMSSVENSRHSSPTGGPRGRLNGTGGPRECNSFLRHARETPDSYRDSPHSER

SEQ ID NO: 10 - DNA, 2431 bp, Homo sapiens
  CDS (496)...(1755)
gaggcgcctgcctccaacctgcgggcgggaggtgggtggctgcggggcaattgaaaaagagccggcgaggagttccccgaaacttgttggaactccgggctcgcgcggaggccaggagctgagcggcggcggctgccggacgatgggagcgtgagcaggacggtgataacctctccccgatcgggttgcgagggcgccgggcagaggccaggacgcgagccgccagcggcgggacccatcgacgacttcccggggcgacaggagcagccccgagagccagggcgagcgcccgttccaggtggccggaccgcccgccgcgtccgcgccgcgctccctgcaggcaacgggagacgcccccgcgcagcgcgagcgcctcagcgcggccgctcgctctccccatcgagggacaaacttttcccaaacccgatccgagcccttggaccaaactcgcctgcgccgagagccgtccgcgtagagcgctccgtctccggcgagatgtccgagcgcaaagaaggcagaggcaaagggaagggcaagaagaaggagcgaggctccggcaagaagccggagtccgcggcgggcagccagagcccagccttgcctccccaattgaaagagatgaaaagccaggaatcggctgcaggttccaaactagtccttcggtgtgaaaccagttctgaatactcctctctcagattcaagtggttcaagaatgggaatgaattgaatcgaaaaaacaaaccacaaaatatcaagatacaaaaaaagccagggaagtcagaacttcgcattaacaaagcatcactggctgattctggagagtatatgtgcaaagtgatcagcaaattaggaaatgacagtgcctctgccaatatcaccatcgtggaatcaaacgagatcatcactggtatgccagcctcaactgaaggagcatatgtgtcttcagagtctcccattagaatatcagtatccacagaaggagcaaatacttcttcatctacatctacatccaccactgggacaagccatcttgtaaaatgtgcggagaaggagaaaactttctgtgtgaatggaggggagtgcttcatggtgaaagacctttcaaacccctcgagatacttgtgcaagtgtccaaatgagtttactggtgatcgctgccaaaactacgtaatggccagcttctacaaggcggaggagctgtaccagaagagagtgctgaccataaccggcatctgcatcgccctccttgtggtcggcatcatgtgtgtggtggcctactgcaaaaccaagaaacagcggaaaaagctgcatgaccgtcttcggcagagccttcggtctgaacgaaacaatatgatgaacattgccaatgggcctcaccatcctaacccaccccccgagaatgtccagctggtgaatcaatacgtatctaaaaacgtcatctccagtgagcatattgttgagagagaagcagagacatccttttccaccagtcactatacttccacagcccatcactccactactgtcacccagactcctagccacagctggagcaacggacacactgaaagcatcctttccgaaagccactctgtaatcgtgatgtcatccgtagaaaacagtaggcacagcagcccaactgggggcccaagaggacgtcttaatggcacaggaggccctcgtgaatgtaacagcttcctcaggcatgccagagaaacccctgattcctaccgagactctcctcatagtgaaaggtaaaaccgaaggcaaagctactgcagaggagaaactcagtcagagaatccctgtgagcacctgcggtctcacctcaggaaatctactctaatcagaataaggggcggcagttacctgttctaggagtgctcctagttgatgaagtcatctctttgtttgacggaacttatttcttctgagcttctctcgtcgtcccagtgactgacaggcaacagactcttaaagagctgggatgctttgatgcggaaggtgcagcacatggagtttccagctctggccatgggctcagacccactcggggtctcagtgtcctcagttgtaacattagagagatggcatcaatgcttgataaggacccttctataattccaattgccagttatccaaactctgattcggtggtcgagctggcctcgtgttcttatctgctaaccctgtcttaccttccagcctcagttaagtcaaatcaagggctatgtcattgctgaatgtcatggggggcaactgcttgccctccaccctatagtatctattttatgaaattccaagaagggatgaataaataaatctcttggatgctgcgtctggcagtcttcacgggtggttttcaaagcagaaaaaaaaaaaaaaaaaaaaaaaaaaaaaaaaaaaaaaaaaaaaaaaaaaaaaaaaaaaa

SEQ ID NO: 11 - protein, 768 aa, Homo sapiens
MDVKERKPYRSLTRRRDAERRYTSSSADSEEGKAPQKSYSSSETLKAYDQDARLAYGSRVKDIVPQEAEEFCRTGANFTLRELGLEEVTPPHGTLYRTDIGLPHCGYSMGAGSDADMEADTVLSPEHPVRLWGRSTRSGRSSCLSSRANSNLTLTDTEHENTETDHPGGLQNHARLRTPPPPLSHAHTPNQHHAASINSLNRGNFTPRSNPSPAPTDHSLSGEPPAGGAQEPAHAQENWLLNSNIPLETRNLGKQPFLGTLQDNLIEMDILGASRHDGAYSDGHFLFKPGGTSPLFCTTSPGYPLTSSTVYSPPPRPLPRSTFARPAFNLKKPSKYCNWKCAALSAIVISATLVILLAYFVAMHLFGLNWHLQPMEGQMYEITEDTASSWPVPTDVSLYPSGGTGLETPDRKGKGTTEGKPSSFFPEDSFIDSGEIDVGRRASQKIPPGTFWRSQVFIDHPVHLKFNVSLGKAALVGIYGRKGLPPSHTQFDFVELLDGRRLLTQEARSLEGTPRQSRGTVPPSSHETGFIQYLDSGIWHLAFYNDGKESEVVSFLTTAIALPPRLKEMKSQESAAGSKLVLRCETSSEYSSLRFKWFKNGNELNRKNKPQNIKIQKKPGKSELRINKASLADSGEYMCKVISKLGNDSASANITIVESNEIITGMPASTEGAYVSSESPIRISVSTEGANTSSSTSTSTTGTSHLVKCAEKEKTFCVNGGECFMVKDLSNPSRYLCKCPNEFTGDRCQNYVMASFYSTSTPFLSLPE

SEQ ID NO: 12 - DNA, 3111 bp, Homo sapiens
  CDS (334)...(2637)
gggtaccatgggtcggtgagcgcgtttcccgcctgagcgcaactagcggcgggtcgtgggcacctccagaaaagatcccgcaccatcctccaggatccaatggccttggagagagggctgcagggcccacggacattgctgactcttcagaacgtgctgacatggagccaggtagactgaaattatcatgtgtccaaattaaaattgcatacttcaaggattatttgaaggactattcttagacccttttaagaagatttaaagaaaaaccactcggccctgagtgcggcgaggaccctgtttgtggatgtggaggagcgcgggccggaggccatggacgtgaaggagaggaagccttaccgctcgctgacccggcgccgcgacgccgagcgccgctacaccagctcgtccgcggacagcgaggagggcaaagccccgcagaaatcgtacagctccagcgagaccctgaaggcctacgaccaggacgcccgcctagcctatggcagccgcgtcaaggacattgtgccgcaggaggccgaggaattctgccgcacaggtgccaacttcaccctgcgggagctggggctggaagaagtaacgccccctcacgggaccctgtaccggacagacattggcctcccccactgcggctactccatgggggctggctctgatgccgacatggaggctgacacggtgctgtcccctgagcaccccgtgcgtctgtggggccggagcacacggtcagggcgcagctcctgcctgtccagccgggccaattccaatctcacactcaccgacaccgagcatgaaaacactgagactgatcatccgggcggcctgcagaaccacgcgcggctccggacgccgccgccgccgctctcgcacgcccacacccccaaccagcaccacgcggcctccattaactccctgaaccggggcaacttcacgccgaggagcaaccccagcccggcccccacggaccactcgctctccggagagccccctgccggcggcgcccaggagcctgcccacgcccaggagaactggctgctcaacagcaacatccccctggagaccagaaacctaggcaagcagccattcctagggacattgcaggacaacctcattgagatggacattctcggcgcctcccgccatgatggggcttacagtgacgggcacttcctcttcaagcctggaggcacctccccgctcttctgcaccacatcaccagggtacccactgacgtccagcacagtgtactctcctccgccccgacccctgccccgcagcaccttcgcccggccggcctttaacctcaagaagccctccaagtactgtaactggaagtgcgcagccctgagcgccatcgtcatctcagccactctggtcatcctgctggcatactttgtggccatgcacctgtttggcctaaactggcacctgcagccgatggaggggcagatgtatgagatcacggaggacacagccagcagttggcctgtgccaaccgacgtctccctatacccctcagggggcactggcttagagacccctgacaggaaaggcaaaggaaccacagaaggaaagcccagtagtttctttccagaggacagtttcatagattctggagaaattgatgtgggaaggcgagcttcccagaagattcctcctggcactttctggagatctcaagtgttcatagaccatcctgtgcatctgaaattcaatgtgtctctgggaaaggcagccctggttggcatttatggcagaaaaggcctccctccttcacatacacagtttgactttgtggagctgctggatggcaggaggctcctaacccaggaggcgcggagcctagaggggaccccgcgccagtctcggggaactgtgcccccctccagccatgagacaggcttcatccagtatttggattcaggaatctggcacttggctttttacaatgacggaaaggagtcagaagtggtttcctttctcaccactgccattgccttgcctccccgattgaaagagatgaaaagccaggaatcggctgcaggttccaaactagtccttcggtgtgaaaccagttctgaatactcctctctcagattcaagtggttcaagaatgggaatgaattgaatcgaaaaaacaaaccacaaaatatcaagatacaaaaaaagccagggaagtcagaacttcgcattaacaaagcatcactggctgattctggagagtatatgtgcaaagtgatcagcaaattaggaaatgacagtgcctctgccaatatcaccatcgtggaatcaaacgagatcatcactggtatgccagcctcaactgaaggagcatatgtgtcttcagagtctcccattagaatatcagtatccacagaaggagcaaatacttcttcatctacatctacatccaccactgggacaagccatcttgtaaaatgtgcggagaaggagaaaactttctgtgtgaatggaggggagtgcttcatggtgaaagacctttcaaacccctcgagatacttgtgcaagtgcccaaatgagtttactggtgatcgctgccaaaactacgtaatggccagcttctacagtacgtccactccctttctgtctctgcctgaataggagcatgctcagttggtgctgctttcttgttgctgcatctcccctcagattccacctagagctagatgtgtcttaccagatctaatattgactgcctctgcctgtcgcatgagaacattaacaaaagcaattgtattacttcctctgttcgcgactagttggctctgagatactaataggtgtgtgaggctccggatgtttctggaattgatattgaatgatgtgatacaaattgatagtcaatatcaagcagtgaaatatgataataaaggcatttcaaagtctcacttttattgataaaataaaaatcattctactgaacagtccatcttctttatacaatgaccacatcctgaaaagggtgttgctaagctgtaaccgatatgcacttgaaatgatggtaagttaattttgattcagaatgtgttatttgtcacaaataaacataataaaaggaaaaaaaaaaaaaaa

SEQ ID NO: 13 - DNA, 1872 bp, Homo sapiens
  CDS (507)...(1394)
gaattcgggacagcctctcctgccgccgctgctgctgccgccgccgccaccgccggctggtcctccttctgcttttacttctcctgcatgacagttgttttcttcatctgagcagacaccagcttcagatgctcgaggtgagaaacatgcctttcagtttgggctactggtttacttaattaatcagccggcagctccgtcgatctattttcgtccctgtcctcttgacgagcccgggatggtttggagtagcatttaaaagaactagaaaagtggcccagaaacagcagcttaaagaattattacgatatactttgattttgtagttgctaggagcttttcttccccccttgcatctttctgaactcttcttgattttaataatggccttggacttggacgatttatcgatttccccctgtaagatgctgtatcatttggttgggggggcctctgcgtggtaatggaccgtgagagcggccaggccttcttctggaggtgagccgatggagatttattccccagacatgtctgaggtcgccgccgagaggtcctccagcccctccactcagctgagtgcagacccatctcttgatgggcttccggcagcagaagacatgccagagccccagactgaagatgggagaacccctggactcgtgggcctggccgtgccctgctgtgcgtgcctagaagctgagcgcctgagaggttgcctcaactcagagaaaatctgcattgtccccatcctggcttgcctggtcagcctctgcctctgcatcgccggcctcaagtgggtatttgtggacaagatctttgaatatgactctcctactcaccttgaccctggggggttaggccaggaccctattatttctctggacgcaactgctgcctcagctgtgtgggtgtcgtctgaggcatacacttcacctgtctctagggctcaatctgaaagtgaggttcaagttacagtgcaaggtgacaaggctgttgtctcctttgaaccatcagcggcaccgacaccgaagaatcgtatttttgccttttctttcttgccgtccactgcgccatccttcccttcacccacccggaaccctgaggtgagaacgcccaagtcagcaactcagccacaaacaacagaaactaatctccaaactgctcctaaactttctacatctacatccaccactgggacaagccatcttgtaaaatgtgcggagaaggagaaaactttctgtgtgaatggaggggagtgcttcatggtgaaagacctttcaaacccctcgagatacttgtgcaagtgcccaaatgagtttactggtgatcgctgccaaaactacgtaatggccagcttctacagtacgtccactccctttctgtctctgcctgaataggagcatgctcagttggtgctgctttcttgttgctgcatctcccctcagattccacctagagctagatgtgtcttaccagatctaatattgactgcctctgcctgtcgcatgagaacattaacaaaagcaattgtattacttcctctgttcgcgactagttggctctgagatactaataggtgtgtgaggctccggatgtttctggaattgatattgaatgatgtgatacaaattgatagtcaatatcaagcagtgaaatatgataataaaggcatttcaaagtctcacttttattgataaaataaaaatcattctactgaacagtccatcttctttatacaatgaccacatcctgaaaagggtgttgctaagctgtaaccgatatgcacttgaaatgatggtaagttaattttgattcagaatgtgttatttgtcacaaataaacataataaaaggaaaaaaaaaacccgaattc

SEQ ID NO: 14 - protein, 296 aa, Homo sapiens
MEIYSPDMSEVAAERSSSPSTQLSADPSLDGLPAAEDMPEPQTEDGRTPGLVGLAVPCCACLEAERLRGCLNSEKICIVPILACLVSLCLCIAGLKWVFVDKIFEYDSPTHLDPGGLGQDPIISLDATAASAVWVSSEAYTSPVSRAQSESEVQVTVQGDKAVVSFEPSAAPTPKNRIFAFSFLPSTAPSFPSPTRNPEVRTPKSATQPQTTETNLQTAPKLSTSTSTTGTSHLVKCAEKEKTFCVNGGECFMVKDLSNPSRYLCKCPNEFTGDRCQNYVMASFYSTSTPFLSLPE